(19) United States
(12) Patent Application Publication
(10) Pub. No.: US 2006/0029617 A1
(43) Pub. Date: Feb. 9, 2006

(54) Porcine circovirus and Helicobacter combination vaccines and methods of use

(76) Inventors: Catherine Elisabeth Charreyre, Saint-Laurent de Mure (FR); John Ellis, Saskatoon (CA); Olivier Michel Martinon, Athens, GA (US)

Correspondence Address: Judy Jarecki-Black, Ph.D,J.D, 3239 Satellite Blvd., Duluth, GA, 30096, US

(21) Appl. No.: 11/107,219

(22) Filed: Apr. 15, 2005

Related U.S. Application Data
(63) Continuation-in-part of application No. 10/653,849, filed on Sep. 2, 2003.
Application No. 10/653,849 is a continuation-in-part of application No. 10/334,245, filed on Dec. 31, 2002.
Application No. 10/334,245 is a continuation of application No. 09/935,428, filed on Aug. 20, 2001, now abandoned.
Application No. 09/935,428 is a continuation of application No. 09/209,961, filed on Dec. 10, 1998, now abandoned.
(63) Continuation-in-part of application No. 09/884,514, filed on Jun. 19, 2001, now Pat. No. 6,660,272.
Application No. 09/884,514 is a division of application No. 09/161,092, filed on Sep. 25, 1998, now Pat. No. 6,391,314.
Application No. 09/161,092 is a continuation-in-part of application No. 09/082,558, filed on May 21, 1998, now Pat. No. 6,368,601.
(63) Continuation-in-part of application No. 09/680,228, filed on Oct. 6, 2000, now abandoned.
Application No. 09/680,228 is a continuation-in-part of application No. 09/583,350, filed on May 31, 2000, now Pat. No. 6,517,843.
(63) Continuation-in-part of application No. 09/784,962, filed on Feb. 16, 2001, now Pat. No. 6,953,581.
Application No. 09/784,962 is a division of application No. 09/347,594, filed on Jul. 1, 1999, now Pat. No. 6,217,883.
(60) Provisional application No. 60/069,750, filed on Dec. 16, 1997.
(60) Provisional application No. 60/069,233, filed on Dec. 11, 1997.
(60) Provisional application No. 60/151,564, filed on Aug. 31, 1999.

(30) Foreign Application Priority Data
Oct. 3, 1997 (FR) 97/12382
Jan. 22, 1998 (FR) 98/00873
Mar. 20, 1998 (FR) 98/03707
Jul. 6, 1998 (FR) 98/08777
Dec. 11, 1998 (WO) PCT/CA98/01130

(51) Int. Cl. C12Q 1/70; A61K 39/295

(52) U.S. Cl. 424/201.1; 435/5

(57) ABSTRACT

The present invention is based on the discovery of novel species of the genus Helicobacter that are associated with gastro-esophageal ulceration in pigs. In particular, a novel species, H. cerdo, has been used as a source of antigenic material for the development of vaccine for the treatment of the gastro-esophageal disorders. Most advantageously, the novel Helicobacter and the porcine circoviruses associated with PMWS in pigs are useful for providing combination vaccines whereby immunogens derived from both types of pathogens may be codelivered to the target animal to stimulate the generation of protective antibodies and immunity. The invention, therefore, provides vaccines that are useful for the tratment of gastro-esophageal ulceration and PMWS in porcines. The present invention includes, therefore, multivalent immunogenic compositions and vaccines, multivaccine kits, and combined immunization or vaccination methods which make it possible to use such combined immunization or vaccination programmes.

### CROSS-REFERENCE TO RELATED APPLICATIONS

This application is a continuation-in-part of pending U.S. application Ser. No. 10/653,849, filed Sep. 2, 2003, which is a continuation-in-part of pending U.S. application Ser. No. 10/334,245, filed on Dec. 31, 2002, which is a continuation of abandoned U.S. application Ser. No. 09/935,428, filed Aug. 20, 2001, which is a continuation of abandoned U.S. application Ser. No. 09/209,961, filed Dec. 10, 1998, which claims priority to U.S. application Ser. No. 60/069,750, filed Dec. 16, 1997, and to U.S. application Ser. No. 60/069,233, filed Dec. 11, 1997.

This application is also a continuation-in-part of pending U.S. application Ser. No. 09/884,514, filed Jun. 19, 2001, which is a divisional of U.S. application Ser. No. 09/161,092, filed Sep. 25, 1998, now U.S. Pat. No. 6,391,314, which is a continuation-in-part of U.S. application Ser. No. 09/082,558, filed May 21, 1998, now U.S. Pat. No. 6,368,601, which claims priority to French Applications 97/12382, filed Oct. 3, 1997; 98/00873, filed Jan. 22, 1998 and 98/03707, filed Mar. 20, 1998.

This application is also a continuation-in-part of pending U.S. application Ser. No. 09/680,228, filed Oct. 6, 2000, which is a continuation-in-part of U.S. application Ser. No. 09/583,350, filed May 31, 2000, now U.S. Pat. No. 6,517,843, which claims priority to U.S. application Ser. No. 60/151,564, filed Aug. 31, 1999. This application is also a continuation-in-part of pending U.S. application Ser. No. 09/784,962, filed Feb. 16, 2001, which is a divisional of U.S. application Ser. No. 09/347,594, filed Jul. 1, 1999, now U.S. Pat. No. 6,217,883, which claims priority to French Application 98/08777, filed Jul. 6, 1998. This application is related to International application Serial No. PCT/CA98/01130, filed Dec. 11, 1998.

### INCORPORATION BY REFERENCE

All documents cited therein or during their prosecution (“application cited documents”) and all documents cited or referenced in the application cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention

### FIELD OF THE INVENTION

The present invention relates generally to combination vaccines. More particularly, the present invention pertains to the isolation and characterization of new porcine Helicobacter species isolated from pigs with gastro-esophageal ulceration, vaccines thereof and combination vaccines directed against a Helicobacter species and a porcine circovirus strain.

### BACKGROUND OF THE INVENTION

Postweaning multisystemic wasting syndrome (PMWS) is a newly emerged disease of pigs. PMWS appears to destroy the host immune system and causes a high mortality rate in weaned pigs. This disease has a long incubation period, typically 3-8 weeks, and affects many organs of infected pigs. The PMWS syndrome detected in Canada, the United States and France is clinically characterized by a gradual loss of weight and by manifestations such as tachypnea, dyspnea and jaundice. From the pathological point of view, it is manifested by lymphocytic or granulomateus infiltrations, lymphadenopathies and, more rarely, by hepatitis and lymphocytic or granulomateus nephritis (Clark, Proc. Am. Assoc. Swine Prac. 1997; 499-501; La Semaine Veterinaire No. 26, supplement to La Semaine Veterinaire 1996 (834); La Semaine Veterinaire 1997 (857): 54; Gupi P. S. Nayar et al., Can. Vet. J, vol. 38, 1997; 385-387). PMWS-affected piglets often die from respiratory failure and interstitial pneumonia with histiocytic cell infiltration.

Porcine circovirus (PCV) causes worldwide infection in swine and is highly contagious. PCV was originally detected as a noncytopathic contaminant of porcine kidney (PK15) cell lines. PCV has been classified into the new virus family Circoviridae. These viruses are small, nonenveloped agents with a single-stranded circular DNA genome.

A variety of circoviruses have been identified in a range of animal species including PCV, chicken anemia virus (CAV), beak and feather disease virus (BFDV) of psittacine birds, plant viruses including subterranean clover stunt virus (SCSV), coconut foliar decay virus (CFDV) and banana bunch top virus (BBTV). There do not appear to be DNA sequence homologies or common antigenic determinants among the currently recognized circoviruses. Todd et al. (1991) Arch. Virol. 117:129-135.

Members in the circovirus family have been shown to cause anemia, immunodeficiency-related diseases and to infect macrophage cells in vitro. PCV has only recently been implicated in PMWS. See, e.g., Ellis et al. (1998) Can. Vet. J. 39:44-51 and Gopi et al. (1997) Can. Vet. J. 38:385-386. However, the etiologic association of PCV with PMWS has been questioned due to the ubiquitous presence of PCV in the pig population. Additionally, experimental infections of pigs with PCV inocula, derived from contaminated PK15 cell cultures, have failed to produce clinical disease. See, e.g., Tischer et al. (1986) Arch. Virol. 91:271-276.

Infectious agents of swine, especially viruses, not only profoundly affect the farming industry, but pose potential public health risks to humans, due to the increased interest in the use of pig organs for xenotransplantation in humans. Previous diagnosis of PMWS disease has been based on histopathological examination. Accordingly, there is a need for improved methods of diagnosing the presence of PMWS-associated pathogens, as well as for preventing PMWS disease.

In the U.S.A. fifty-nine million hogs are processed annually and direct death loss from gastro-esophageal ulceration is estimated as at least 2.0% and may be as high as 5%. The financial loss is at least $147 million annually. Subclinical losses due to unthriftiness and to carcass condemnations secondary to tail biting are unknown but could be huge, as much as $750 million per year. Gastro-esophageal ulcerative disease in pigs typically occurs in 5-90% of 3-6 month old swine with annual death losses of 1-5%. The usual symptoms are sudden death or anemia, anorexia, vomiting and unthriftiness with melena, tail biting and carcass condemnations. Factors known to influence the prevalence of GEU include a high carbohydrate diets, feed particle size, social stress, and gastric microbes. “Many of the techniques used to increase feed efficiency and reduce feeding costs are associated with an increased prevalence of stomach lesions. Economics dictate that a compromise between feed eficeincy and losses due to ulcers must be reached.” R. Friendship in “Diseases of Swine”, 8^{th} ed., ISU Press, Ames, Iowa.

In humans, bacterial gastritis associated with Helicobacter pylori infection is now recognized as the cause of type B gastritis associated with gastric carcinoma and MALT lymphoma. This is the most common bacterial infectious disease, although decreasing in frequency in the developed world. Antimicrobial drugs are effective treatments but inbalance of the “normal” gastric bacterial populations may lead to gastroduodenal ulceration and gastric reflux disease linked to adenocarcinomas. The anatomic and physiological similarities between humans and pigs suggest that there might be a bacterial origin for gastro-esophageal ulceration in porcines. However, perturbations in the microbial flora due to antimicrobial drugs would also be undesireable. There exists a need, therefore, for vaccines directed against porcine gastric flora, and in particular those bacteria such as Helicobacter species that are causatively associated with gastricesophageal disorders.

Citation or identification of any document in this application is not an admission that such document is available as prior art to the present invention.

### SUMMARY OF THE INVENTION

The present invention is based on the discovery of a new virus, designated “PCV Type II” or “PCVII” herein, isolated from homogenized tissues of PMWS-affected piglets. Characterization of the virus shows that it shares common features with the nonpathogenic porcine circovirus obtained from persistently infected PK15 cells, designated “PCV Type I” or “PCVI” herein. The entire DNA genome of a novel PCV variant, PCVII 412, as well as several additional PCVII isolates, have been cloned and sequenced. Portions of these DNA sequences are useful as probes to diagnose the presence of virus in clinical samples, and to isolate other naturally occurring variants of the virus. An understanding of the genomic sequence of PCVII also makes available the polypeptide sequences of the various proteins encoded within the open reading frames of the viral genome and permits production of these peptides or portions thereof which are useful as standards or reagents in diagnostic tests and as components of vaccines. Protective antibodies may also be raised from the proteins and may be produced in polyclonal or monoclonal form.

The availability of the entire PCVII sequence thus permits the design and construction of polypeptides that may either serve as vaccines or diagnostic reagents, or as intermediates in the production of monoclonal antibody (Mab) preparations useful in passive immunotherapy against PMWS, or as intermediates in the production of antibodies useful as diagnostic reagents.

The present invention is further based on the discovery of novel bacterial species, in particular novel species of the genus Helicobacter that are associated with gastro-esophageal ulceration in pigs. In particular, a novel species, H. cerdo, has been used as a source of antigens for the development of vaccines for the treatment of the gastro-esophageal disorders. The present invention provides methods for deriving immunogenic compositions from H. cerdo and related species thereof by attenuation or chemical inactivation.

Most advantageously, the novel Helicobacter and porcine circoviruses are useful for providing combination vaccines whereby immunogens derived from both types of pathogens may be codelivered to the target animal to stimulate the generation of antibodies and immunity. The invention, therefore, provides vaccines that are useful for the tratment of gastro-esophageal ulceration and PMWS in porcines.

Accordingly, in one aspect, the invention relates to polynucleotides useful for the production of PCVII diagnostics and vaccines derived from the PCVII genome. In one particular embodiment, the polynucleotides are capable of selectively hybridizing to a PCVII nucleotide sequence and comprise at least about 8 contiguous nucleotides derived from, or complementary to, the sequences of the PCVII strains 412, 9741 and B9, 1010, 1011-48121, 1011-48285, 999, 1103 and 1121 (SEQ ID NOS: 1, 11, 12, and 24-30). In another embodiment, the polynucleotide encodes an immunogenic PCVII polypeptide having at least about 85% identity to a polypeptide selected from the group consisting of a polypeptide derived from open reading frame ORFs 1-6, and immunogenic fragments of ORFs 1-6 comprising at least about 5 amino acids. In an advantageous embodiment, the polynucleotide encodes the polypeptide of ORF 6, or immunogenic fragments thereof. The invention further relates to utilizing these polynucleotide sequences or portions thereof as oligomeric probes, for production of peptides which can serve as diagnostic reagents or as vaccine antigens, to the peptides themselves, and to polyclonal and monoclonal antibodies useful in diagnosis and treatment of the disease.

Other aspects of the invention include expression systems which are capable of effecting the production of a desired protein encoded by sequences derived from the complete PCVII genome, to recombinant vectors containing such systems or portions thereof, to recombinant host cells transformed with such vectors, to proteins produced by the transformed cells, and to vaccines prepared from such proteins. In addition, the invention relates to peptide sequences representing epitopes encoded by the genome, and to such sequences covalently linked to label or to carrier proteins. Also encompassed by the present invention are the various ORFs of the PCVII genome, as well as the proteins encoded by these ORFs, and portions thereof.

The invention also relates to the methods of preparing polypeptide compositions, such as vaccines and immunodiagnostic compositions, and immunoglobulins, and to immunoassays and kits for assays containing the primers, probes, polypeptides, and/or immunoglobulins. In one embodiment, then, the invention pertains to a method of detecting PCVII antibodies in a biological sample comprising (a) providing a biological sample; (b) reacting the biological sample with an immunogenic PCVII polypeptide as described above, under conditions which allow PCVII antibodies, when present in the biological sample, to bind to the PCVII polypeptide to form an antibody/antigen complex; and (c) detecting the presence or absence of the complex, thereby detecting the presence or absence of PCVII antibodies in the sample.

In the context of the combined immunization or vaccination programmes of the invention, it is also possible to combine the immunization or vaccination against the porcine circovirus with that against porcine Helicobacter and may further include an immunization or vaccination against other pig pathogens, in particular those which could be associated with the PMWS syndrome. An immunogenic composition or vaccine according to the invention may therefore comprise another valency corresponding to another pig pathogen such as, but not limited to, PRRS (Porcine Reproductory and Respiratory Syndrome) and/or Mycoplasma hyopneumoniae, and/or E. coli, and/or Atrophic Rhinitis, and/or Pseudorabies (Aujeszky's disease) virus and/or porcine influenza and/or Actinobacillus pleuropneumoniae and/or Hog cholera, and combinations thereof. Preferably, the programme of immunization or vaccination and the vaccines according to the invention will combine immunizations or vaccinations against the circovirus and the Helicobacter-generated gasro-esophageal ulceration, and the PRRS and/or porcine influenza. It is thus possible to use any appropriate form of immunogenic composition or vaccine, in particular any available commercial vaccine, so as to combine it with the immunogenic composition or vaccine against the porcine circovirus and porcine Helicobacter, as described herein.

The subject of the present invention encompasses therefore also multivalent immunogenic compositions and vaccines, multivaccine kits, and combined immunization or vaccination methods which make it possible to use such combined immunization or vaccination programmes.

One aspect of the invention, therefore, encompasses an immunogenic composition for eliciting an immunological response against a Helicobacter species and porcine circovirus comprising at least one Helicobacter antigen and at least one porcine circovirus antigen, and a veterinarily acceptable vehicle or excipient.

In one embodiment of the invention, the immunogenic composition, the porcine circovirus antigen may comprise at least one porcine circovirus type II antigen. In another embodiment of the invention, the Helicobacter antigen may be, but is not limited to, an antigen of Helicobacter cerdo, Helicobacter heilmanii, or Helicobacter pylori.

In the various embodiments of the invention, the porcine circovirus type II antigen encompasses at least one antigen of a porcine circovirus type II deposited at the ECACC selected from group consisting of: porcine circovirus type II accession No. V97100219, porcine circovirus type II accession No. V97100218, porcine circovirus type II accession No. V97100217, porcine circovirus type II accession No. V98011608, and porcine circovirus type II accession No. V98011609. In one embodiment of the invention, the porcine circovirus type II antigen is an attenuated virus porcine circovirus type II or an inactivated porcine circovirus type II or can comprise an antigen encoded by a porcine circovirus type II open reading frame (ORF) selected from the group consisting of ORFs 1, 2, 3, 4, 5, 6, 7, 8, 9, 10, 11, 12, and 13.

Another embodiment of the invention further comprises a veterinarily acceptable adjuvant and, optionally, a freeze-drying stabilizer.

In the various embodiments of the invention, the Helicobacter antigen can be an antigen of Helicobacter cerdo.

In other embodiments of the invention, the porcine circovirus type II antigen may comprise a vector that contains and expresses in vivo an antigen encoded by a porcine circovirus type II open reading frame (ORF) selected from the group consisting of ORFs 1, 2, 3, 4, 5, 6, 7, 8, 9, 10, 11, 12, and 13. In various embodiments of the invention, the vector is selected from the group consisting of a DNA plasmid, a linear DNA molecule, and a recombinant virus. In other embodiments of the invention, the recombinant virus may be selected from the group consisting of pig herpes virus, porcine adenovirus, and poxvirus.

The recombinant virus may be selected from, but is not limited to, the group consisting of Aujesky's disease virus, vaccinia virus, avipox virus, canarypox virus, and swine pox virus.

The Helicobacter antigen of the invention may be selected from the group consisting of, but not limited to, an attenuated Helicobacter strain, an inactivated Helicobacter strain, a subunit of a Helicobacter strain, and wherein the porcine circovirus antigen is selected from the group consisting of an attenuated porcine circovirus, an inactivated porcine circovirus, a subunit of porcine circovirus, and a vector that contains and expresses in vivo a nucleic acid molecule encoding the porcine circovirus antigen and is selected from the group consisting of a DNA plasmid, a linear DNA molecule, and a recombinant virus; and optionally an additional antigen of another porcine pathogen. One embodiment of the invention further comprises an additional antigen of another porcine pathogen.

In one embodiment of the invention, the additional antigen of another porcine pathogen is selected from the group consisting of: an antigen of PRRS virus, an antigen of Mycoplasma hypopneumoniae, an antigen of Actinobacillus pleuropneumoniae, an antigen of E. coli, an antigen of Atrophic Rhinitis, an antigen of Pseudorabies virus, an antigen of Hog cholera, an antigen of Swine Influenza, and combinations thereof. In another embodiment of the invention, the antigen of porcine circovirus comprises antigens of a plurality of porcine circoviruses.

Another aspect of the invention is a method for inducing an immunological response against Helicobacter strain and porcine circovirus comprising administering to a porcine an immunogenic composition.

Yet another aspect of the invention is a kit for preparing an immunogenic composition encompassing at least one Helicobacter antigen and at least one porcine circovirus antigen, wherein (i) and (ii) are packaged separately. In one embodiment of this aspect of the invention, the porcine circovirus antigen comprises at least one porcine circovirus type II antigen.

These and other aspects and features of the invention will be more fully appreciated when the following detailed description of the invention is read in conjunction with the accompanying figures.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

### BRIEF DESCRIPTION OF THE DRAWINGS

The following Detailed Description, given by way of example, but not intended to limit the invention to specific embodiments described, may be understood in conjunction with the accompanying drawings, incorporated herein by reference, in which:

FIG. 1 shows a diagram of PCVII 412, showing the location of the open reading frames.

FIGS. 2A-2C show the nucleotide sequence for the PCVII 412 genome (SEQ ID NO:1). Both senses are shown. The amino acid sequences corresponding to the translation products of the various ORFs are also shown as indicated: ORF 1 (SEQ ID NO:3); ORF 2 (SEQ ID NO:9); ORF 3 (SEQ ID NO:7); ORF 4 (SEQ ID NO:20); ORF 5 (SEQ ID NO:21); and ORF 6 (SEQ ID NO:5).

FIGS. 3A-3D show comparisons of amino acid sequences from open reading frames of PCVII 412 versus corresponding open reading frames of PCVI isolated from PK15 cells. FIG. 3A shows the amino acid sequence of ORF 1 of PCVII 412 (top line, SEQ ID NO:3) compared to the corresponding ORF from PCVI (bottom line, SEQ ID NO:4). FIG. 3B shows the amino acid sequence of ORF 6 of PCVII 412 (top line, SEQ ID NO:5) compared to the corresponding ORF from PCVI (bottom line, SEQ ID NO:6). FIG. 3C shows the amino acid sequence of ORF 3 of PCVII 412 (top line, SEQ ID NO:7) compared to the corresponding ORF from PCVI (bottom line, SEQ ID NO:8). FIG. 3D shows the amino acid sequence of ORF 2 of PCVII 412 (top line, SEQ ID NO:9) compared to the corresponding ORF from PCVI (bottom line, SEQ ID NO:10).

FIGS. 4A-4B show comparisons of the nucleotide sequences of various PCV isolates: PCVI from PK15 cells (SEQ ID NO:2), PCVII 412 (SEQ ID NO:1), PCVII 9741 (SEQ ID NO:11) and PCVII B9 (SEQ ID NO:12).

FIG. 5 shows the results of multiplex PCR used for the detection of PCV infection. The assay both identified PCV infection and distinguished between the presence of PCVI and PCVII. Lane 1 is a molecular weight marker. Lanes 2-4 are controls in the order of PCVII, PCVI and negative. Lanes 5-13 are blood samples collected from piglets from a PMWS-affected herd.

FIG. 6 shows the results of multiplex PCR conducted on various tissue samples from a PMWS-affected piglet. Lane 1 in both rows is a molecular weight marker. Lane 2 in the top row is a positive PCVII control while lane 3 is a negative control. The remaining lanes are various tissue samples collected from the PMWS-affected piglet.

FIG. 7 shows the SDS-PAGE analysis of the proteins extracted from Helicobcater cerdo and compared to H. pylori and another unidentified Helicobacter species.

FIG. 8 shows the nucleotide sequence of the porcine circovirus strain PCVII 1010 (SEQ ID NO: 24).

FIG. 9 shows a nucleotide sequence of the porcine circovirus strain PCVII 999 (SEQ ID NO: 28).

FIG. 10 shows the optimized nucleotide sequence of the porcine circovirus strain PCVII 999 (SEQ ID NO: 27).

FIG. 11 shows the nucleotide sequence of the porcine circovirus strain PCVII 1011-48121 (SEQ ID NO: 25).

FIG. 12 shows the nucleotide sequence of the porcine circovirus strain PCVII 1011-48285 (SEQ ID NO: 26).

FIG. 13 shows the nucleotide sequence of the porcine circovirus strain PCVII 1103 (SEQ ID NO: 29).

FIG. 14 shows the nucleotide sequence of the porcine circovirus strain PCVII 1121 (SEQ ID NO: 30).

### DETAILED DESCRIPTION OF THE INVENTION

The practice of the present invention will employ, unless otherwise indicated, conventional techniques of molecular biology, microbiology, recombinant DNA technology, and immunology, which are within the skill of the art. Such techniques are explained fully in the literature. See, e.g., Sambrook, Fritsch & Maniatis, Molecular Cloning: A Laboratory Manual, Vols. I, II and III, Second Edition (1989); DNA Cloning, Vols. I and II (D. N. Glover ed. 1985); Oligonucleotide Synthesis (M. J. Gait ed. 1984); Nucleic Acid Hybridization (B. D. Hames & S. J. Higgins eds. 1984); Animal Cell Culture (R. K. Freshney ed. 1986); Immobilized Cells and Enzymes (IRL press, 1986); Perbal, B., A Practical Guide to Molecular Cloning (1984); the series, Methods In Enzymology (S. Colowick and N. Kaplan eds., Academic Press, Inc.); and Handbook of Experimental Immunology, Vols. I-IV (D. M. Weir and C. C. Blackwell eds., 1986, Blackwell Scientific Publications).

Before describing the present invention in detail, it is to be understood that this invention is not limited to particular DNA, polypeptide sequences or process parameters as such may, of course, vary. It is also to be understood that the terminology used herein is for the purpose of describing particular embodiments of the invention only, and is not intended to be limiting.

The following amino acid abbreviations are used throughout the text: Alanine: Ala (A); Arginine: Arg (R); Asparagine: Asn (N); Aspartic acid: Asp (D); Cysteine: Cys (C); Glutamine: Gln (O); Glutamic acid: Glu (E); Glycine: Gly (G); Histidine: His (H); Isoleucine: Ile (I); Leucine: Leu (L); Lysine: Lys (K); Methionine: Met (M); Phenylalanine: Phe (F); Proline: Pro (P); Serine: Ser (S); Threonine: Thr (T); Tryptophan: Trp (W); Tyrosine: Tyr (Y); and Valine: Val (V).

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

In describing the present invention, the following terms will be employed and are intended to be defined as indicated below.

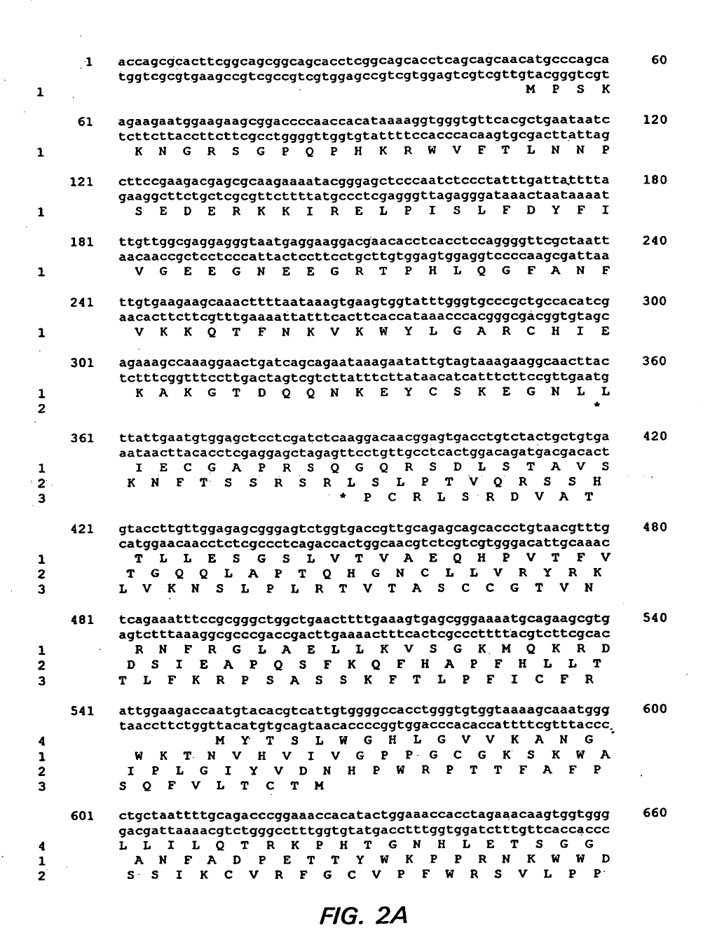

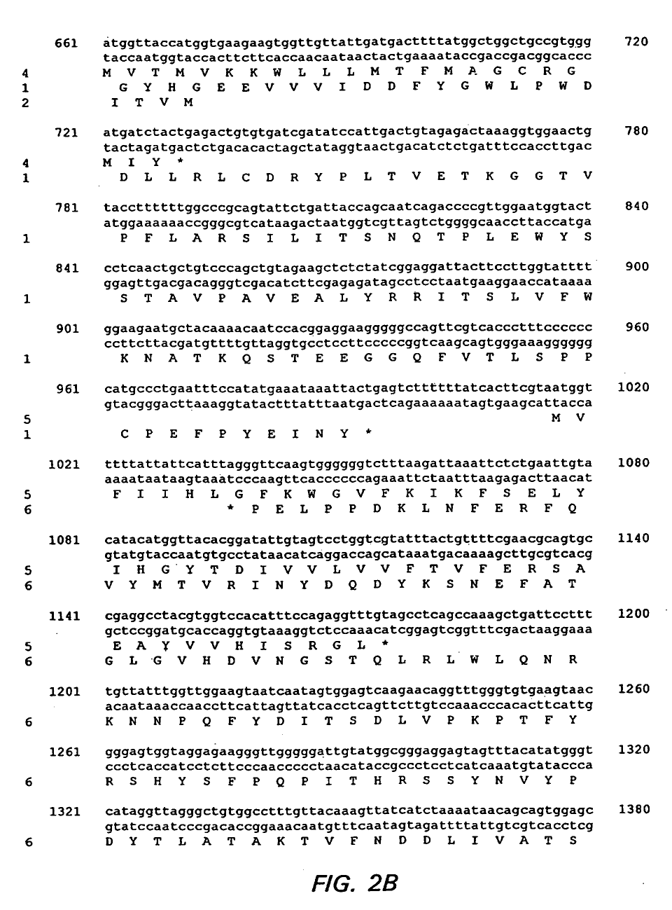

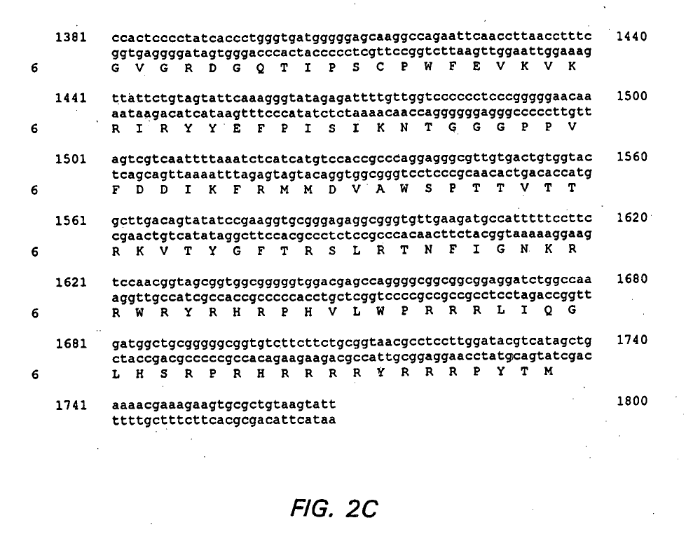

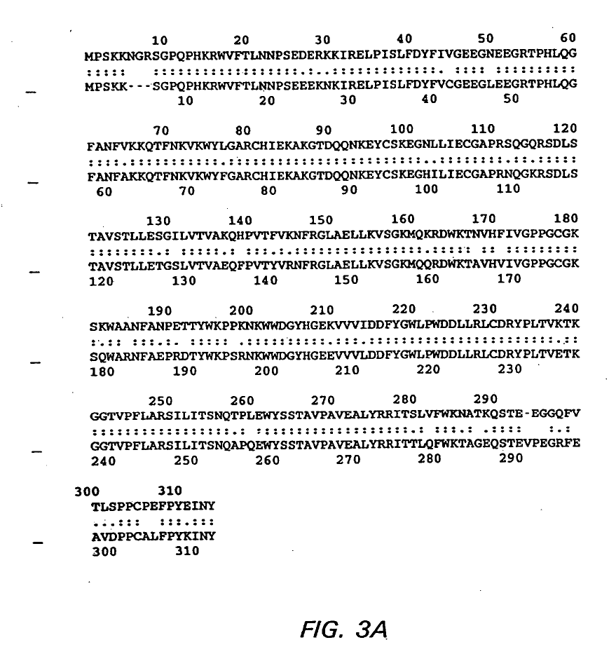

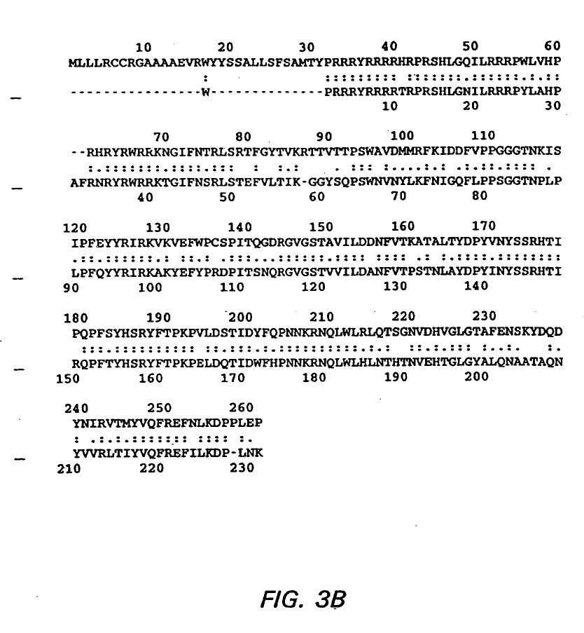

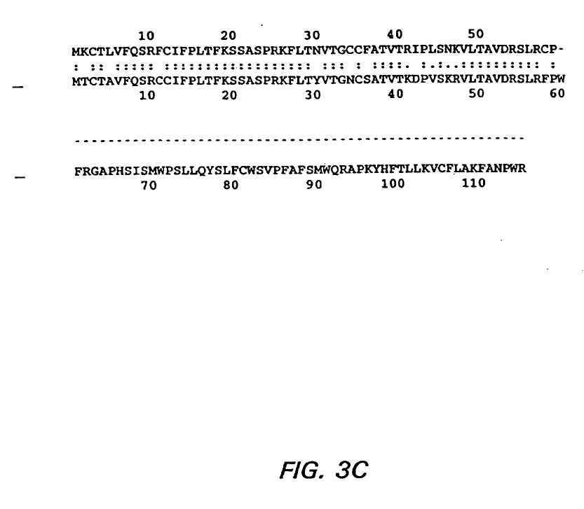

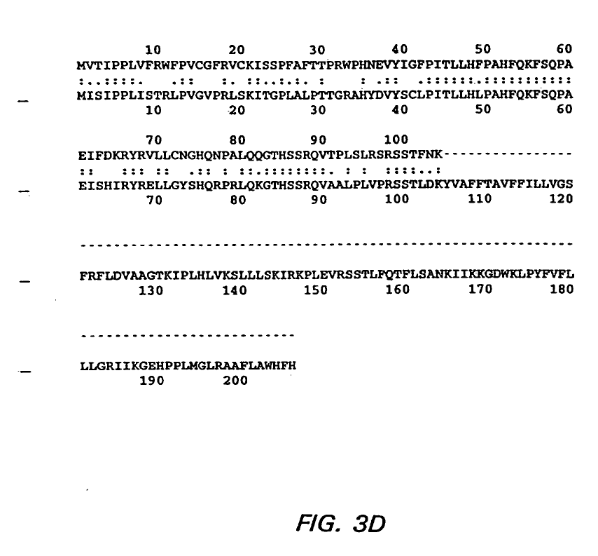

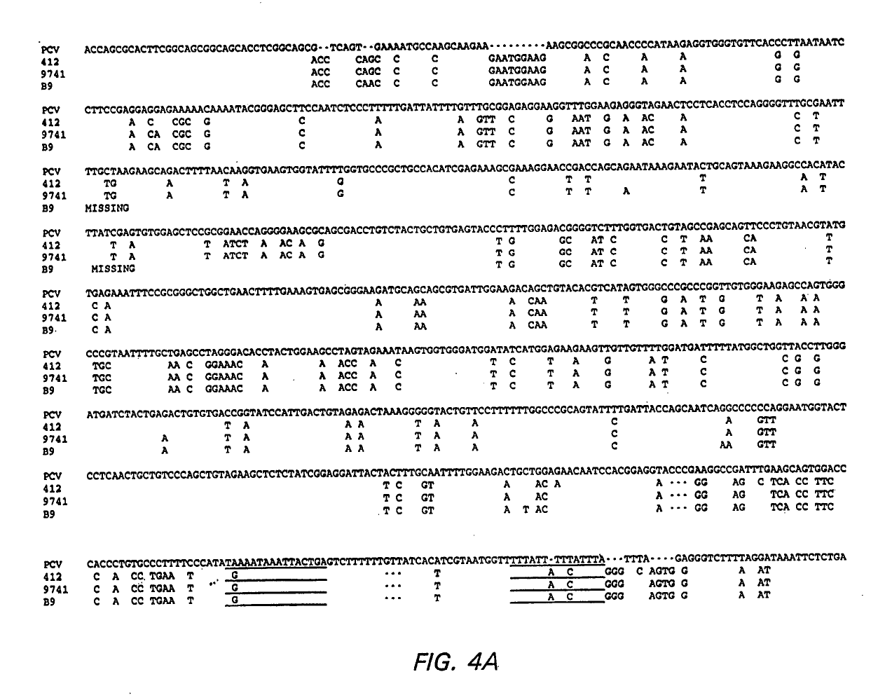

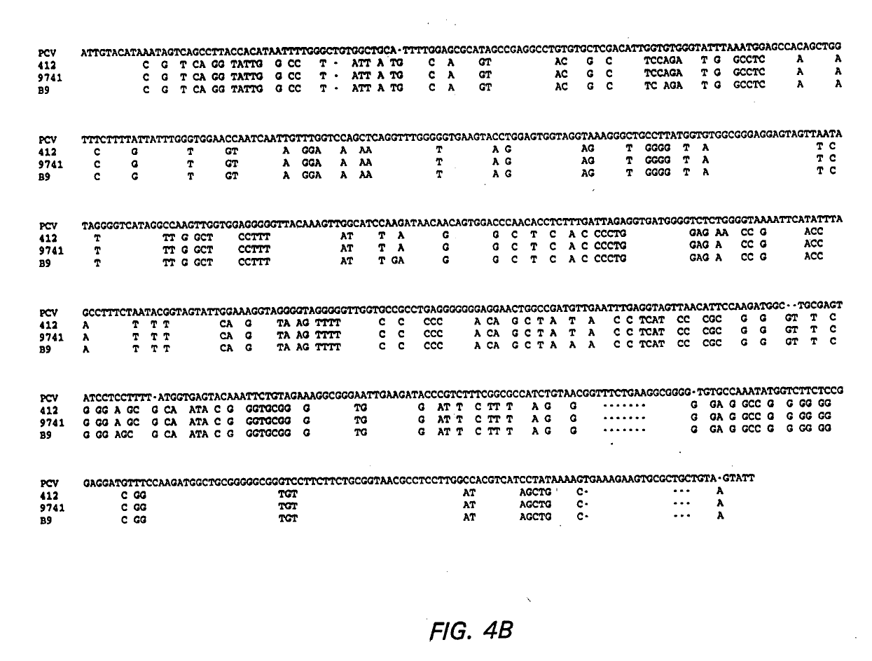

The terms “PCVII protein,” “PMWS protein” or a nucleotide sequence encoding the same, intend a protein or a nucleotide sequence, respectively, which is derived from a novel PCVII isolate, as described herein. The nucleotide sequences of several PCVII isolates are shown in FIGS. 4A-4B and the amino acid sequences corresponding to the six identified PCVII ORFs are shown in FIGS. 2A-2C. However, a PCVII or PMWS protein, or a gene encoding the same, as defined herein is not limited to the depicted sequence.

Further, as used herein, a nucleotide sequence “derived from” a PCVII genome or its complement refers to a sequence that retains the essential properties of the illustrated polynucleotide, representing a portion of the entire sequence from which it is derived, for the purpose intended. A specific, but nonlimiting, example of such derivation is represented by a sequence which encodes an identical or substantially identical amino acid sequence, but, because of codon degeneracy, utilizes different specific codons; another example is a sequence complementary to the viral DNA. A probe or oligonucleotide useful in diagnostic tests needs to retain the complementarity of the sequence shown but may be shorter than the entire sequence or may skip over portions of it. However, for use in manipulation or expression, nucleotide changes are often desirable to create or delete restriction sites, provide processing sites, or to alter the encoded amino acid sequence in ways that do not adversely affect functionality. The terms “nucleotide sequence” and “polynucleotide” refer both to ribonucleotide and a deoxyribonucleotide sequences and include both the genomic strand and its complementary sequence.

A sequence “derived from” the nucleotide sequence which comprises the genome of a PCVII isolate therefore refers to a sequence which is comprised of a sequence corresponding to a region of the genomic nucleotide sequence (or its complement), or a combination of regions of that sequence modified in ways known in the art to be consistent with its intended use. These sequences are, of course, not necessarily physically derived from the nucleotide sequence of the gene, but refer to polynucleotides generated in whatever manner which are based on the information provided by the sequence of bases in the region(s) from which the polynucleotide is derived. For example, regions from which typical DNA sequences can be “derived” include regions encoding specific epitopes. Similarly, a peptide “derived from” a PCVII ORF refers to an amino acid sequence substantially identical to that of these polypeptides or a portion thereof, having the same biological properties as that portion.

Furthermore, the derived protein or nucleotide sequences need not be physically derived from the genes described above, but may be generated in any manner, including for example, chemical synthesis, isolation (e.g., from a PCVII isolate) or by recombinant production, based on the information provided herein. Additionally, the term intends proteins having amino acid sequences substantially homologous (as defined below) to contiguous amino acid sequences encoded by the genes, which display immunological activity.

Thus, the terms intend full-length, as well as immunogenic, truncated and partial sequences, and active analogs and precursor forms of the proteins. Also included in the term are nucleotide fragments of the particular gene that include at least about 8 contiguous base pairs, more preferably at least about 10-20 contiguous base pairs, and even at least about 25 to 50 or 75 or more contiguous base pairs of the gene. Such fragments are useful as probes, in diagnostic methods, and for the recombinant production of proteins, as discussed more fully below.

The terms also include proteins in neutral form or in the form of basic or acid addition salts depending on the mode of preparation. Such acid addition salts may involve free amino groups and basic salts may be formed with free carboxyls. Pharmaceutically acceptable basic and acid addition salts are discussed further below. In addition, the proteins may be modified by combination with other biological materials such as lipids and saccharides, or by side chain modification, such as acetylation of amino groups, phosphorylation of hydroxyl side chains, oxidation of sulfhydryl groups, glycosylation of amino acid residues, as well as other modifications of the encoded primary sequence.

The term further contemplates deletions, additions and substitutions to the sequence, so long as the polypeptide functions to produce an immunological response as defined herein. In this regard, particularly preferred substitutions will generally be conservative in nature, i.e., those substitutions that take place within a family of amino acids. For example, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cystine, serine threonine, tyrosine. Phenylalanine, tryptophan, and tyrosine are sometimes classified as aromatic amino acids. It is reasonably predictable that an isolated replacement of leucine with isoleucine or valine, or vice versa; an aspartate with a glutamate or vice versa; a threonine with a serine or vice versa; or a similar conservative replacement of an amino acid with a structurally related amino acid, will not have a major effect on the biological activity. Proteins having substantially the same amino acid sequence as the reference molecule but possessing minor amino acid substitutions that do not substantially affect the immunogenicity of the protein are, therefore, within the definition of the reference polypeptide.

An “open reading frame” or “ORF” is a region of a polynucleotide sequence that encodes a polypeptide.

By “postweaning multisystemic wasting syndrome” or “PMWS” is meant a disease of vertebrate animals, in particular pigs, which is characterized clinically by progressive weight loss, tachypnea, dyspnea and jaundice. Consistent pathologic changes include lymphocytic to granulomatous interstitial pneumonia, lymphadenopathy, and, less frequently, lymphocytic to granulomatous hepatitis and nephritis. See, e.g., Clark, E. G. Proc. Am. Assoc. Swine Pract. 1997:499-501; and Harding, J. Proc. Am. Assoc. Swine Pract. 1997:503.

An “isolated” nucleic acid molecule is a nucleic acid molecule separate and discrete from the whole organism with which the molecule is found in nature; or a nucleic acid molecule devoid, in whole or part, of sequences normally associated with it in nature; or a sequence, as it exists in nature, but having heterologous sequences (as defined below) in association therewith.

The term “vaccine composition” intends any pharmaceutical composition containing an antigen, which composition can be used to prevent or treat a disease or condition in a subject. The term thus encompasses both subunit vaccines, as described below, as well as compositions containing whole killed, attenuated or inactivated microbes.

By “subunit vaccine composition” is meant a composition containing at least one immunogenic polypeptide, but not all antigens, derived from or homologous to an antigen from a pathogen of interest. Such a composition is substantially free of intact pathogen cells or particles, or the lysate of such cells or particles. Thus, a “subunit vaccine composition” is prepared from at least partially purified (preferably substantially purified) immunogenic polypeptides from the pathogen, or recombinant analogs thereof. A subunit vaccine composition can comprise the subunit antigen or antigens of interest substantially free of other antigens or polypeptides from the pathogen.

The compositions of the invention can include any pharmaceutically acceptable carrier known in the art.

The term “epitope” refers to the site on an antigen or hapten to which specific B cells and/or T cells respond. The term is also used interchangeably with “antigenic determinant” or “antigenic determinant site”. Antibodies that recognize the same epitope can be identified in a simple immunoassay showing the ability of one antibody to block the binding of another antibody to a target antigen.

An “immunological response” to a composition or vaccine is the development in the host of a cellular and/or antibody-mediated immune response to the composition or vaccine of interest. Usually, an “immunological response” includes but is not limited to one or more of the following effects: the production of antibodies, B cells, helper T cells, suppressor-T cells, and/or cytotoxic T cells and/or γδ T cells, directed specifically to an antigen or antigens included in the composition or vaccine of interest. Preferably, the host will display either a therapeutic or protective immunological response such that resistance to new infection will be enhanced and/or the clinical severity of the disease reduced. Such protection will be demonstrated by either a reduction or lack of symptoms normally displayed by an infected host, a quicker recovery time and/or a lowered viral titer in the infected host.

The terms “immunogenic” protein or polypeptide refer to an amino acid sequence which elicits an immunological response as described above. An “immunogenic” protein or polypeptide, as used herein, includes the full-length sequence of the protein, analogs thereof, or immunogenic fragments thereof. By “immunogenic fragment” is meant a fragment of a protein which includes one or more epitopes and thus elicits the immunological response described above. Such fragments can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715, all incorporated herein by reference in their entireties. Similarly, conformational epitopes are readily identified by determining spatial conformation of amino acids such as by, e.g., x-ray crystallography and 2-dimensional nuclear magnetic resonance. See, e.g., Epitope Mapping Protocols, supra.

Synthetic antigens are also included within the definition, for example, polyepitopes, flanking epitopes, and other recombinant or synthetically derived antigens. See, e.g., Bergmann et al. (1993) Eur. J. Immunol. 23:2777-2781; Bergmann et al. (1996) J. Immunol. 157:3242-3249; Suhrbier, A. (1997) Immunol. and Cell Biol. 75:402-408; Gardner et al. (1998) 12th World AIDS Conference, Geneva, Switzerland, Jun. 28-Jul. 3, 1998.

Immunogenic fragments, for purposes of the present invention, will usually include at least about 3 amino acids, preferably at least about 5 amino acids, more preferably at least about 10-15 amino acids, and most preferably 25 or more amino acids, of the molecule. There is no critical upper limit to the length of the fragment, which could comprise nearly the full-length of the protein sequence, or even a fusion protein comprising two or more epitopes of the protein.

Any of the above immunogenic proteins, immunogenic polypeptides, synthetic antigens, or immunogenic fragments can be used to raise antibodies in a host.

“Native” proteins or polypeptides refer to proteins or polypeptides isolated from the source in which the proteins naturally occur. “Recombinant” polypeptides refer to polypeptides produced by recombinant DNA techniques; i.e., produced from cells transformed by an exogenous DNA construct encoding the desired polypeptide. “Synthetic” polypeptides are those prepared by chemical synthesis.

A “vector” is a replicon, such as a plasmid, phage, or cosmid, to which another DNA segment may be attached so as to bring about the replication of the attached segment.

A DNA “coding sequence” or a “nucleotide sequence encoding” a particular protein, is a DNA sequence which is transcribed and translated into a polypeptide in vitro or in vivo when placed under the control of appropriate regulatory elements. The boundaries of the coding sequence are determined by a start codon at the 5′ (amino) terminus and a translation stop codon at the 3′ (carboxy) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A transcription termination sequence will usually be located 3′ to the coding sequence.

DNA “control elements” refers collectively to promoters, ribosome binding sites, polyadenylation signals, transcription termination sequences, upstream regulatory domains, enhancers, and the like, which collectively provide for the transcription and translation of a coding sequence in a host cell. Not all of these control sequences need always be present in a recombinant vector so long as the desired gene is capable of being transcribed and translated.

“Operably linked” refers to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter and the coding sequence and the promoter can still be considered “operably linked” to the coding sequence.

A control element, such as a promoter, “directs the transcription” of a coding sequence in a cell when RNA polymerase will bind the promoter and transcribe the coding sequence into mRNA, which is then translated into the polypeptide encoded by the coding sequence.

A “host cell” is a cell that has been transformed, or is capable of transformation, by an exogenous nucleic acid molecule.

A cell has been “transformed” by exogenous DNA when such exogenous DNA has been introduced inside the cell membrane. Exogenous DNA may or may not be integrated (covalently linked) into chromosomal DNA making up the genome of the cell. In prokaryotes and yeasts, for example, the exogenous DNA may be maintained on an episomal element, such as a plasmid. With respect to eukaryotic cells, a stably transformed cell is one in which the exogenous DNA has become integrated into the chromosome so that it is inherited by daughter cells through chromosome replication. This stability is demonstrated by the ability of the eukaryotic cell to establish cell lines or clones comprised of a population of daughter cells containing the exogenous DNA.

“Homology” refers to the percent identity between two polynucleotide or two polypeptide moieties. Two DNA, or two polypeptide sequences are “substantially homologous” to each other when the sequences exhibit at least about 80%-85%, preferably at least about 90%, and most preferably at least about 95%-98% sequence identity over a defined length of the molecules. As used herein, substantially homologous also refers to sequences showing complete identity to the specified DNA or polypeptide sequence.

Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National biomedical Research Foundation, Washington, D.C., which adapts the local homology algorithm of Smith and Waterman (1981) Advances in Appl. Math. 2:482-489 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above.

Alternatively, homology can be determined by hybridization of polynucleotides under conditions that form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

Two nucleic acid fragments are considered to be “selectively hybridizable” to a polynucleotide if they are capable of specifically hybridizing to a nucleic acid or a variant thereof (e.g., a probe that hybridizes to a PCVII nucleic acid but not to polynucleotides from other members of the circovirus family) or specifically priming a polymerase chain reaction: (i) under typical hybridization and wash conditions, as described, for example, in Sambrook et al., supra and Nucleic Acid Hybridization, supra, (ii) using reduced stringency wash conditions that allow at most about 25-30% basepair mismatches, for example: 2×SSC, 0.1% SDS, room temperature twice, 30 minutes each; then 2×SSC, 0.1% SDS, 37° C. once, 30 minutes; then 2×SSC room temperature twice, 10 minutes each, or (iii) selecting primers for use in typical polymerase chain reactions (PCR) under standard conditions (described for example, in Saiki, et al. (1988) Science 239:487-491), which result in specific amplification of sequences of PCVII or its variants.

The term “functionally equivalent” intends that the amino acid sequence of a protein is one that will elicit a substantially equivalent or enhanced immunological response, as defined above, as compared to the response elicited by a reference amino acid sequence, or an immunogenic portion thereof.

A “heterologous” region of a DNA construct is an identifiable segment of DNA within or attached to another DNA molecule that is not found in association with the other molecule in nature. Thus, when the heterologous region encodes a viral gene, the gene will usually be flanked by DNA that does not flank the viral gene in the genome of the source virus. Another example of the heterologous coding sequence is a construct where the coding sequence itself is not found in nature (e.g., synthetic sequences having codons different from the native gene). Allelic variation or naturally occurring mutational events do not give rise to a heterologous region of DNA, as used herein.

The term “treatment” as used herein refers to either (i) the prevention of infection or reinfection (prophylaxis), or (ii) the reduction or elimination of symptoms of the disease of interest (therapy).

As used herein, a “biological sample” refers to a sample of tissue or fluid isolated from a subject, including but not limited to, for example, blood, plasma, serum, fecal matter, urine, bone marrow, bile, spinal fluid, lymph tissue and lymph fluid, samples of the skin, external secretions of the skin, respiratory, intestinal, and genitourinary tracts, tears, saliva, milk, blood cells, organs, biopsies and also samples of in vitro cell culture constituents including but not limited to conditioned media resulting from the growth of cells and tissues in culture medium, e.g., recombinant cells, and cell components.

As used herein, the terms “label” and “detectable label” refer to a molecule capable of detection, including, but not limited to, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like. The term “fluorescer” refers to a substance or a portion thereof that is capable of exhibiting fluorescence in the detectable range. Particular examples of labels that may be used under the invention include fluorescein, rhodamine, dansyl, umbelliferone, Texas red, luminol, NADPH and α-β-galactosidase.

By “vertebrate subject” is meant any member of the subphylum cordata, including, without limitation, mammals such as cattle, sheep, pigs, goats, horses, and man; domestic animals such as dogs and cats; and birds, including domestic, wild and game birds such as cocks and hens including chickens, turkeys and other gallinaceous birds. The term does not denote a particular age. Thus, adult and newborn animals, as well as fetuses, are intended to be covered.

One aspect the present invention is the discovery of a new circovirus termed “PCVII” herein, isolated from PMWS-affected pigs. The useful materials and processes of the present invention are made possible by the discovery of a family of nucleotide sequences, each containing an entire genome of a novel PCVII virus. The availability of this family of polynucleotides first permits the isolation of other members of the genome family that differ by small heterogeneities. Second, it permits the construction of DNA fragments and proteins useful in diagnosis. For example, oligomers of at least about 8-10 nucleotides or more, preferably, oligomers comprising at least about 15-20 nucleotides, are useful as hybridization probes in disease diagnosis. Such probes may be used to detect the presence of the viral genome in, for example, sera of subjects suspected of harboring the virus. Similarly, the genes encoding the proteins can be cloned and used to design probes to detect and isolate homologous genes in other viral isolates.

The PCVII sequences also allow the design and production of PCVII-specific polypeptides that are useful as diagnostic reagents for the presence of antibodies raised against PCVII in serum or blood. Antibodies against these polypeptides are also useful as diagnostics. Because several open reading frames can be deciphered in the context of the complete genome, the primary structures of PCVII-related proteins can be deduced. Finally, knowledge of the gene sequences also enables the design and production of vaccines effective against PCVII and hence useful for the prevention of PMWS and also for the production of protective antibodies.

Sequencing information available from the genome allows the amino acid sequence of the various polypeptides encoded by the viral genome to be deduced and suitable epitopes identified. The full-length proteins encoded by the several ORFs identified in the PCVII genome, or suitable portions thereof, can be produced using fragments of the relevant DNA that are obtained and expressed independently, thus providing desired polypeptides using recombinant techniques. Both prokaryotic and eukaryotic hosts are useful for such expression. Short polypeptide fragments may also be chemically synthesized and linked to carrier proteins for use as vaccines. In addition, epitopes may be produced linked to a protein conferring immunogenicity. The proteins thus produced may themselves be used as vaccines, or may be used to induce immunocompetent B cells in hosts, which B cells can then be used to produce hybridomas that secrete antibodies useful in passive immunotherapy.

More particularly, the complete genetic sequences for three isolates of PCVII, PCVII 412 (SEQ ID NO:1), PCVII 9741 (SEQ ID NO:11), AND PCVII B9 (SEQ ID NO:12), are shown in FIGS. 4A-4B. The percent nucleotide sequence homologies among the various isolates of PCVII are more than 99% identical. The newly discovered viral genome shares approximately 76% identity with PCV isolated from infected PK15 cells at the nucleotide level (termed “PCVI” herein). As described further in the examples, nucleotide insertions and deletions (indels) have been found in three regions.

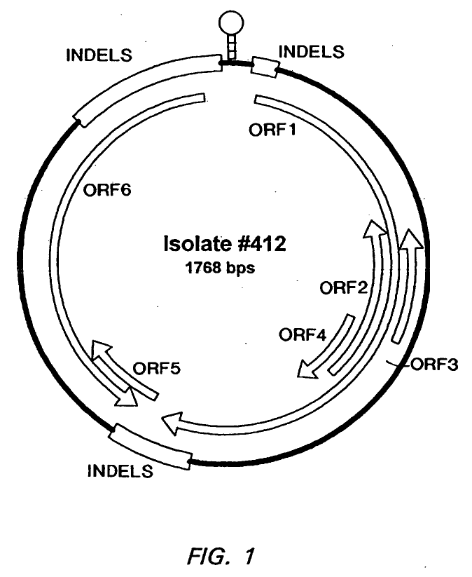

As shown in FIG. 1, the new virus contains at least six potential open reading frames (ORFs) encoding proteins comprising more than 50 amino acid residues, while PCVI derived from PK15 has seven potential ORFs. The ORFs for representative PCVII isolates occur at the following nucleotide positions, using the numbering of the PCVII isolates shown in FIGS. 4A-4B: ORF 1, 51 to 992; ORF 2, 671 to 360; ORF 3, 565 to 389; ORF 4, 553 to 729; ORF 5, 1016 to 1174; and ORF 6, 1735 to 1037. The polypeptides encoded by the six ORFs are shown in FIGS. 2A-2C.

The ORFs may be defined with respect to strain Imp1010. The invention also encompasses the use of the corresponding ORFs in any other PCVII strain, and any of the PCVII strains as defined herein or in documents cited herein. Thus, from the genomic nucleotide sequence, it is routine art to determine the ORFs using a standard software, such as MacVector®. Also, alignment of genomes with that of strain 1010 and comparison with strain 1010 ORFs allows the one skilled in the art to readily determine the ORFs on the genome for another strain (e.g. those disclosed in WO-A-99 18214, say Imp 1008, Imp 1011-48121, Imp 1011-48285, Imp 999, as well as the new strains 1103 and 1121). Using software or making alignment is not undue experimentation and directly provides access to equivalent ORFs.

Also equivalent and useful in the practice of the invention are the nucleotide sequences which change neither the functionality nor the strain specificity (say of strain type 2) of the gene considered or those of the polypeptides encoded by this gene. The sequences differing through the degeneracy of the code are, of course, be included in the practice of the invention.

The main cellular targets for PCVII are mononuclear cells in the peripheral blood, likely macrophage cells, although the virus is also found in various tissues and organs in infected animals. The affected macrophages lose their normal function, causing damage to the host immune system, leading to death.

The cloning and sequencing of the novel circoviruses has provided information about the causative agent of PMWS. As explained above, the sequencing information, as well as the clones and its gene products, are useful for diagnosis and in vaccine development. In particular, PCR and antibody-based diagnostic methods are useful in the diagnosis of the disease and were used herein to specifically identify and differentiate this novel PCVII virus from PCVI derived from persistently infected PK15 cells. The sequencing information is also useful in the design of specific primers, to express viral-specific gene products, to study the viral structure, to generate specific antibodies and to identify virulent genes in porcine circovirus-related diseases.

The new viral genomes of PCVII were obtained from viruses isolated from tissue of PMWS-affected pigs. Viral DNA was extracted from variable sources, including pellets of infected Dulac and Vero cells, peripheral blood buffy-coat cells, tissues from infected animals and serum. DNA was extracted from the samples using techniques discussed more fully in the examples.

By comparing the sequence and structural similarity among the known viruses in the circovirus family, a unique primer was designed taking advantage of the complementary sequences of a conserved stem loop structure. One-primer PCR was then performed and the products cloned. Two full-length viral genomes in different orientations inserted into a plasmid vector were completely sequenced in both directions. Additional PCR products were made and sequenced to ensure the fidelity of the primer/stem loop region.

Using similar primers, other PCVII isolates, including PCVII 9741, and PCVII B9, were obtained. The resultant sequence is provided herein, and the entire sequence, or any portion thereof, could also be prepared using synthetic methods, or by a combination of synthetic methods with retrieval of partial sequences using methods similar to those here described.

The availability of PCVII genomic sequences permits construction of expression vectors encoding viral polypeptides and antigenically active regions thereof, derived from the PCVII genome. Fragments encoding the desired proteins can be obtained from cDNA clones using conventional restriction digestion or by synthetic methods and are ligated into vectors, for example, containing portions of fusion sequences such as β-galactosidase. Any desired portion of the PCVII genome containing an open reading frame can be obtained as a recombinant protein, such as a mature or fusion protein, or can be provided by chemical synthesis or general recombinant means.

It is readily apparent that PCVII proteins encoded by the above-described DNA sequences, active fragments, analogs and chimeric proteins derived from the same, can be produced by a variety of methods. Recombinant products can take the form of partial protein sequences, full-length sequences, precursor forms that include signal sequences, mature forms without signals, or even fusion proteins (e.g., with an appropriate leader for the recombinant host, or with another subunit antigen sequence for another pathogen).

Gene libraries can be constructed and the resulting clones used to transform an appropriate host cell. Colonies can be pooled and screened using polyclonal serum or monoclonal antibodies to the PCVII protein.

Alternatively, once the amino acid sequences are determined, oligonucleotide probes that contain the codons for a portion of the determined amino acid sequences can be prepared and used to screen genomic or cDNA libraries for genes encoding the subject proteins. The basic strategies for preparing oligonucleotide probes and DNA libraries, as well as their screening by nucleic acid hybridization, are well known to those of ordinary skill in the art. See, e.g., DNA Cloning: Vol. I, supra; Nucleic Acid Hybridization, supra; Oligonucleotide Synthesis, supra; Sambrook et al., supra. Once a clone from the screened library has been identified by positive hybridization, it can be confirmed by restriction enzyme analysis and DNA sequencing that the particular library insert contains a PCVII protein gene or a homolog thereof. The genes can then be further isolated using standard techniques and, if desired, PCR approaches or restriction enzymes employed to delete portions of the full-length sequence.

Similarly, genes can be isolated directly from viruses using known techniques, such as phenol extraction and the sequence further manipulated to produce any desired alterations. See, e.g., the examples herein and Hamel et al. (1998) J. Virol. 72:5262-5267, for a description of techniques used to obtain and isolate viral DNA.

Alternatively, DNA sequences can be prepared synthetically rather than cloned. The DNA sequences can be designed with the appropriate codons for the particular amino acid sequence if the sequences are to be used in protein production. In general, one will select preferred codons for the intended host if the sequence will be used for expression. The complete sequence is assembled from overlapping oligonucleotides prepared by standard methods and assembled into a complete coding sequence. See, e.g., Edge (1981) Nature 292:756; Nambair et al. (1984) Science 223: 1299; Jay et al. (1984) J. Biol. Chem. 259:6311.

Once coding sequences for the desired proteins have been prepared or isolated, they can be cloned into any suitable vector or replicon. Numerous cloning vectors are known to those of skill in the art, and the selection of an appropriate cloning vector is a matter of choice. Examples of recombinant DNA vectors for cloning and host cells which they can transform include the bacteriophage λ (E. coli), pBR322 (E. coli), pACYC177 (E. coli), pKT230 (gram-negative bacteria), pGV1106 (gram-negative bacteria), pLAFR1 (gram-negative bacteria), pME290 (non-E. coli gram-negative bacteria), pHV14 (E. coli and Bacillus subtilis), pBD9 (Bacillus), pU61 (Streptomyces), pUC6 (Streptomyces), YIp5 (Saccharomyces), YCp19 (Saccharomyces) and bovine papilloma virus (mammalian cells). See, Sambrook et al., supra; DNA Cloning, supra; B. Perbal, supra.

The gene can be placed under the control of a promoter, ribosome binding site (for bacterial expression) and, optionally, an operator (collectively referred to herein as “control” elements), so that the DNA sequence encoding the desired protein is transcribed into RNA in the host cell transformed by a vector containing this expression construction. The coding sequence may or may not contain a signal peptide or leader sequence. If a signal sequence is included, it can either be the native, homologous sequence, or a heterologous sequence. Leader sequences can be removed by the host in post-translational processing. See, e.g., U.S. Pat. Nos. 4,431,739; 4,425,437; 4,338,397.

Other regulatory sequences may also be desirable which allow for regulation of expression of the protein sequences relative to the growth of the host cell. Regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences.

The control sequences and other regulatory sequences may be ligated to the coding sequence prior to insertion into a vector, such as the cloning vectors described above. Alternatively, the coding sequence can be cloned directly into an expression vector which already contains the control sequences and an appropriate restriction site.

In some cases it may be necessary to modify the coding sequence so that it may be attached to the control sequences with the appropriate orientation; i.e., to maintain the proper reading frame. It may also be desirable to produce mutants or analogs of the desired PCVII protein. Mutants or analogs may be prepared by the deletion of a portion of the sequence encoding the protein, by insertion of a sequence, and/or by substitution of one or more nucleotides within the sequence. Techniques for modifying nucleotide sequences, such as site-directed mutagenesis, are described in, e.g., Sambrook et al., supra; DNA Cloning, supra; Nucleic Acid Hybridization, supra.

The expression vector is then used to transform an appropriate host cell. A number of mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human hepatocellular carcinoma cells (e.g., Hep G2), Mandin-Darby bovine kidney (“MDBK”) cells, as well as others. Similarly, bacterial hosts such as E. coli, Bacillus subtilis, and Streptococcus spp., will find use with the present expression constructs. Yeast hosts useful in the present invention include inter alia, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, inter alia, Aedes aegypti, Autographa californica, Bombyx mori, Drosophila melanogaster, Spodoptera frugiperda, and Trichoplusia ni.

Depending on the expression system and host selected, the proteins of the present invention are produced by culturing host cells transformed by an expression vector described above under conditions whereby the protein of interest is expressed. The protein is then isolated from the host cells and purified. If the expression system secretes the protein into the growth media, the protein can be purified directly from the media. If the protein is not secreted, it is isolated from cell lysates. The selection of the appropriate growth conditions and recovery methods are within the skill of the art.

The proteins of the present invention may also be produced by chemical synthesis such as solid phase peptide synthesis, using known amino acid sequences or amino acid sequences derived from the DNA sequence of the genes of interest. Such methods are known to those skilled in the art. See, e.g., J. M. Stewart and J. D. Young, Solid Phase Peptide Synthesis, 2nd Ed., Pierce Chemical Co., Rockford, Ill. (1984) and G. Barany and R. B. Merrifield, The Peptides: Analysis, Synthesis, Biology, editors E. Gross and J. Meienhofer, Vol. 2, Academic Press, New York, (1980), pp. 3-254, for solid phase peptide synthesis techniques; and M. Bodansky, Principles of Peptide Synthesis, Springer-Verlag, Berlin (1984) and E. Gross and J. Meienhofer, Eds., The Peptides: Analysis, Synthesis, Biology, supra, Vol. 1, for classical solution synthesis. Chemical synthesis of peptides may be preferable if a small fragment of the antigen in question is capable of raising an immunological response in the subject of interest.

Analysis of the genome shows the presence of at least six open reading frames, at least one of which encodes the putative DNA replicase gene.

The antigenic region of peptides is generally relatively small, typically 10 amino acids or less in length. Fragments of as few as 5 amino acids may typically characterize an antigenic region. Accordingly, using the genome of PCVII as a basis, DNAs encoding short segments of polypeptides, derived from any of the various ORFs of PCVII, such as ORFs 1-6, and particularly ORF 6, can be expressed recombinantly either as fusion proteins or as isolated peptides. In addition, short amino acid sequences can be chemically synthesized. In instances wherein the synthesized peptide is correctly configured so as to provide the correct epitope, but too small to be immunogenic, the peptide may be linked to a suitable carrier.

A number of techniques for obtaining such linkage are known in the art, including the formation of disulfide linkages using N-succinimidyl-3-(2-pyridyl-thio)propionate (SPDP) and succinimidyl 4-(N-maleimido-methyl)cyclohexane-1-carboxylate (SMCC) obtained from Pierce Company, Rockford, Ill. (If the peptide lacks a sulfhydryl, this can be provided by addition of a cysteine residue.) These reagents create a disulfide linkage between themselves and peptide cysteine residues on one protein and an amide linkage through the e-amino on a lysine, or other free amino group in the other. A variety of such disulfide/amide-forming agents are known. See, for example, Immun. Rev. (1982) 62:185. Other bifunctional coupling agents form a thioether rather than a disulfide linkage. Many of these thioether-forming agents are commercially available and include reactive esters of 6-maleimidocaproic acid, 2-bromoacetic acid, 2-iodoacetic acid, 4-(N-maleimido-methyl)cycloh-exane-1-carboxylic acid, and the like. The carboxyl groups can be activated by combining them with succinimide or 1-hydroxy-2-nitro-4-sulfonic acid, sodium salt. The foregoing list is not meant to exhaustive, and modifications of the named compounds can clearly be used.

Any carrier may be used, which does not itself induce the production of antibodies harmful to the host, such as the various serum albumins, tetanus toxoids, or keyhole limpet hemocyanin (KLH).

The conjugates, when injected into suitable subjects, will result in the production of antisera which contain immunoglobulins specifically reactive against not only the conjugates, but also against fusion proteins carrying the analogous portions of the sequence, and against appropriate determinants within whole PCVII.

Proteins encoded by the novel viruses of the present invention, or their fragments, can be used to produce antibodies, both polyclonal and monoclonal. If polyclonal antibodies are desired, a selected mammal, (e.g., mouse, rabbit, goat, horse, etc.) is immunized with an antigen of the present invention, or its fragment, or a mutated antigen. Serum from the immunized animal is collected and treated according to known procedures. See, e.g., Jurgens et al. (1985) J. Chrom. 348:363-370. If serum containing polyclonal antibodies is used, the polyclonal antibodies can be purified by immunoaffinity chromatography, using known procedures.

Monoclonal antibodies to the proteins and to the fragments thereof, can also be readily produced by one skilled in the art. The general methodology for making monoclonal antibodies by using hybridoma technology is well known. Immortal antibody-producing cell lines can be created by cell fusion, and also by other techniques such as direct transformation of B lymphocytes with oncogenic DNA, or transfection with Epstein-Barr virus. See, e.g., M. Schreier et al., Hybridoma Techniques (1980); Hammerling et al., Monoclonal Antibodies and T-cell Hybridomas (1981); Kennett et al., Monoclonal Antibodies (1980); see also U.S. Pat. Nos. 4,341,761; 4,399,121; 4,427,783; 4,444,887; 4,452,570; 4,466,917; 4,472,500, 4,491,632; and 4,493,890. Panels of monoclonal antibodies produced against the desired protein, or fragment thereof, can be screened for various properties; i.e., for isotype, epitope, affinity, etc. Monoclonal antibodies are useful in purification, using immunoaffinity techniques, of the individual antigens which they are directed against. Both polyclonal and monoclonal antibodies can also be used for passive immunization or can be combined with subunit vaccine preparations to enhance the immune response. Polyclonal and monoclonal antibodies are also useful for diagnostic purposes.

The novel viral proteins of the present invention can be formulated into vaccine compositions, either alone or in combination with other antigens, for use in immunizing subjects as described below. Methods of preparing such formulations are described in, e.g., Remington's Pharmaceutical Sciences, Mack Publishing Company, Easton, Pa., 18 Edition, 1990. Typically, the vaccines of the present invention are prepared as injectables, either as liquid solutions or suspensions. Solid forms suitable for solution in or suspension in liquid vehicles prior to injection may also be prepared. The preparation may also be emulsified or the active ingredient encapsulated in liposome vehicles. The active immunogenic ingredient is generally mixed with a compatible pharmaceutical vehicle, such as, for example, water, saline, dextrose, glycerol, ethanol, or the like, and combinations thereof. In addition, if desired, the vehicle may contain minor amounts of auxiliary substances such as wetting or emulsifying agents and pH buffering agents.

Adjuvants that enhance the effectiveness of the vaccine may also be added to the formulation. Such adjuvants include, without limitation, adjuvants formed from aluminum salts (alum), such as aluminum hydroxide, aluminum phosphate, aluminum sulfate, etc; oil-in-water and water-in-oil emulsion formulations, such as Complete Freunds Adjuvant (CFA), Incomplete Freunds Adjuvant (IFA), pyridine and dimethyldioctadecyl ammonium bromide (DDA); adjuvants formed from bacterial cell wall components such as adjuvants including monophosphoryl lipid A (MPL) (Imoto et al. (1985) Tet. Lett. 26:1545-1548), trehalose dimycolate (TDM), and cell wall skeleton (CWS); adjuvants derived from ADP-ribosylating bacterial toxins, such as derived from diphtheria toxin (for example, CRM₁₉₇, a non-toxic diphtheria toxin mutant (see, e.g., Bixler et al. (1989) Adv. Exp. Med. Biol. 251:175; and Constantino et al. (1992) Vaccine), pertussis toxin (PT), cholera toxin (CT), the E. coli heat-labile toxins (LT1 and LT2), Pseudomonas endotoxin A, C. botulinum C2 and C3 toxins, as well as toxins from C. perfringens, C. spiriforma and C. difficile; saponin adjuvants such as Quil A (U.S. Pat. No. 5,057,540), or particles generated from saponins such as ISCOMs (immunostimulating complexes); cytokines, such as interleukins (e.g., IL-1, IL-2, IL-4, IL-5, IL-6, IL-7, IL-12, etc.), interferons (e.g., γ-interferon), macrophage colony stimulating factor (M-CSF), tumor necrosis factor (TNF), etc; muramyl peptides such as N-acetyl-muramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′-dipalmitoyl-sn-glycero-3 huydroxyphosphoryloxy)-ethylamine (MTP-PE), etc.; adjuvants derived from the CpG family of molecules, CpG dinucleotides and synthetic oligonucleotides which comprise CpG motifs (see, e.g., Krieg et al. Nature (1995) 374:546 and Davis et al. J. Immunol. (1998) 160:870-876); and synthetic adjuvants such as PCPP (Poly di(carboxylatophenoxy)phosphazene) (Payne et al. Vaccines (1998) 16:92-98). Such adjuvants are commercially available from a number of distributors such as Accurate Chemicals; Ribi Immunechemicals, Hamilton, Mont.; GIBCO; Sigma, St. Louis, Mo.

As explained above, the proteins may be linked to a carrier in order to increase the immunogenicity thereof. Suitable carriers include large, slowly metabolized macromolecules such as proteins, including serum albumins, keyhole limpet hemocyanin, immunoglobulin molecules, thyroglobulin, ovalbumin, and other proteins well known to those skilled in the art; polysaccharides, such as sepharose, agarose, cellulose, cellulose beads and the like; polymeric amino acids such as polyglutamic acid, polylysine, and the like; amino acid copolymers; and inactive virus particles.

The proteins may be used in their native form or their functional group content may be modified by, for example, succinylation of lysine residues or reaction with Cys-thiolactone. A sulfhydryl group may also be incorporated into the carrier (or antigen) by, for example, reaction of amino functions with 2-iminothiolane or the N-hydroxysuccinimide ester of 3-(4-dithiopyridyl propionate. Suitable carriers may also be modified to incorporate spacer arms (such as hexamethylene diamine or other bifunctional molecules of similar size) for attachment of peptides.

Other suitable carriers for the proteins of the present invention include VP6 polypeptides of rotaviruses, or functional fragments thereof, as disclosed in U.S. Pat. No. 5,071,651, incorporated herein by reference. Also useful is a fusion product of a viral protein and the subject immunogens made by methods disclosed in U.S. Pat. No. 4,722,840. Still other suitable carriers include cells, such as lymphocytes, since presentation in this form mimics the natural mode of presentation in the subject, which gives rise to the immunized state. Alternatively, the proteins of the present invention may be coupled to erythrocytes, preferably the subject's own erythrocytes. Methods of coupling peptides to proteins or cells are known to those of skill in the art.

Furthermore, the proteins may be formulated into vaccine compositions in either neutral or salt forms. Pharmaceutically acceptable salts include the acid addition salts (formed with the free amino groups of the active polypeptides) and which are formed with in-organic acids such as, for example, hydrochloric or phosphoric acids, or such organic acids as acetic, oxalic, tartaric, mandelic, and the like. Salts formed from free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Vaccine formulations will contain a “therapeutically effective amount” of the active ingredient, that is, an amount capable of eliciting an immune response in a subject to which the composition is administered. Such a response will be demonstrated by either a reduction or lack of symptoms normally displayed by an infected host and/or a quicker recovery time.

The exact amount is readily determined by one skilled in the art using standard tests. The protein concentration will typically range from about 1% to about 95% (w/w) of the composition, or even higher or lower if appropriate.

To immunize a subject, the vaccine is generally administered parenterally, usually by intramuscular injection. Other modes of administration, however, such as subcutaneous, intraperitoneal and intravenous injection, are also acceptable. The quantity to be administered depends on the animal to be treated, the capacity of the animal's immune system to synthesize antibodies, and the degree of protection desired. Effective dosages can be readily established by one of ordinary skill in the art through routine trials establishing dose response curves. The subject is immunized by administration of the vaccine in at least one dose, and preferably two doses. Moreover, the animal may be administered as many doses as is required to maintain a state of immunity to infection.

Additional vaccine formulations which are suitable for other modes of administration include suppositories and, in some cases, aerosol, intranasal, oral formulations, and sustained release formulations. For suppositories, the vehicle composition will include traditional binders and carriers, such as, polyalkaline glycols, or triglycerides. Such suppositories may be formed from mixtures containing the active ingredient in the range of about 0.5% to about 10% (w/w), preferably about 1% to about 2%. Oral vehicles include such normally employed excipients as, for example, pharmaceutical grades of mannitol, lactose, starch, magnesium, stearate, sodium saccharin cellulose, magnesium carbonate, and the like. These oral vaccine compositions may be taken in the form of solutions, suspensions, tablets, pills, capsules, sustained release formulations, or powders, and contain from about 10% to about 95% of the active ingredient, preferably about 25% to about 70%.

Intranasal formulations will usually include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject proteins by the nasal mucosa.

Controlled or sustained release formulations are made by incorporating the protein into carriers or vehicles such as liposomes, nonresorbable impermeable polymers such as ethylenevinyl acetate copolymers and Hytrel® copolymers, swellable polymers such as hydrogels, or resorbable polymers such as collagen and certain polyacids or polyesters such as those used to make resorbable sutures. The proteins can also be delivered using implanted mini-pumps, well known in the art.

The proteins of the instant invention can also be administered via a carrier virus that expresses the same. Carrier viruses that will find use with the instant invention include but are not limited to the vaccinia and other poxviruses, adenovirus, and herpes virus. By way of example, vaccinia virus recombinants expressing the novel proteins can be constructed as follows. The DNA encoding the particular protein is first inserted into an appropriate vector so that it is adjacent to a vaccinia promoter and flanking vaccinia DNA sequences, such as the sequence encoding thymidine kinase (TK). This vector is then used to transfect cells that are simultaneously infected with vaccinia. Homologous recombination serves to insert the vaccinia promoter plus the gene encoding the instant protein into the viral genome. Culturing the cells in the presence of 5-bromodeoxyuridine and picking viral plaques resistant thereto can select the resulting TK-recombinant.

An alternative route of administration involves gene therapy or nucleic acid immunization. Thus, nucleotide sequences (and accompanying regulatory elements) encoding the subject proteins can be administered directly to a subject for in vivo translation thereof. Alternatively, transfecting the subject's cells or tissues ex vivo and reintroducing the transformed material into the host can accomplish gene transfer. DNA can be directly introduced into the host organism, i.e., by injection (see U.S. Pat. Nos. 5,580,859 and 5,589,466; International Publication No. WO 90/11092; and Wolff et al. (1990) Science 247:1465-1468). Liposome-mediated gene transfer can also be accomplished using known methods. See, e.g., U.S. Pat. No. 5,703,055; Hazinski et al. (1991) Am. J. Respir. Cell Mol. Biol. 4:206-209; Brigham et al. (1989) Am. J. Med. Sci. 298:278-281; Canonico et al. (1991) Clin. Res. 39:219A; and Nabel et al. (1990) Science 249:1285-1288. Targeting agents, such as antibodies directed against surface antigens expressed on specific cell types, can be covalently conjugated to the liposomal surface so that the nucleic acid can be delivered to specific tissues and cells susceptible to infection.

As explained above, the proteins of the present invention may also be used as diagnostics to detect the presence of reactive antibodies of PCVII in a biological sample in order to determine the presence of PCVII infection. For example, the presence of antibodies reactive with the proteins can be detected using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, etc. The reactions generally include revealing labels such as fluorescent, chemiluminescent, radioactive, enzymatic labels or dye molecules, or other methods for detecting the formation of a complex between the antigen and the antibody or antibodies reacted therewith.

The aforementioned assays generally involve separation of unbound antibody in a liquid phase from a solid phase support to which antigen-antibody complexes are bound. Solid supports that can be used in the practice of the invention include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, and the like.

Typically, a solid support is first reacted with a solid phase component (e.g., one or more PCVII proteins) under suitable binding conditions such that the component is sufficiently immobilized to the support. First coupling the antigen to a protein with better binding properties can sometimes enhance immobilization of the antigen to the support. Suitable coupling proteins include, but are not limited to, macromolecules such as serum albumins including bovine serum albumin (BSA), keyhole limpet hemocyanin, immunoglobulin molecules, thyroglobulin, ovalbumin, and other proteins well known to those skilled in the art. Other molecules that can be used to bind the antigens to the support include polysaccharides, polylactic acids, polyglycolic acids, polymeric amino acids, amino acid copolymers, and the like. Such molecules and methods of coupling these molecules to the antigens, are well known to those of ordinary skill in the art. See, e.g., Brinkley, M. A. Bioconjugate Chem. (1992) 3:2-13; Hashida et al., J. Appl. Biochem. (1984) 6:56-63; and Anjaneyulu and Staros, International J. of Peptide and Protein Res. (1987) 30:117-124.

After reacting the solid support with the solid phase component, any non-immobilized solid-phase components are removed from the support by washing, and the support-bound component is then contacted with a biological sample suspected of containing ligand moieties (e.g., antibodies toward the immobilized antigens) under suitable binding conditions. After washing to remove any non-bound ligand, a secondary binder moiety is added under suitable binding conditions, wherein the secondary binder is capable of associating selectively with the bound ligand. The presence of the secondary binder can then be detected using techniques well known in the art.

More particularly, an ELISA method can be used, wherein the wells of a microtiter plate are coated with a desired protein. A biological sample containing or suspected of containing anti-protein immunoglobulin molecules is then added to the coated wells. After a period of incubation sufficient to allow antibody binding to the immobilized antigen, the plate(s) can be washed to remove unbound moieties and a detectably labeled secondary binding molecule added. The secondary binding molecule is allowed to react with any captured sample antibodies, the plate washed and the presence of the secondary binding molecule detected using methods well known in the art.

Thus, in one particular embodiment, the presence of bound anti-antigen ligands from a biological sample can be readily detected using a secondary binder comprising an antibody directed against the antibody ligands. A number of anti-porcine immunoglobulin (Ig) molecules are known in the art that can be readily conjugated to a detectable enzyme label, such as horseradish peroxidase, alkaline phosphatase or urease, using methods known to those of skill in the art. An appropriate enzyme substrate is then used to generate a detectable signal. In other related embodiments, competitive-type ELISA techniques can be practiced using methods known to those skilled in the art.

Assays can also be conducted in solution, such that the proteins and antibodies specific for those proteins form complexes under precipitating conditions. In one particular embodiment, proteins can be attached to a solid phase particle (e.g., an agarose bead or the like) using coupling techniques known in the art, such as by direct chemical or indirect coupling. The antigen-coated particle is then contacted under suitable binding conditions with a biological sample suspected of containing antibodies for the proteins. Cross-linking between bound antibodies causes the formation of particle-antigen-antibody complex aggregates that can be precipitated and separated from the sample using washing and/or centrifugation. The reaction mixture can be analyzed to determine the presence or absence of antibody-antigen complexes using any of a number of standard methods, such as those immunodiagnostic methods described above.

In yet a further embodiment, an immunoaffinity matrix can be provided, wherein a polyclonal population of antibodies from a biological sample suspected of containing antibodies to the protein of interest is immobilized to a substrate. In this regard, an initial affinity purification of the sample can be carried out using immobilized antigens. The resultant sample preparation will thus only contain anti-PCVII moieties, avoiding potential nonspecific binding properties in the affinity support. A number of methods of immobilizing immunoglobulins (either intact or in specific fragments) at high yield and good retention of antigen binding activity are known in the art. Not being limited by any particular method, immobilized protein A or protein G can be used to immobilize immunoglobulins.

Accordingly, once the immunoglobulin molecules have been immobilized to provide an immunoaffinity matrix, labeled proteins are contacted with the bound antibodies under suitable binding conditions. After any non-specifically bound antigen has been washed from the immunoaffinity support, the presence of bound antigen can be determined by assaying for label using methods known in the art.

Additionally, antibodies raised to the proteins, rather than the proteins themselves, can be used in the above-described assays in order to detect the presence of antibodies to the proteins in a given sample. These assays are performed essentially as described above and are well known to those of skill in the art.

Furthermore, nucleic acid-based assays may also be conducted. In this regard, using the disclosed PCVII nucleic acid sequences as a basis, oligomers can be prepared which are useful as hybridization probes or PCR primers to detect the presence of the viral genome in, for example, biological samples from subjects suspected of harboring the virus. Oligomers for use in this embodiment of the invention are approximately 8 nucleotides or more in length, preferably at least about 10-12 nucleotides in length, more preferably at least about 15 to 20 nucleotides in length and up to 50 or more nucleotides in length. Preferably, the oligomers derive from regions of the viral genome that lack heterogeneity.

The oligomers are prepared either by excision from the genome, or recombinantly or synthetically. For example, the oligomers can be prepared using routine methods, such automated oligonucleotide synthetic methods.

The oligomers may be used as probes in diagnostic assays. In a representative assay, the biological sample to be analyzed is treated to extract the nucleic acids contained therein. The resulting nucleic acid from the sample may be subjected to gel electrophoresis or other size separation techniques. Alternatively, the nucleic acid sample may be dot-blotted without size separation. The probes are then labeled with a reporter moiety. Suitable labels, and methods for labeling probes, are known in the art and include, for example, radioactive labels incorporated by nick translation or kinasing, biotin, fluorescent probes and chemiluminescent probes. The nucleic acids extracted from the sample are then treated with the labeled probe under hybridization conditions of suitable stringencies.

The probes can be made completely complementary to the targeted PCVII gene sequence. However, when longer probes are used in the diagnostic assays, the amount of complementarity may be less. Generally, conditions of high stringency are used in the assay methods, especially if the probes are completely or highly complementary. However, lower stringency conditions should be used when targeting regions of heterogeneity. Methods of adjusting stringency are well known in the art. Such adjustments are made during hybridization and the washing procedure and include adjustments to temperature, ionic strength, concentration of formamide and length of time of the reaction. These factors are outlined in, e.g., Sambrook et al., supra.

In a more specific embodiment, the above-described method includes the use of PCVII nucleic acid specific probes where two probes (primers) define an internal region of the PCVII genome. In this embodiment, each probe has one strand containing a 3′-end internal to the PCVII nucleic acid internal region. The nucleic acid/probe hybridization complexes are then converted to double-strand probes containing fragments by primer extension reactions. Probe-containing fragments are amplified by successively repeating the steps of (i) denaturing the double-stranded fragments to produce single-stranded fragments, (ii) hybridizing the single strands with the probes to form strand/probe complexes, (iii) generating double-stranded fragments from the strand/probe complexes in the presence of DNA polymerase and all four deoxyribonucleotides, and (iv) repeating steps (i) to (iii) until a desired degree of amplification has been achieved. Amplification products are then identified according to established procedures. The method of the invention may further include a third polynucleotide probe capable of selectively hybridizing to the internal region described above but not to the specific probe/primer sequences used for amplification.

PCR techniques, such as those described above, are well known in the art. See, e.g., PCR Protocols: A Guide to Methods and Applications (Academic Press); PCR A Practical Approach (IRL press); Saiki et al. (1986) Nature 324:163.

Other amplification methods can also be used in the nucleic acid-based assays, such as ligase chain reaction (LCR), PCR, Q-beta replicase, and the like.

Other assays for use herein include the “Bio-Bridge” system which uses terminal deoxynucleotide transferase to add unmodified 3′-poly-dT-tails to a nucleic acid probe (Enzo Biochem. Corp.). The poly dt-tailed probe is hybridized to the target nucleotide sequence, and then to a biotin-modified poly-A. Additionally, EP 124221 describes a DNA hybridization assay wherein the analyte is annealed to a single-stranded DNA probe that is complementary to an enzyme-labelled oligonucleotide, and the resulting tailed duplex is hybridized to an enzyme-labelled oligonucleotide. EP 204510 describes a DNA hybridization assay in which analyte DNA is contacted with a probe that has a tail, such as a poly-dT-tail, an amplifier strand that has a sequence that hybridizes to the tail of the probe, such as a poly-A sequence, and which is capable of binding a plurality of labelled strands. The technique first may involve amplification of the target PCVII sequences in sera to approximately 10⁶ sequences/ml, as described above. The amplified sequence(s) then may be detected using a hybridization assay known in the art.

Furthermore, nucleic acid sequences derived from the PCVII viral genome may also be used for in situ hybridization assays. Generally, such assays employ formalin-fixed cell culture preparations or tissues, such as lymph node, spleen, tonsil, liver, lung, heart, kidney, pancreas, nasal turbinate, large and small intestine, and the like. See, e.g., Sirinarumitr et al. (1996) J. Virol. Meth. 56:149-160, for a description of a suitable in situ hybridization assay.

The above-described assay reagents, including the proteins, antibodies thereto or oligomers can be provided in kits, with suitable instructions and other necessary reagents, in order to conduct immunoassays as described above. The kit can also contain, depending on the particular immunoassay used, suitable labels and other packaged reagents and materials (i.e. wash buffers and the like). Standard immunoassays, such as those described above, can be conducted using these kits.

The applicant has also succeeded in isolating five new PCV strains from pulmonary or ganglionic samples obtained from farms situated in Canada, the United States (California) and France (Brittany). These viruses have been detected in lesions in pigs with the PMWS syndrome, but not in healthy pigs. In addition, novel species of bacteria of the genus Helicobacter have been identified and found to be resident in and on the mucosal surfaces of the stomach and esophagus of pigs suffering from gastro-esophageal ulceration. One novel Helicobacter species is H. cerdo, which is closely related to, but not identical with, H. pylori.

The applicant has, in addition, sequenced the genomes of four of the new PCV strains, namely the strains obtained from Canada and the United States as well as two French strains. The strains exhibit a very strong homology with each other at the nucleotide level, exceeding 96% and much weaker with the PK15 strain, about 76%. The new strains can thus be considered as being representative of a new type of porcine circovirus, called here type II, type I being represented by PK15.

Purified preparations of the five strains were deposited under the Budapest Treaty at the ECACC (European Collection of Cell Cultures, Centre for Applied Microbiology & Research, Porton Down, Salisbury, Wiltshire SP4 OJG, United Kingdom) on Thursday Oct. 2, 1997 as accession Nos. V97100219 (called here Imp.1008PCV); V97100218 (called here Imp.1010PCV); V97100217 (called here Imp.999PCV); and, on Friday Jan. 16, 1998: accession Nos. V98011608 (called here Imp.1011-48285); and V98011609 (called here Imp.1011-48121).

PCVII strains were also isolated from a litter of aborted piglets from a farm experiencing late term abortions and stillbirths. Severe, diffuse myocarditis was present in one piglet associated with extensive immunohistochemical staining for PCVII antigen. Variable amounts of PCVII antigen were also present in liver, lung and kidney of multiple fetuses. The presence of other agents that have been associated with fetal lesions and abortion in swine including porcine parvovirus, porcine reproductive respiratory syndrome virus, encephalomyocarditis virus and enterovirus could not be established.

Tissues obtained from 30 high health herds over a four-year period, and tested in routine cases of abortion or reproductive failure, were positive for PCVII in two submissions involving several stillborn piglets and non-viable neonates presenting with severe diffuse myocarditis, cardiac hypertrophy and evidence of chronic passive congestion. The two positive submissions were from the same farm, but occurred at two different times. PCVII in the hearts and other tissues of affected piglets was confirmed by immunohistochemistry and virus isolation. Failure to detect porcine circoviruses in cases of reproductive failure prior to 1999 in areas of endemic infections supports the view that reproductive disease is a new clinical manifestation of PCVII infection, and further suggests that sexual, as well as vertical, modes of transmission are responsible for viral dissemination in the pig population.

Accordingly, inoculation of pigs, e.g., female pigs, such as sows or gilts, with a composition including at least one PCVII immunogen (e.g. from at least one strain chosen among strains Imp1008, Imp1010, Imp999, Imp1011-48285, Imp1011-48121, 1103 and 1121) (which composition can also include at least one immunogen from at least one other porcine pathogen such as at least one porcine parvovirus, wherein when a vector is used the vector can co-express both the PCVII immunogen(s) and the at least one immunogen of the at least one other porcine pathogen, e.g., a Helicobacter species or a PPV immunogen(s), inter alia), in a schedule of immunization as described above, can prevent myocarditis and/or abortion and/or intrauterine infection associated with PCVII, as well as post-weaning multisystemic wasting syndrome and other pathologic sequelae associated with PCVII.

Thus, the invention encompasses methods and compositions using PCVII immunogen for preventing myocarditis and/or abortion and/or intrauterine infection associated with porcine circovirus-2, as well as post-weaning multisystemic wasting syndrome and other pathologic sequelae associated with PCVII as well as against other infections such as Helicobacter species. Immunogen from strain 1103 and/or strain 1121 may be useful for methods and compositions using PCVII immunogen for preventing myocarditis and/or abortion and/or intrauterine infection associated with porcine circovirus-2 and may be combined with one or more immunogens derived from a Helicobacter species, advantageously H. cerdo according to the invention.

The PCVII immunogen can be any PCVII immunogen including any PCVII-expressing vector and the compositions comprising the PCVII immunogen and employed in the practice of this invention can be as in any herein cited document (or any document cited in herein cited documents) including any or all of: U.S. application Ser. No. 09/347,594, filed Jul. 1, 1999; French application No. 98 08777, filed Jul. 6, 1998; U.S. application Ser. No. 09/161,092, filed Sep. 25, 1998; U.S. application Ser. No. 09/082,558, filed May 21, 1998; French applications Nos. 97 12382, 98 00873 and 98 03707, filed Oct. 3, 1997, Jan. 22, 1998 and Mar. 20, 1998, respectively; WO-A-99 18214; the U.S. applications of Audonnet et al. and Bublot et al., Ser. Nos. 60/138,352 and 60/138,478, respectively, both filed Jun. 10, 1999, and Ser. Nos. 09/586,535 and 09/583,545, filed May 31 and Jun. 1, 2000, respectively (“DNA VACCINE-PCV”, and “PORCINE CIRCOVIRUS RECOMBINANT POXVIRUS VACCINE”, respectively); and WO99/29717 (all of which and documents cited therein and in the prosecution thereof being hereby incorporated herein by reference). Thus, the composition comprising the PCVII immunogen including the vector expressing PCVII immunogen can be prepared as in herein cited documents.

The at least one immunogen from at least one other porcine pathogen can be as described in any of the aforementioned or herein cited patent or literature publications (or documents cited therein), or as used in known porcine vaccines or immunogenic compositions, or as in WO 98/03658, published Jan. 29, 1998 from PCT/FR97/01313, filed Jul. 15, 1997; or French application 96 09338, filed Jul. 19, 1996; or U.S. application Ser. No. 09/232,468, filed Jan. 15, 1999 for (“POLYNUCLEOTIDE VACCINE FORMULA AGAINST PORCINE REPRODUCTIVE AND RESPIRATORY PATHOLOGIES”).

The amount of PCVII immunogen in compositions employed in the invention can be as described in any of the aforementioned or herein cited patent or literature publications (or documents cited therein). And, the amount of at least one immunogen from at least one other porcine pathogen can be as described in any of the aforementioned or herein patent or literature publications (or documents cited therein), or as used in known porcine vaccines or immunogenic compositions.

Compositions for use in the invention can be prepared in accordance with standard techniques well known to those skilled in the veterinary or pharmaceutical or arts. Such compositions can be administered in dosages and by techniques well known to those skilled in the veterinary arts taking into consideration such factors as the age, sex, weight, condition and particular treatment of the pig, and the route of administration. The compositions can be administered alone, or can be co-administered or sequentially administered with other compositions of the invention (e.g., other compositions comprising a PCVII immunogen) or with other prophylactic or therapeutic compositions (e.g., other porcine immunogenic or vaccine compositions). Thus, the invention also provides multivalent or “cocktail” or combination compositions and methods employing them. In this regard, reference is made to U.S. Pat. No. 5,843,456, incorporated herein by reference, and directed to rabies compositions and combination compositions and uses thereof.

Compositions of the invention may be used for parenteral or mucosal administration, preferably by intradermal or intramuscular routes. In particular for intradermal route, injection can be done using a needleless injector. When mucosal administration is used, it is possible to use oral, nasal, or ocular routes.

In such compositions the immunogen(s) may be in a mixture with a suitable carrier, diluent, or excipient such as sterile water, physiological saline, glucose or the like, and/or preferably with an adjuvant. The compositions can also be lyophilized or frozen. The compositions can contain auxiliary substances such as pH buffering agents, adjuvants, preservatives, polymer excipients used for mucosal routes, and the like, depending upon the route of administration and the preparation desired.

It is possible to use the SPT emulsion described on page 147 of “Vaccine Design, The Subunit and Adjuvant Approach” edited by M. Powell and M. Newman, Plenum Press, 1995, and the emulsion MF59 described on page 183 of this same book. For example the adjuvant-containing vaccine is prepared in the following way: 67% v/v of aqueous phase comprising the immunogen are emulsified in 2,3% w/v of anhydromannitol oleate, 2,6% w/v of oleic acid ethoxylated with 11 EO (ethylene oxide) and 28,1% v/v of light liquid paraffin oil (European Pharmacopea type) with the aid of an emulsifying turbomixer.

An alternative method for preparing the emulsion consists in emulsifying, by passages through a high-pressure homogenizer, a mixture of 5% w/v squalane, 2.5% w/v Pluronic® L121, 0.2% w/v of an ester of oleic acid and of anhydrosorbitol ethoxylated with 20 EO, 92.3% v/v of the aqueous phase comprising the immunogen.

A further instance of an adjuvant is a compound chosen from the polymers of acrylic or methacrylic acid and the copolymers of maleic anhydride and alkenyl derivative. Advantageous adjuvant compounds are the polymers of acrylic or methacrylic acid which are cross-linked, especially with polyalkenyl ethers of sugars or polyalcohols. These compounds are known by the term carbomer (Phameuropa Vol. 8, No. 2, June 1996). Persons skilled in the art can also refer to U.S. Pat. No. 2,909,462 (incorporated herein by reference) which describes such acrylic polymers cross-linked with a polyhydroxylated compound having at least 3 hydroxyl groups, preferably not more than 8, the hydrogen atoms of at least three hydroxyls being replaced by unsaturated aliphatic radicals having at least 2 carbon atoms. The preferred radicals are those containing from 2 to 4 carbon atoms, e.g. vinyls, allyls and other ethylenically unsaturated groups. The unsaturated radicals may themselves contain other substituents, such as methyl. The products sold under the name Carbopol® (BF Goodrich, Ohio, USA) are particularly appropriate. They are cross-linked with an allyl sucrose or with allyl pentaerythritol. Among then, there may be mentioned Carbopol® 974P, 934P and 971P. Among the copolymers of maleic anhydride and alkenyl derivative, the copolymers EMA® (Monsanto) which are copolymers of maleic anhydride and ethylene, linear or cross-linked, for example cross-linked with divinyl ether, are preferred. Reference may be made to J. Fields et al., Nature, 186: 778-780, 4 Jun. 1960, incorporated herein by reference.

From the point of view of their structure, the polymers of acrylic or methacrylic acid and the copolymers EMA® are preferably formed of basic units of the following formula: 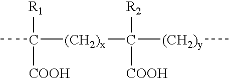
in which: R₁ and R₂, which are identical or different, represent H or CH₃; x=0 or 1, preferably x=1; and y=1 or 2, with x+y=2. For the copolymers EMA®, x=0 and y=2. For the carbomers, x=y=1.

The dissolution of these polymers in water leads to an acid solution that will be neutralized, preferably to physiological pH, in order to give the adjuvant solution into which the immunogenic, immunological or vaccine composition itself will be incorporated. The carboxyl groups of the polymer are then partly in COO⁻ form.

Preferably, a solution of adjuvant according to the invention, especially of carbomer, is prepared in distilled water, preferably in the presence of sodium chloride, the solution obtained being at acidic pH. This stock solution is diluted by adding it to the desired quantity (for obtaining the desired final concentration), or a substantial part thereof, of water charged with NaCl, preferably physiological saline (NaCL 9 g/l) all at once in several portions with concomitant or subsequent neutralization (pH 7.3 to 7.4), preferably with NaOH. This solution at physiological pH will be used as it is for mixing with the vaccine, which may be especially stored in freeze-dried, liquid or frozen form.

The polymer concentration in the final vaccine composition can be 0.01% to 2% w/v, e.g., 0.06 to 1% w/v, such as 0.1 to 0.6% w/v.

From this disclosure and the knowledge in the art, the skilled artisan can select a suitable adjuvant, if desired, and the amount thereof to employ in an immunological, immunogenic or vaccine composition according to the invention, without undue experimentation.

The immunogenic or vaccine compositions according to the invention may be associated to at least one live attenuated, inactivated, or sub-unit vaccine, or recombinant vaccine (e.g. poxvirus as vector or DNA plasmid) expressing at least one immunogen or epitope of interest from at least one another pig pathogen.

Compositions in forms for various administration routes are envisioned by the invention. And again, the effective dosage and route of administration are determined by known factors, such as age, sex, weight and other screening procedures which are known and do not require undue experimentation. Dosages of each active agent can be as in herein cited documents and/or can range from one or a few to a few hundred or thousand micrograms, e.g., 1 μg to 1 mg, for a subunit immunogenic, or vaccine composition; and, 10⁴ to 10¹⁰ TCID₅₀ advantageously 10⁶ to 10⁸ TCID₅₀ for an inactivated (titre before inactivation) immunogenic, or vaccine composition. For a live attenuated immunogenic or vaccine composition, the dose can be between 10¹ and 10⁸ TCID₅₀ advantageously 10³ and 10⁶ TCID₅₀.

Recombinants or vectors can be administered in a suitable amount to obtain in vivo expression corresponding to the dosages described herein and/or in herein cited documents. For instance, suitable ranges for viral suspensions can be determined empiracally. The viral vector or recombinant in the invention can be administered to a pig or infected or transfected into cells in an amount of about at least 10³ pfu; more preferably about 10⁴ pfu to about 10¹⁰ pfu, e.g., about 10⁵ pfu to about 10⁹ pfu, for instance about 10⁶ pfu to about 10⁸ pfu, per dose, e.g. of about 2 ml. And, if more than one gene product is expressed by more than one recombinant, each recombinant can be administered in these amounts; or, each recombinant can be administered such that there is, in combination, a sum of recombinants comprising these amounts.

In plasmid compositions employed in the invention, dosages can be as described in documents cited herein or as described herein. For instance, suitable quantities of each plasmid DNA in plasmid compositions can be 1 μg to 2 mg, preferably 50 μg to 1 mg. Documents cited herein regarding DNA plasmid vectors may be consulted by the skilled artisan to ascertain other suitable dosages for DNA plasmid vector compositions of the invention, without undue experimentation.

However, the dosage of the composition(s), concentration of components therein and timing of administering the composition(s), which elicit a suitable immunologenic response, can be determined by methods such as by antibody titrations of sera, e.g., by ELISA and/or seroneutralization assay analysis and/or by vaccination challenge evaluation in pig. Such determinations do not require undue experimentation from the knowledge of the skilled artisan, this disclosure and the documents cited herein. And, the time for sequential administrations can be likewise ascertained with methods ascertainable from this disclosure, and the knowledge in the art, without undue experimentation.

The PCVII immunogen can be obtained from PCVII or can be obtained from in vitro recombinant expression of PCVII gene(s) or portions or epitopes thereof. The Helicobacter immunogens can be obtained from Helicobacter or can be obtained from in vitro recombinant expression of Helicobacter gene(s) or portions or epitopes thereof. Methods for making and/or using vectors (or recombinants) for expression can be by or analogous to the methods disclosed in: U.S. Pat. Nos. 4,603,112, 4,769,330, 5,174,993, 5,505,941, 5,338,683, 5,494,807, 4,722,848, 5,942,235, 5,364,773, 5,762,938, 5,770,212, 5,942,235, 5,756,103, 5,766,599, 6,004,777, 5,990,091, 6,033,904, 5,869,312, 5,382,425, PCT publications WO 94/16716, WO 96/39491, WO 95/30018, Paoletti, Proc. Natl. Acad. Sci. USA (1996) 93:11341-11348, Smith et al., U.S. Pat. No. 4,745,051 (recombinant baculovirus), Richardson, C. D. (Editor), Methods in Molecular Biology 39, “Baculovirus Expression Protocols” (1995 Humana Press Inc.), Smith et al., (1983) Mol. Cell. Biol., 3: 2156-2165; Pennock et al., (1984), Mol. Cell. Biol. 4: 399-406; EPA 0 370 573, U.S. application Serial No. 920,197, filed Oct. 16, 1986, EP Patent publication No. 265785, U.S. Pat. No. 4,769,331 (recombinant herpesvirus), Roizman, (1996) Proc. Natl. Acad. Sci. USA 93:11307-11312; Andreansky et al., (1996) Proc. Natl. Acad. Sci. USA 93:11313-11318; Robertson et al. Proc. Natl. Acad. Sci. USA USA 93:11334-11340, 1996; Frolov et al., Proc. Natl. Acad. Sci. USA 93:11371-11377, October 1996, Kitson et al., J. Virol. 65, 3068-3075, 1991; U.S. Pat. Nos. 5,591,439, 5,552,143, WO 98/00166, allowed U.S. application Ser. Nos. 08/675,556, and 08/675,566 both filed Jul. 3, 1996 (recombinant adenovirus), Grunhaus et al., 1992, “Adenovirus as cloning vectors,” Seminars in Virology (Vol. 3) p. 237-52, 1993, Ballay et al. EMBO Journal, vol. 4, p. 3861-65, Graham, Tibtech 8, 85-87, April, 1990, Prevec et al., J. Gen Virol. 70, 429-434, PCT WO91/11525, Felgner et al. (1994), J. Biol. Chem. 269, 2550-2561, Science, 259:1745-49, 1993 and McClements et al., Proc. Natl. Acad. Sci. USA 93:11414-11420, 1996, and U.S. Pat. Nos. 5,591,639, 5,589,466, and 5,580,859 relating to DNA expression vectors, inter alia. See also WO 98/33510; Ju et al., Diabetologia, 41:736-739, 1998 (lentiviral expression system); Sanford et al., U.S. Pat. No. 4,945,050; Fischbach et al. (Intracel), WO 90/01543; Robinson et al., seminars in IMMUNOLOGY, vol. 9, pp. 271-283 (1997) (DNA vector systems); Szoka et al., U.S. Pat. No. 4,394,448 (method of inserting DNA into living cells); McCormick et al., U.S. Pat. No. 5,677,178 (use of cytopathic viruses); and U.S. Pat. No. 5,928,913 (vectors for gene delivery), as well as other documents cited herein. A viral vector, for instance, selected from pig herpes viruses, such as Aujeszky's disease virus, porcine adenovirus, poxviruses, especially vaccinia virus, avipox virus, canarypox virus, and swinepox virus, as well as DNA vectors (DNA plasmids) are advantageously employed in the practice of the invention.

The expression product from the PCVII gene(s) or portions thereof can be useful for generating antibodies such as monoclonal or polyclonal antibodies that are useful for diagnostic purposes. Similarly, expression product(s) from the PCVII gene(s) or portions thereof can be useful in diagnostic applications.

Further, one skilled in the art can determine an epitope of interest in a PCVII or Helicobacter immunogen, or in an immunogen of another porcine pathogen, without undue experimentation, from the disclosure herein and the knowledge in the art; see, e.g., WO 98/40500, incorporated herein by reference, regarding general information for determining an epitope of interest or an epitopic region of a protein, inter alia.

With particular reference to U.S. application Ser. No. 09/161,092, filed Sep. 25, 1998, U.S. application Ser. No. 09/082,558, filed May 21, 1998, French applications Nos. 97 12382, 98 00873 and 98 03707, filed Oct. 3, 1997, Jan. 22, 1998 and Mar. 20, 1998, respectively, and, WO-A-99 18214 (all incorporated herein by reference), particularly advantageous immunogenic, immunological or vaccine compositions are: An immunogenic or vaccine composition, collected from a cell culture in vitro which has been infected with a purified preparation of PCVII, such as a purified preparation of porcine circovirus selected from the group consisting of the preparations deposited at the ECACC, under the following references: accession No. V97100219 (strain Imp.1008), No. V97100218 (strain Imp.1010) and accession No. V97100217 (strain Imp.999) deposited Oct. 2, 1997, accession No. V98011608 (strain Imp.1011-48285) and No. V98011609 (strain Imp.1011-48121) deposited Jan. 16, 1998, accession No. 00012710 (strain 1103) and No. 00012709 (strain 1121) deposited Feb. 2, 2000, or an immunogenic or vaccine composition comprised of porcine circovirus produced on, and isolated from cell culture in vitro, these cells having been infected with a porcine circovirus capable of being isolated from a physiological sample or from a tissue sample, especially lesions, from a pig having the PMWS syndrome, e.g., such a composition wherein the porcine circovirus is produced on, and isolated from a pig kidney cell line, for instance, produced on, and isolated from PK/15 cells free from contamination with PCV-1; or such a composition comprising or prepared from a culture extract or supernatant, collected from a cell culture in vitro which have been infected with a such a circovirus. Thus, porcine circovirus can be an immunogen. For instance, the vaccine or immunogenic composition can comprise the attenuated live whole immunogen (e.g., virus), advantageously, in a veterinarily or pharmaceutically acceptable vehicle or diluent and optionally a veterinarily or pharmaceutically acceptable adjuvant, as well as, optionally, a freeze-drying stabilizer. The immunogen (e.g., virus) can be inactivated and the vaccine or immunogenic composition can additional and/or optionally comprise, a veterinarily or pharmaceutically acceptable vehicle or diluent and optionally a veterinarily or pharmaceutically acceptable adjuvant. The vaccine or immunogenic composition can comprise PCVII immunogens and/or immunogens of several porcine circoviruses (including PCVII or several strains of PCVII, and including PCV-1), as well as optionally additionally immunogens from another pig pathogen; e.g, PRRS, Mycoplasma hyopneumoniae, Actinobacillus pleuropneumoniae, E. coli, Pseudorabies, Hog cholera, Bordetella bronchiseptica, Pasteurella multocida, Swine Influenza, PPV (see also U.S. application Ser. No. 09/347,594, filed Jul. 1, 1999 and French application No. 98 08777, filed Jul. 6, 1998).

For the production of circovirus antigenic preparations, the circoviruses may be obtained after passage on cells, in particular cell lines, e.g. PK15 cells. The culture supernatants or extracts, optionally purified by standard techniques, may be used.

In the context of attenuated PCV, the attenuation may be carried out according to the customary methods, e.g. by passage on cells, preferably by passage on pig cells, especially cell lines, such as PK15 cells (for example from 20 to 150, especially of the order of 40 to 100, passages).

In the context of inactivated vaccine, the PCV, with the fractions that may be present, is inactivated according to techniques known to persons skilled in the art. The inactivation will be preferably carried out by the chemical route, e.g. by exposing the antigen to a chemical agent such as formaldehyde (formalin), paraformaldehyde, β-propiolactone or ethyleneimine or its derivatives, and/or by physical treatment. The preferred method of inactivation will be herein the exposure to a chemical agent and in particular to ethyleneimine or to β-propiolactone.

The immunogen in the vaccine or immunogenic composition can be expressed from a DNA fragment containing a sequence or fragment thereof (advantageously encoding at least one epitope) selected from the group consisting of the sequences designated by the references SEQ ID NOS: 1, 11, 12, and 24-30 (in U.S. application Ser. No. 09/161,092, filed Sep. 25, 1998, U.S. application Ser. No. 09/082,558, filed May 21, 1998, French applications Nos. 97 12382, 98 00873 and 98 03707, filed Oct. 3, 1997, Jan. 22, 1998 and Mar. 20, 1998, respectively, and, WO-A-99 18214). The immunogen in the vaccine or immunogenic composition can be expressed from a DNA fragment containing an ORF selected from the group consisting of ORFs 1 to 13, such as ORFs 4, 7, 10 and 13; preferably ORFs 4 and/or 13, of a PCVII strain, in particular of any one of the above identified strains (as designated in U.S. application Ser. No. 09/161,092, filed Sep. 25, 1998, U.S. application Ser. No. 09/082,558, filed May 21, 1998, French applications Nos. 97 12382, 98 00873 and 98 03707, filed Oct. 3, 1997, Jan. 22, 1998 and Mar. 20, 1998, respectively, and, WO-A-99 18214). Thus, the immunogen or a portion thereof, such as an epitope of interest can be obtained by in vitro expression thereof from a recombinant or a vector. The immunogen may be further purified and/or concentrated by the conventional methods.

The immunogen in the vaccine or immunogenic composition can be expressed in vivo by an expression vector comprising a DNA fragment containing a sequence or fragment thereof (advantageously encoding at least one epitope) selected from the group consisting of the sequences designated by the references SEQ ID NOS: 1, 11, 12, and 24-30. Similarly, the immunogen in the vaccine, immunogenic or immunological composition can be expressed in vivo by an expression vector comprising a DNA fragment containing an ORF selected from the group consisting of ORFs 1 to 13, such as ORFs 4, 7, 10 and 13; preferably ORFs 4 and/or 13, of a PCVII strain, in particular of any one of the above identified strains (as designated in U.S. application Ser. No. 09/161,092, filed Sep. 25, 1998, U.S. application Ser. No. 09/082,558, filed May 21, 1998, French applications Nos. 97 12382, 98 00873 and 98 03707, filed Oct. 3, 1997, Jan. 22, 1998 and Mar. 20, 1998, respectively, and, WO-A-99 18214). That is, the vaccine or immunogenic composition can comprise and expression vector that expresses the immunogen or a portion thereof, e.g., an epitope of interest, in vivo.

The expression vector can be any suitable vector such as a vector selected from DNA plasmids, bacteria such as E. coli, viruses such as baculovirus, herpesvirus such as Aujeszky's disease virus, adenovirus including porcine adenovirus, poxviruses, especially vaccinia virus, avipox virus, canarypox virus, and swinepox virus, inter alia (See also the U.S. applications of Audonnet et al. and Bublot et al., Ser. Nos. 60/138,352 and 60/138,478, respectively, both filed Jun. 10, 1999 (“DNA VACCINE-PCV”, and “PORCINE CIRCOVIRUS RECOMBINANT POXVIRUS VACCINE”, respectively).

Accordingly, the invention also comprehends nucleic acid molecules and vectors containing them, as well as expression products therefrom, compositions comprising such nucleic acid molecules and/or vectors and/or expression products, as well as methods for making and using any or all of these embodiments. The invention especially encompasses herein disclosed nucleic acid molecules, nucleic acid molecules of documents cited or referenced herein, including PCT WO 99/29717, fragments thereof, e.g., ORFs and/or fragments encoding an immunogen or epitope, as well as nucleic acid molecules of strains 1103 and/or 1121, and fragments thereof, as well as vectors comprising these nucleic acid molecules, compositions comprising these nucleic molecules, vectors, or expression products therefrom, compositions comprising such expression products, primers or probes for such nucleic acid molecules, and uses or methods involving these embodiments, e.g., for detecting, diagnosing, assaying for PCVII, for inducing an immunologenic or protective response, and the like. Indeed, this invention encompasses any inventions disclosed and/or claimed in PCT WO 99/29717 or any National application claiming priority therefrom or from the U.S. Provisionals from which that PCT claims priority.

As earlier mentioned, embodiments of the invention can include antibodies. Such antibodies can be polyclonal or monoclonal antibodies; for instance, prepared from the aforementioned circovirus, or from a polypeptide encoded by a DNA fragment having a sequence selected from the group consisting of SEQ ID NOS: 1, 11, 12, and 24-30 or from a polypeptide from expression by a vector comprising a sequence selected from the group consisting of SEQ ID NOS: 1, 11, 12, and 24-30; or from a polypeptide from expression by a vector comprising DNA including an ORF selected from the group consisting of ORFs 1 to 13 (as designated in U.S. application Ser. No. 09/161,092, filed Sep. 25, 1998, U.S. application Ser. No. 09/082,558, filed May 21, 1998, French applications Nos. 97 12382, 98 00873 and 98 03707, filed Oct. 3, 1997, Jan. 22, 1998 and Mar. 20, 1998, respectively, and, WO-A-99 18214). The skilled artisan may use techniques known in the art to elicit antibodies and to generate monoclonal or polyclonal antibodies. Antibodies and antigens can be used in diagnostics.

U.S. application Ser. No. 09/161,092, filed Sep. 25, 1998, U.S. application Ser. No. 09/082,558, filed May 21, 1998, French applications Nos. 97 12382, 98 00873 and 98 03707, filed Oct. 3, 1997, Jan. 22, 1998 and Mar. 20, 1998, respectively, and, WO-A-99 18214 also provide for probes or primers which can be useful, for instance, in detecting PCVII DNA, as well as for amplifying PCVII DNA, e.g., for preparing an expression vector. A probe or primer can be any stretch of at least 8, preferably at least 10, more preferably at least 12, 13, 14, or 15, such as at least 20, e.g., at least 23 or 25, for instance at least 27 or 30 nucleotides in PCVII genome or a PCVII gene which are unique to PCVII or which are in PCVII and are least conserved among the PCV or circovirus family. As to PCR or hybridization primers or probes and optimal lengths therefor, reference is also made to Kajimura et al., (1990) GATA 7: 71-79. Hybridization is advantageously under conditions of high stringency, as the term “high stringency” would be understood by those with skill in the art (see, for example, Sambrook et al., 1989, Molecular Cloning: A Laboratory Manual, Cold Spring Harbor Laboratory Press, Cold Spring Harbor, N.Y. and Hames and Higgins, eds., 1985, Nucleic Acid Hybridization, IRL Press, Oxford, U.K.). Hybridization will be understood to be accomplished using well-established techniques, including but not limited to Southern blot hybridization, Northern blot hybridization, in situ hybridization and, advantageously, Southern hybridization to PCR-amplified DNA fragments.

Like probes or primers, peptides which are not full-length PCVII proteins are part of invention and can be any stretch of at least 8, preferably at least 10, more preferably at least 12, 13, 14, or 15, such as at least 20, e.g., at least 23 or 25, for instance at least 27 or 30 amino acids in PCVII which are unique to PCVII or which are in PCVII and are least conserved among the PCV and/or circovirus family. Alternatively or additionally, the amino acids of the invention that are not full-length PCVII proteins can be an epitopic region of a PCVII protein.

And, as to DNA and protein sequences used in the invention, they can have homology, identity or similarity and degrees thereof as defined in U.S. application Ser. No. 09/347,594, filed Jul. 1, 1999 with homology, identity or similarity advantageously determined as discussed in U.S. Ser. No. 09/347,594.

The invention further relates to novel bacterial strains isolated from the stomachs of infected pigs. The bacterial species are of the genus Helicobacter, including the novel species H. cerdo that is distinguished, for example, from known species on the basis of SDS-PAGE analysis of the proteins of the isolates.

The subject of the present invention further relates to a vaccination of pigs using a porcine circovirus, in particular type I or type II, advantageously type II, vaccine, combined with a vaccination with a porcine Helicobacter vaccine. This is understood to mean vaccination with either a bivalent vaccine, or the simultaneous use in pigs of a porcine circovirus vaccine and of a porcine Helicobacter vaccine. An advantageous Helicobacter strain is Helicobacter cerdo.

The subject of the present invention is also an antigenic preparation directed against the gastro-esophageal ulceration and PMWS syndrome and which may comprise at least one porcine circovirus antigen (preferably type II circovirus) and at least one Helicobacter antigen. In accordance with the invention, the porcine circovirus antigen (preferably type II circovirus) and the Helicobacter antigen comprise, independently of each other, an antigen chosen from the group consisting of an attenuated live whole antigen, an inactivated whole antigen, a subunit antigen, a recombinant live vector and a DNA vector. It is understood that the combination according to the invention may involve the use of any appropriate antigen or antigenic preparation form, it being understood that it is not necessary to use the same form for a given combination. The antigenic preparation may comprise, in addition, as is known per se, a vehicle or excipient acceptable from the veterinary point of view, and optionally an adjuvant acceptable from the veterinary point of view.

The subject of the present invention is also immunogenic compositions or vaccines against the gastro-esophageal ulceration and PMWS syndrome, comprising effective quantities of a porcine circovirus and a Helicobacter antigenic preparation as described above, in a vehicle or excipient acceptable from the veterinary point of view, and optionally an adjuvant acceptable from the veterinary point of view. An immunogenic composition elicits an immunological response that can, but need not be, protective. A vaccine composition elicits a protective response. Accordingly, the term “immunogenic composition” includes a vaccine composition (as the former term can be protective composition).

The subject of the invention is also an immunological or a vaccination kit containing, packaged separately, an antigenic preparation or an immunogenic composition or a vaccine against the porcine circovirus and an antigenic preparation or an immunogenic composition or a vaccine against the porcine Helicobacter. This kit may have the various characteristics set out above for the antigenic preparations, immunogenic compositions and vaccines.

The subject of the invention is also a method of immunization or of vaccination against the gastro-esophageal ulceration and PMWS syndrome comprising the administration of an immunogenic composition or a vaccine against the porcine circovirus and of an immunogenic composition or a vaccine against the Helicobacter or the administration of a bivalent immunogenic composition or vaccine, comprising, in the same formulation, an antigenic preparation specific to a virus and bacteria. This method of immunization or vaccination uses in particular the vaccines as defined above.

The subject of the invention is also the use of an antigenic preparation or of an immunogenic composition or a vaccine against the Helicobacter, as in particular defined supra, for the preparation of a pharmaceutical composition intended to be used in the context of the prevention of the gastro-esophageal ulceration syndrome, in combination with an antigenic preparation or an immunogenic composition or a vaccine against the porcine circovirus.

For the production of circovirus antigenic preparations, the circoviruses may be obtained after passage on cells, in particular cell lines, e.g. PK15 cells. The culture supernatants or extracts, optionally purified by standard techniques, may be used as antigenic preparation.

In the context of attenuated antigenic preparations and attenuated immunogenic compositions or vaccines, the attenuation may be carried out according to the customary methods, e.g. by passage on cells, preferably by passage on pig cells, especially cell lines, such as PK15 cells (for example from 50 to 150, especially of the order of 100, passages). These immunogenic compositions and vaccines comprise in general a vehicle or diluent acceptable from the veterinary point of view, optionally an adjuvant acceptable from the veterinary point of view, as well as optionally a freeze-drying stabilizer.

These antigenic preparations, immunogenic compositions and vaccines will preferably comprise from 10³ to 10⁷ TCID50 of the attenuated virus or bacteria in question.

They may be antigenic preparations, immunogenic compositions and vaccines based on inactivated whole antigen. The inactivated immunogenic compositions and vaccines comprise, in addition, a vehicle or a diluent acceptable from the veterinary point of view, with optionally in addition an adjuvant acceptable from the veterinary point of view.

The circoviruses or the Helicobacter isolates according to the invention, with the fractions that may be present, are inactivated according to techniques known to persons skilled in the art. The inactivation will be preferably carried out by the chemical route, e.g. by exposing the antigen to a chemical agent such as formaldehyde (formalin), paraformaldehyde, β-propiolactone or ethyleneimine or its derivatives. An advantageous method of inactivation will be herein the exposure to a chemical agent.

Advantageously, the inactivated antigenic preparations and the inactivated immunogenic compositions and vaccines according to the invention will be supplemented with adjuvant, advantageously by being provided in the form of emulsions, for example water-in-oil or oil-in-water, according to techniques well known to persons skilled in the art. It will be possible for the adjuvant character to also come from the incorporation of a customary adjuvant compound into the active ingredient.

Among the adjuvants that may be used in the combination vaccines of the invention, there may be mentioned by way of example aluminium hydroxide, the saponines (e.g. Quillaja saponin or Quil A; see Vaccine Design, The Subunit and Adjuvant Approach, 1995, edited by Michael F. Powel and Mark J. Newman, Plennum Press, New-York and London, p. 210), Avridine.RTM. (Vaccine Design p. 148), DDA (Dimethyldioctadecyl-ammonium bromide, Vaccine Design p. 157), Polyphosphazene (Vaccine Design p. 204), or alternatively oil-in-water emulsions based on mineral oil, squalene (e.g. SPT emulsion, Vaccine Design p. 147), squalene (e.g. MF59, Vaccine Design p. 183), or water-in-oil emulsions based on metabolizable oil (preferably according to WO-A-94 20071) as well as the emulsions described in U.S. Pat. No. 5,422,109. It is also possible to choose combinations of adjuvants, for example Avridine.RTM. or DDA combined with an emulsion.

The adjuvants for live vaccines described above can be selected from those given for the inactivated. The emulsions are preferred. To those indicated for the inactivated vaccine, there may be added those described in WO-A-9416681. As freeze-drying stabilizer, there may be mentioned by way of example SPGA (Bovarnik et al., J. Bact. 59, 509, 950), carbohydrates such as sorbitol, mannitol, starch, sucrose, dextran or glucose, proteins such as albumin or casein, derivatives of these compounds, or buffers such as alkali metal phosphates.

The antigenic preparations, immunogenic compositions and vaccines according to the invention may comprise one or more active ingredients (antigens) of one or more circoviruses and/or Helicobcater spp. according to the invention.

In the context of the combined immunization or vaccination programmes of the invention, it is also possible to combine the immunization or vaccination against the porcine circovirus and the porcine Helicobacter with an immunization or vaccination against other pig pathogens, in particular those that could be associated with the PMWS syndrome. The immunogenic composition or vaccine according to the invention may therefore comprise another valency corresponding to another pig pathogen such as, but not limited to, PRRS (Porcine Reproductory and Respiratory Syndrome) and/or Mycoplasma hyopneumoniae, and/or E. coli, and/or Atrophic Rhinitis, and/or Pseudorabies (Aujeszky's disease) virus and/or porcine influenza and/or Actinobacillus pleuropneumoniae and/or Hog cholera, and combinations thereof. Preferably, the programme of immunization or vaccination and the vaccines according to the invention will combine immunizations or vaccinations against the circovirus and the parvovirus, and the PRRS (WO-A-93/07898, WO-A-94/18311, FR-A-2 709 966; C. Charreyre et al., Proceedings of the 15^{th} IPVS Congress, Birmingham, England, Jul. 5-9, 1998, p 139; and/or Mycoplasma hyopneumoniae (EP-A-597 852, EP-A-550 477, EP-A571 648; O. Martinon et al. p 157, 284, 285 and G. Reynaud et al., p 150, all in the above-referenced Proceedings of the 15.sup.th IPVS Congress) and/or porcine influenza. It is thus possible to use any appropriate form of immunogenic composition or vaccine, in particular any available commercial vaccine, so as to combine it with the immunogenic composition or vaccine against the porcine circovirus and porcine Helicobacter, as described herein.

The subject of the present invention is, therefore, also multivalent immunogenic compositions and vaccines, multivaccine kits, and combined immunization or vaccination methods which make it possible to use such combined immunization or vaccination programmes.

One aspect of the invention, therefore, is an immunogenic composition for eliciting an immunological response against a Helicobacter species and porcine circovirus comprising at least one Helicobacter antigen and at least one porcine circovirus antigen, and a veterinarily acceptable vehicle or excipient.

In one embodiment of the immunogenic compositions according to the invention, the porcine circovirus antigen may comprise at least one porcine circovirus type II antigen.

In other embodiments of the invention, the Helicobacter antigen is an antigen of Helicobacter cerdo, Helicobacter heilmanii, or Helicobacter pylori.

In the various embodiments of the invention, the porcine circovirus type II antigen is at least one antigen of a porcine circovirus type II deposited at the ECACC selected from group consisting of: porcine circovirus type II accession No. V97100219, porcine circovirus type II accession No. V97100218, porcine circovirus type II accession No. V97100217, porcine circovirus type II accession No. V98011608, and porcine circovirus type II accession No. V98011609.

In one embodiment of the invention, the porcine circovirus type II antigen is an attenuated virus porcine circovirus type II or an inactivated porcine circovirus type II.

Another embodiment of the invention further comprises a veterinarily acceptable adjuvant and, optionally, a freeze-drying stabilizer.

In the various embodiments of the invention, the Helicobacter antigen can be an antigen of Helicobacter cerdo.

In embodiments of the invention, the porcine circovirus type II antigen can comprise an antigen encoded by a porcine circovirus type II open reading frame (ORF) selected from the group consisting of ORFs 1, 2, 3, 4, 5, 6, 7, 8, 9, 10, 11, 12, and 13 as identified in PCVII strain 1010, or the equivalent ORFs of other PCVII strains.

In other embodiments of the invention, the porcine circovirus type II antigen comprises a vector that contains and expresses in vivo an antigen encoded by a porcine circovirus type II open reading frame (ORF) selected from the group consisting of ORFs 1, 2, 3, 4, 5, 6, 7, 8, 9, 10, 11, 12, and 13 as identified in PCVII strain 1010, or the equivalent ORFs of other PCVII strains.

In yet other embodiments of the invention, the vector is selected from the group consisting of a DNA plasmid, a linear DNA molecule, and a recombinant virus.

In embodiments of the invention, the recombinant virus may be selected from the group consisting of pig herpes virus, porcine adenovirus, and poxvirus.

In yet other embodiments of the invention, the recombinant virus is selected from the group consisting of Aujesky's disease virus, vaccinia virus, avipox virus, canarypox virus, and swine pox virus.

In the embodiments of the invention, the Helicobacter antigen may be selected from the group consisting of an attenuated Helicobacter strain, an inactivated Helicobacter strain, or a subunit of a Helicobacter strain, and the porcine circovirus antigen may be selected from the group consisting of an attenuated porcine circovirus, an inactivated porcine circovirus or a subunit of porcine circovirus, and a vector that contains and expresses in vivo a nucleic acid molecule encoding at lest one of the above antigens. The vector may be selected from, but is not limited to, the group consisting of a DNA plasmid, a linear DNA molecule, and a recombinant virus; and optionally an additional antigen of another porcine pathogen.

One embodiment of the invention further encompasses an additional antigen of another porcine pathogen selected from the group consisting of, but not limited to: an antigen of PRRS virus, an antigen of Mycoplasma hypopneumoniae, an antigen of Actinobacillus pleuropneumoniae, an antigen of E. coli, an antigen of Atrophic Rhinitis, an antigen of Pseudorabies virus, an antigen of Hog cholera, an antigen of Swine Influenza, and combinations thereof.

In another embodiment of the invention, the antigen of porcine circovirus comprises antigens of a plurality of porcine circoviruses.

Another aspect of the invention is a method for inducing an immunological response against a Helicobacter strain and a porcine circovirus comprising administering to a porcine an immunogenic composition having an immunogen derived from each microbial type.

Yet another aspect of the invention is a kit for preparing the immunogenic composition and which may encompass at least one Helicobacter antigen and at least one porcine circovirus antigen, wherein (i) and (ii) are packaged separately or together. In one embodiment of this aspect of the invention, the porcine circovirus antigen comprises at least one porcine circovirus type II antigen.

It should be understood that the present invention is not limited to the specific compositions or methods described herein and that any composition having a formula or method steps equivalent to those described falls within the scope of the present invention. Preparation routes of the composition and method steps are merely exemplary so as to enable one of ordinary skill in the art to make the composition and use it according to the described process and its equivalents. It will also be understood that although the form of the invention shown and described herein constitutes advantageous embodiments of the invention, it is not intended to illustrate all possible forms of the invention. The words are words of description rather than of limitation. Various changes and variations may be made to the present invention without departing from spirit and scope of the invention.

The invention is illustrated by the following non-limiting examples:

### EXAMPLES

### Example 1

### Methods for Isolation and Characterization of PCV Isolates

Cell Cultures: The Dulac cell line, a PCV-free PK15 derivative, was obtained from Dr. John Ellis (University of Saskatchewan, Saskatoon, Saskatchewan). The Vero cell line was obtained from American Type Culture Collection (ATCC), Manassas, Va. These cells were cultured in media as suggested by the ATCC and incubated at 37° C. with 5% CO₂.

Porcine Circoviruses: The classic PCVI was isolated from persistently infected PK15 cells (ATCC CCL33). Isolate PCVII 412 was obtained from lymph nodes of a piglet challenged with the lymph node homogenate from PMWS-affected piglets. This challenged piglet had been diagnosed with PMWS. Isolate PCVII 9741 was isolated from the buffy-coat of peripheral blood from a PMWS-affected piglet of the same herd after the isolation of PCVII 412. Isolate PCVII B9 was isolated from an affected piglet in a United States swine herd with a PMWS clinical outbreak in the fall of 1997.

Propagation of PCVI: PCVI from persistently infected PK15 cells was grown and purified using a modified method of Tischer et al (1987) Arch. Virol. 96:39-57. Briefly, PCV harvested from PK15 cells was used to super-infect a monolayer of PK15 cells at about 1 moi for two hours before the cells were treated with 300 mM D-glucosamine. After washing the cells once, DMEM (Gibco, catalog number 21013) with 5% FBS was added to the cells and the cells were incubated for an additional four days. The infected cells were scraped off and collected after centrifugation at 1500×g for 15 minutes. The cell pellet was then treated with 0.5% of Triton X-114 at 37° C. for 30 minutes. After another low speed centrifugation to remove cellular debris, an equal amount of Freon (Sigma catalog number T-5271) was added to the supernatant and the mixture was homogenized for one minute using a Polytron at maximum speed. The mixture was then centrifuged and the top layer collected and mixed with an equal volume of 0.1 M PBS. The virus pellet was collected after ultra centrifugation into a 20% sucrose cushion at 210,000×g for 30 minutes.

Culture of the Field Isolates (PCVII): The isolate PCVII 412 was cultured and purified in a similar manner as PCVI, except Dulac cells were used. The isolate PCVII B9 was grown in heterogenic Vero cells transfected with self-ligated full-length PCR products from the United States PMWS outbreak. Therefore, the possibility of contamination from other pig pathogens was eliminated. The B9-transfected Vero cells were continuously passed and treated with 300 mM D-glucosamine as described above.

Viral DNA Isolation: Viral DNA was extracted from variable sources, including pellets of infected Dulac and Vero cells, peripheral blood buffy-coat cells, tissues from infected animals and serum. The tissue samples were treated with proteinase K and viral DNA was extracted using either phenol/chloroform or Qiagen tissue kit (Qiagen, Santa Clarita, Calif.). DNA from peripheral blood buffy coat cells of heparinized blood and serum was similarly collected using the Qiagen blood kit.

Infection of Piglets: Piglets were derived from specific pathogen-free sows. At one day of age, each piglet received approximately one gram of lymph nodes collected from PMWS-affected piglets. The tissue homogenate was distributed equally between the oral and intraperitoneal routes. Ten piglets were used in each of the experimental groups and observed daily for 7 weeks. Two groups were challenged and 2 were uninfected controls. Two groups, one challenged and one control, were also treated with cyclosporin A (2 mg/kg) at Day 0 and Day 14. The piglets were fed canned milk (Carnation) and water (50:50) until they self-weaned to high nutrient density commercially prepared feed.

PCR, cloning and sequencing of the field PCV isolates: A two-step approach was used for the initial cloning of isolate PCVII 412 viral genomic DNA. A primer that hybridized to the conserved loop stem sequences, Loop⁻ (Table 1), was designed to perform a single-primed PCR taking advantage of the complementary sequences and the circular nature of PCV genomic DNA. The PCR reaction for the single-primed PCR was a two-stage process. The first stage consisted of 5 cycles of denaturing at 94° C. for 1 minute, annealing at 37° C. for 30 seconds and extension at 72° C. for 2 minutes. The second stage consisted of 25 cycles of a similar program except the annealing temperature was increased to 52° C. The PCR products were cloned into a TA cloning vector (Invitrogen, Carlsbad, Calif.). Both strands of three different clones were sequenced to ensure sequence fidelity. Based on the sequences obtained, primer 1000-and RIF were designed in the noncoding region of the viral DNA sequences and used to clone the full-length viral genome. The sequences of all the primers used in this study are shown in Table 1 below. The sequences of the loop region were then obtained from the full-length clone. Sequences of isolate PCVII 9741 and PCVII B9 were obtained from purified PCR products. Automated DNA sequencing performed by Plant Biotechnology Institute of NRC, Canada was used with several internal primers. The sequences of isolates PCVII 412 (AF085695; SEQ ID NO: 1), PCVII 9741 (AF086835; SEQ ID NO: 11) and PCVII B9 (AF086834 SEQ ID NO: 12) were deposited with the National Center for Biotechnology Information (NCBI).

TABLE 1
Sequences of Primers Used in the Studies

| | | |
|---|---|---|
| | | SEQ |
| Primer Name | Primer Sequence | ID NO: |

| | | |
|---|---|---|
| Loop⁻ | ACTACAGCAGCGCACTTC | 13 |
| 1000− | AAAAAAGACTCAGTAATTTATTTCATATGG | 14 |
| R1F | ATCACTTCGTAATGGTTTTTATT | 15 |
| 1710+ | TGCGGTAACGCCTCCTTG | 16 |
| 850− | CTACAGCTGGGACAGCAGTTG | 17 |
| 1100+ | CATACATGGTTACACGGATATTG | 18 |
| 1570− | CCGCACCTTCGGATATACTG | 19 |
| 1230− | TCCCGTTACTTCACACCCAA | 22 |
| 400+ | CCTGTCTACTGCTGTGAGTA | 23 |

Sequence Analyses: The sequences of other circoviruses were obtained from NCBI. Various public domains were used for the sequence analysis, such as Biology workbench, Blast search, DNA/protein analysis tools, etc. The sequence alignments were generated using Clustal W program and phylogenetic trees were created by PAUP 3.1 program (David L. Swofford, Laboratory of Molecular Systematics, MRC534, MRC at Smithsonian Institution, Washington, D.C.).

Multiplex PCR: Two sets of primers were designed to identify the PCV group-specific sequences and strain-specific sequences. The primer pair 1710+/850− is PCV-group specific and 1100+/1570− is the novel PCV strain-specific pair, which differentiates the novel PCV from the one derived from PK15 cells. The two sets of primers have similar annealing temperatures for the PCR reaction and were used together at 0.5 μM concentration in a standard hot start PCR reaction. Either Ampli Taq Gold (Perkin Elmer) or Plentinum Taq (Gibco) was used.

Antiserum: Rabbit anti-PCVII 412 pooled serum was obtained from two rabbits injected with purified isolate PCVII 412 at 50 μg/dose in an oil-in-water emulsion. The injection was repeated 3 times at 21-day intervals. Pig anti-PMWS serum was collected from convalescent pigs from PMWS affected herds.

ELISA: Purified PCV was diluted in sodium carbonate buffer (0.05 M) pH 9.6 to a concentration of 0.5 μg per 100 μL and used to coat Immulon II plates (Dynatech Laboratories, Inc.). The plates were washed six times with TTBS (20 mM Tris-HCl, 500 mM NaCl, 0.05% of Tween 20, pH 7.5) before serially diluted primary rabbit or pig antibody was added. After six washes with TTBS, alkaline phosphatase-conjugated secondary antibodies (1/5000 dilution), either anti-rabbit or anti-pig (Kirkegaard & Perry), were added. Plates were developed with 100 μL/well of p-Nitrophenyl Phosphate (PNPP, 3 g/L) in 1 M diethanolamine, 0.5 MgCl₂, pH 9.8 and the plates were read on an ELISA reader (BioRad) at 405/490 nm.

FACS Analysis of lymphocyte surface markers: Blood samples were collected from PMWS affected piglets in the field and negative control. The RBC was lysed and WBC was stained with anti-pig CD3, CD4 and CD8 monoclonal antibodies, and followed by fluorescence labeled anti-mouse secondary antibody. The specifically labeled cells were fixed with 2% formaldehyde and 5000 cells were counted using FACS system (Becton Dickinson).

### Example 2

### PMWS Reproduction

PMWS has not been reproduced under controlled conditions, nor have etiology studies been performed. In order to determine the causative agent of this disease, a number of tissues were collected from PMWS-affected pigs, as described above in Example 1, and studied. Lymph nodes displayed the most apparent gross lesions, histopathological changes and circovirus infection was confirmed by immunostaining. Accordingly, the lymph nodes were used in the challenge experiments described above.

The challenge experiments, conducted as described in Materials and Methods were successful in producing PMWS in pigs. In particular, some piglets died of the infection and asymptomatically infected piglets developed PMWS-like microscopic lesions by the end of the trial.

In another challenge experiment, the starting material used was lung tissue of pig with chronic wasting and lymph node enlargement. These clinical signs are characteristic of PMWS. The tissue was combined with sterile 0.1 M phosphate-buffered saline (PBS) and homogenized by passage through a polytron mixer. The crude tissue homogenate was used to challenge pigs. In particular, a total of 40 piglets (approximately 1 day of age) were randomly (balanced by litter of birth, gender and body weight) assigned to “tissue challenge,” “tissue challenge with Cyclosporin-A,” “control,” or “Cyclosporin-A” treatment groups. The cyclosporin treatment had no clinical or hematological effect on the treated pigs except that cyclosporin was detected in the blood of those pigs three hours after the drug was administered. Hence, groups were collapsed across cyclosporin treatment for analysis.

In general, postmortem signs of PMWS disease in the challenged pigs included enlarged lymph nodes and incomplete collapse of lung tissue. Postmortem signs of PMWS disease were detected in significantly (p<0.01; two-tailed Fishers exact-test) more pigs in the group treated with tissue extract (7 pigs out of 9) than in the group treated with placebo (2 pigs out of 18). The average daily gain in the group treated by injection of tissue extract (212 g/d) was not substantially different from the group given the placebo (202 g/d).

Blood samples were obtained throughout the experiment and tissue samples were taken postmortem. The samples were tested for PCVII viral DNA by PCR that resulted in an 830 base pair product. Four of the pigs given the lung tissue extract had positive blood samples; whereas none of the pigs given placebo had PCVII DNA detected in their blood. PCVII was detected in one or more tissues from 7 of the 8 surviving pigs in the “virus challenge” treatment group whereas all tissues from pigs in the control group were negative for PCVII. Contingency table analysis showed a significant difference (p<0.001; two-tailed Fishers exact-test).

In another challenge experiment, lung tissue of pig with chronic wasting and lymph node enlargement was collected and tissue debris removed by centrifugation (8000 rpm for 30 minutes). The supernatant was applied to a cesium chloride step-gradient and centrifuged at 100,00×g. Bands appeared between 41% CsCl₂ (1.28 gm/ml) and 63% (1.40 gm/ml). These bands were applied to a 30% CsCl₂ “foot” and centrifuged for 2 hours at 100,000×g. The pellet was resuspended in 15 mL of sterile 0.1 M PBS.

A total of 20 weaned piglets (approximately three weeks of age) were randomly (balanced by litter of birth, gender and body weight) assigned to “control” or “virus challenge” treatment groups. Pigs were weaned on Day 0 at approximately three weeks of age. In general, clinical signs of PMWS disease included enlarged lymph nodes and wasting or poor growth. Enlarged lymph nodes were detected in significantly (p<0.02; two-tailed Fisher exact-test) more pigs in the group treated with virus (7 pigs) than in the group treated with placebo (1 pig). The average daily gain in the group treated by virus injection (580 gm/d) tended to be less than the group given the placebo (616 gm/d), but the difference was not significant (p=0.17; two-tailed paired t-Test). There was no difference between groups in the relative mass of internal organs (liver, lung, heart, spleen, kidneys).

Blood samples that were obtained throughout the experiment and tissue samples that were taken postmortem were tested for PCVII viral DNA using the PCR techniques described above.

All blood samples including those taken just prior to euthanasia were negative for PCVII. PCVII was detected in one or more tissues for 8 of the 10 pigs in the “virus challenge” treatment group whereas all tested tissues from pigs in the control group were negative for PCVII. Contingency table analysis showed that this was a significant difference (p<0.001; two-tailed Fishers exact-test).

In conclusion, these experiments confirm that injection of weaned piglets with tissue extracts and gradient-purified viral material containing PCVII results in infection of multiple tissues. The infection persists for a duration of at least eight weeks.

### Example 3

### Isolation and Propagation of PCVII

To determine the presence of an infectious causative agent(s) for PMWS, various tissues from pig #412, an experimentally challenged piglet sacrificed 21 days post-infection, were used for viral isolation. After continued passage of lymph node samples from pig #412 in Dulac cells, virus accumulation or adaptation was observed. A unique pattern of cytopathic effect initially developed, followed by increasing virus titer, as determined by ELISA using the standard Berlin anti-PCV antibody, as described above.

The existence of circovirus in Dulac cells infected with isolate PCVII 412 was then detected by electron microscopic examination. After six passages, viral structure proteins could be detected consistently, using a western blot assay.

### Example 4

### Specific Anti-PCVII Antibodies in Asymptomatically Infected and Convalescent Piglets in PMWS-Affected Herds

Because it appeared that porcine circoviruses possessed some heterogeneity, ELISAs were performed using sera of piglets, collected from a herd with a PMWS outbreak, against the PCV and isolate PCVII 412 virus. Most of the asymptomatically PCVII-infected and convalescent piglets developed specific antibodies against PCVII, not PCVI.

### Example 5

### Isolation, Cloning and Sequencing of PCVII Virus and Viral Genomic DNA

In order to explore genetic differences between the two strains of porcine circoviruses, viral DNA was extracted from infected Dulac cells. Considering the possible genetic unrelatedness between PCVI and PCVII, the approach was to design primer(s) from the most conserved region. Previous analysis of the PK15 PCV DNA sequences (Mankertz et al. (1997) J. Gen. Virol. 71:2562-2566; Meehan et al. (1997) J. Gen. Virol. 78:221-227) revealed a stem loop structure in the origin of replication. A single primer, targeting the inverted repeat sequence of the stem loop region, Loop⁻ (see Example 1, Table 1), was designed because of the highly conserved nature of this important domain. The amplification of the PCVII 412 viral DNA by single primer PCR was successful. After cloning into a TA cloning vector, the viral genomic sequence was obtained by automated sequencing from several clones and both senses to ensure fidelity. The actual sequence of the stem loop or primer region was then obtained from a second full-length clone generated by primers of 1000- and RIF from the only non-coding region of the virus. The nucleotide sequence for PCV 412 (SEQ ID NO: 1) is shown in the top line of FIGS. 2A-2C.

Using similar primers, other PCVII isolates, including PCVII 9741 from the same herd as PCVII 412, and PCVII B9 from a PMWS outbreak in the United States, were obtained. These strains were sequenced and compared to PCVII 412 and PCVI. See FIGS. 2A-2C for a comparison of PCVII 412 with PCVI and FIGS. 4A-4B for comparisons of the PCVII 412 sequence (SEQ ID NO: 1) with the various PCV isolates.

The results of a phylogenetic analysis using the PAUP 3.1 program suggested that the new PMWS isolates were closely related and in a different cluster with PCVI. These isolates were therefore termed “PCVII” isolates. The percent nucleotide sequence homologies among isolates of the novel porcine circovirus were more than 99% identical. In contrast, comparison of these nucleotide sequences with the PK15 PCVI showed only 75.8% overall nucleotide sequence homology. Comparative analysis of nucleotide sequences in different regions further revealed that the putative replication-associated protein gene of these two viruses share 81.4% homology, while the nucleotide sequences of the other large ORF was only 67.6% homologous.

Furthermore, nucleotide insertions and deletions were found in three regions. There are 13 base insertions in the new isolates between PCVI sequence 38-61 that flank the start codon for the putative 35.8 kd protein encoded by ORF 1. The area of PCVI 915-1033, containing 15 base indels, was at the ends and the joint region of the two largest ORFs (the other ORF was antisense) of the porcine circoviruses. The third region, covering PCVI sequence from 1529-1735 with 15 base indels, locates at the amino end of a putative 27.8 kd protein encoded by ORF 6. PCVI sequences were also compared with the available sequences of the rest of the members of Circoviridae. PCVI is more closely related to banana bunch top virus (BBTV), a plant virus, than to chicken anemia virus (CAV) and beak and feather disease virus (BFDV) (both of which are avian circoviruses).

The gene map of isolate PCVII 412 is shown in FIG. 1. There are a total of six potential ORFs encoding proteins larger than 50 amino acid residues. A comparison between PCVII 412 and PK15 PCVI revealed homologies in four of the ORFs (Table 2). The function of the 35.8 kd, namely the putative DNA replicase protein, has been previously predicted (Meehan et al. (1997) J. Gen. Virol. 78:221-227). Analysis of these proteins predicted that both of the 35.8 kd and the antisense 27.8 kd proteins are nuclear proteins. Nucleotide sequence analysis also indicated that the start codons for the two proteins are within 33 bases of the origin of replication, which could also be the promoter. In addition, both ORFs ended with legitimate stop codons and poly A tail signals. Since some of the predicted proteins (based on size) could be found in western blots, these findings suggest that porcine circoviral mRNA can be transcribed from both senses of the replicated forms. However, there is no coding sequence long enough to code for the common 31 kd protein and the additional 20 kd protein for the PCVII 412 isolate detected by western blot analysis. This suggests that post-translational cleavage and/or RNA splicing may be involved in the expression of some of the porcine circovirus proteins.

TABLE 2
Putative Amino Acid Sequence Comparison Between
PK15 PCVI and PCVII 412

| | | Sequence | |
|---|---|---|---|
| Open reading frames | | Homology | Predicted |
| PCVI | PCVII/412 | % PCVI/412 | Localization and Function |
| 47-983 | 51-992 | 83.5 | Nucleus, putative Rep protein |
| (ORF 1) | (ORF 1) | | |
| 1723-1024 | 1735-1037 | 66.4 | Nucleus |
| (ORF 6) | (ORF 6) | | |
| 552-207 | 565-389 | 40.9 | Endoplasmic Reticulum |
| (ORF 4) | (ORF 3) | | |
| 658-40 | 671-359 | 29.1 | Microbody |
| (ORF 3) | (ORF 2) | | |

### Example 6

### Purification of PCVII Using Molecular Cloning Method

Dulac cells were infected with porcine retrovirus that is also found in many pig origin cell lines. In addition, other porcine pathogens were also found inconsistently associated with PCVII in PMWS-affected piglets. Thus, to obtain pure PCVII cultures, genetically cloned PCVII DNA was transferred to the susceptible non-porcine origin Vero cells using liposomes. After two passages, amplified PCV antigens were detected in the cells. The PCVII was seen to replicate and accumulate in the nuclei and was released into cytoplasm and other cells during cell mitosis.

### Example 7

### Multiplex PCR in PCVII Identification and PMWS Diagnosis

In order to differentiate the two strains of porcine circoviruses, PCVI and PCVII, two sets of primers were designed based upon the comparative analysis of the viral DNA sequences. The PCV group-specific pair of 1710+/850, and isolate PCVII 412 strain-specific 1100+/1570−, were used in multiplex PCR for testing field samples. These primer sets were used with frozen tissues and buffy coat cells of peripheral blood. As judged by the multiplex PCR, using those primer sets, not only was PCVII infection identified in these samples but the genetic relatedness of the field samples was also determined. The presence of circovirus was later confirmed by electron microscopy.

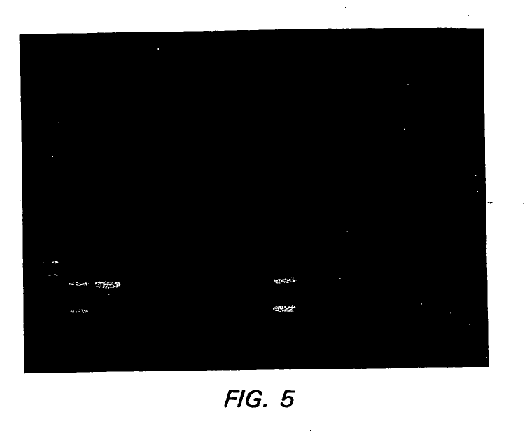

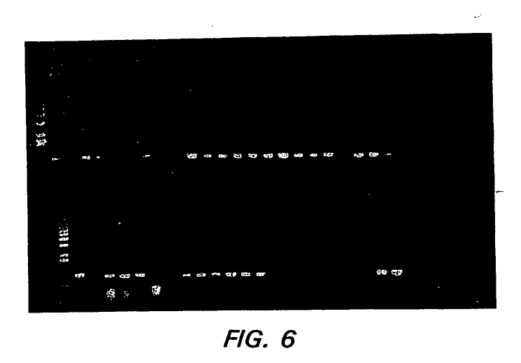

The potency of this diagnostic method was further tested with another group of samples collected from a PMWS-affected herd (see FIG. 5). The PCVII DNA sequences could also be identified in almost all the tissues in PMWS-affected piglets (FIG. 6).

### Example 8

### PCVII Viremia Prior to and During PMWS Outbreak

The development of PCR using serum enabled us to test the PCVII viremia in a swineherd showing specific anti-PCVII antibody. A group of 23 piglets was monitored from the age of one day until seven weeks and samples were collected at approximately two week intervals. A full-course of PCVII viremia and PMWS outbreak were observed, as indicated by the appearance to disappearance of the PCVII viremia which was detected in 9 of the 23 piglets. Most of piglets which showed PCVII viremia developed PMWS with some exhibiting severe PMWS. Table 3 shows the manifestation of PMWS in a typical pig. Gross lesions were found in most organs and tissues (Table 3).

TABLE 3
Clinical, Histological, Virological and Immunological Report
of a Typical PMWS Affected Piglet

| PMWS Pig | Gross appearance | Histopath | PCR |
|---|---|---|---|
| H254 | Spine, hairy, disinterested and wobbled | ND | ND |
| Saliva | ND | ND | + |
| Urine | Pale/clear | ND | + |
| Bile | Thin, not viscid | ND | + |
| Feces | Scant but normal | ND | + |
| Serum | Normal | ND | + |
| Plasma | Yellow | | + |
| Skin | Hint of yellow | | + |
| Fat | Little/no fat | | + |
| Muscle | Normal | | + |
| Tongue | Normal | Glossitis | + |
| Tonsil | Small crypts | Lymphocyte depletion | + |
| Cerv. LN | Enlarged | Lymphocyte depletion | + |
| Med. LN | Very large, dark surface, yellow center | Lymphocyte depletion | + |
| Mesenteric LN | Very enlarged, dark and wet | Lymphocyte depletion | + |
| Inguinal LN | Large, dark and wet | Lymphocyte depletion | + |
| Spleen | Small and thin | Lymphocyte depletion | + |
| Thymus | Small and difficult to find | ND | + |
| Treachea | Normal | Metaplasia adenitis | + |
| Lung | A, M lobes 80% atelectasis; firm | Interstitial Pneumonia | + |
| | texture mottles and spots thoughout all | | + |
| Heart | lobes | | + |
| Liver | Thin and flabby | | + |
| Gall Bladder | “Camouflage” pattem mottling | | + |
| Pancreas | Normal, moderately full | | + |
| Adrenal | Normal | Focal adrenalitis | + |
| Brain | Normal | Meningitis | + |
| Eye | Normal | | + |
| Stomach | Normal, white sclera | | + |
| Small intestine | Normal, full of feed | Peyers Patch | + |
| Large intestine | Normal | Submucosal inflam | + |
| Kidney | Normal, sandy/gritty contents | Interstitial nephritis | + |
| Urinary bladder | Enlarged, dark and no pus | | + |
| | Normal | | |
| | | Ref mg × 10⁹/L | |
| CBC | WBC: 20.1 | 11.0-22.0 | |
| | Segs: 62% or 12.462 | 3.08-10.4 | |
| | Lymphs: 29.0% or 5.829 | 4.29-13.6 | |
| FACS | CD3: 52.1% | 55% | |
| | CD4: 9.0% | 30% | |
| | CD8: 66.5% | 15% | |

### Example 9

### Host Immune System Dysfunction in PMWS Affected Piglets

While lymphocyte infiltration was discovered in most of the tissues, lymphocyte depletion was consistently found in all the lymphoid tissues (Table 4). Decreased CD4 cell, and increased CD8 cells were also seen, while CD3 cells remained relatively stable (Table 4, mean numbers are from two PMWS affected and 40 negative control piglets). These changes resulted in CD4/CD8 ratio that drastically dropped from 1.58 to 0.13. These finding suggested that PCVII could induce host immune system malfunction and therefore suppress the host immune responses to PCVII and possibly other pathogens. Thus, PMWS appears to be a disease of immunodeficiency in piglets.

TABLE 4
Lymphocyte Surface Markers of PMWS Affected
and Control 6-week-old Piglets

| | CD3 | CD4 | CD8 | CD4/CD8 Ratio |
|---|---|---|---|---|
| PMWS | 59.88 | 8.85 | 67.6 | 0.13 |
| Control | 53.46 | 24.02 | 15.18 | 1.58 |

Deposits of Strains Useful in Practicing the Invention

A deposit of biologically pure cultures of clone B9WTA, a clone including the full-length nucleic acid sequence of PCVII B9 as depicted in FIGS. 4A-4B, was made with the American Type Culture Collection, 10801 University Boulevard, Manassas, Va. on ______ and assigned Accession No. ______.

The accession number indicated was assigned after successful viability testing, and the requisite fees were paid. The deposits were made under the provisions of the Budapest Treaty on the International Recognition of the Deposit of Microorganisms for the Purpose of Patent Procedure and the Regulations thereunder (Budapest Treaty). This assures maintenance of viable cultures for a period of thirty (30) years from the date of deposit. The organisms will be made available by the ATCC under the terms of the Budapest Treaty, which assures permanent and unrestricted availability of the progeny to one determined by the U.S. Commissioner of Patents and Trademarks to be entitled thereto according to 35 U.S.C.§122 and the Commissioner's rules pursuant thereto (including 37 C.F.R. §1.12 with particular reference to 886 OG 638). Upon the granting of a patent, all restrictions on the availability to the public of the deposited cultures will be irrevocably removed.

These deposits are provided merely as convenience to those of skill in the art, and are not an admission that a deposit is required under 35 U.S.C. §112. The nucleic acid sequences of these genes, as well as the amino acid sequences of the molecules encoded thereby, are incorporated herein by reference and are controlling in the event of any conflict with the description herein.

### Example 10

### Culture and Isolation of Porcine Circovirus Strains

Tissue samples were collected in France, Canada and the USA from lung and lymph nodes of piglets. These piglets exhibited clinical signs typical of the post-weaning multisystemic wasting syndrome. To facilitate the isolation of the viruses, tissue samples were frozen at −70° C. immediately after autopsy.

Viruses 1103 and 1021 were isolated respectively in Alberta, respectively Saskatoon, Canada, from abortive cases according to the method described in Ellis et al., (1998) Can. J. Vet. 39: 44-51.

For the viral isolation, suspensions containing about 15% tissue sample were prepared in a minimum medium containing Earle's salts (EMEM, BioWhittaker UK Ltd., Wokingham, UK), penicillin (100 IU/ml) and streptomycin (100 μg/ml) (MEM-SA medium), by grinding tissues with sterile sand using a sterile mortar and pestle. This ground preparation was then taken up in MEM-SA, and then centrifuged at 3000 g for 30 minutes at +4° C. in order to harvest the supernatant.

Prior to the inoculation of the cell cultures, a volume of 100 μl of chloroform was added to 2 ml of each supernatant and mixed continuously for 10 minutes at room temperature. This mixture was then transferred to a microcentrifuge tube, centrifuged at 3000 g for 10 minutes, and then the supernatant was harvested. This supernatant was then used as inoculum for the viral isolation experiments.

All the viral isolation studies were carried out on PK15 cell cultures, known to be uncontaminated with the porcine circovirus (PCV), pestiviruses, porcine adenoviruses and porcine parvoviruses (Allan et al., (1995) Vet. Microbiol. 44: 49-64).

The isolation of the porcine circoviruses was carried out according to the following technique: Monolayers of PK15 cells were dissociated from confluent cultures by trypsinization with a trypsin-versene mixture and taken up in MEM-SA medium containing 15% fetal calf serum not contaminated by pestivirus (=MEM-G medium) in a final concentration of about 4×10⁵ cells per ml. aliquot fractions of 10 mls each of this cell suspension were then mixed with 2 ml aliquot fractions of the inocula described above, and the final mixtures were aliquoted in 6 ml volumes in two Falcon flasks of 25 cm². These cultures were then incubated at 37° C. for 18 hours under an atmosphere containing 10% CO₂.

After incubation, the culture medium of the semi-confluent monolayers were treated with 300 mM D-glucosamine (Cat # G48175, Sigma-Aldrich Company Limited, Poole, UK) (Tischr I. et al., (1987) Arch. Virol. 96:39-57), then incubation was continued for an additional period of 48-72 hours at 37° C. One of the two Falcons of each inoculum was then subjected to 3 successive freeze/thaw cycles. The PK15 cells of the remaining Falcon were treated with a trypsin-versene solution, resuspended in 20 ml of MEM-G medium, and then inoculated into 75 cm² Falcons at a concentration of 4×10⁵ cells/ml. The freshly inoculated flasks were then “superinfected” by addition of 5 ml of the corresponding lysate obtained after the freeze/thaw cycles.

### Example 11

### Preparation of Samples of Cell Culture for the Detection of Porcine Circoviruses by Immunofluorescence or by In Situ Hybridization

A volume of 5 ml of the “superinfected” suspension was collected and inoculated into a Petri dish 55 mm in diameter containing a sterile and fat-free glass coverslip. The cultures in the flasks and on glass coverslips were incubated at 37° C. and treated with glucosamine as described in Example 1. The cultures on glass coverslips were harvested from 24 to 48 hours after the treatment with glucosamine and fixed, either with acetone for 10 minutes at room temperature, or with 10% buffered formaldehyde for 4 hours. Following this fixing, all the glass coverslips were stored at −70° C., on silica gel, before their use for the in situ hybridization studies and the immunocytochemical labelling studies.

### Example 12

### Techniques for the Detection of PCV Sequences by In Situ Hybridization

In situ hybridization was carried out on tissues collected from diseased pigs and fixed with formaldehyde and also on the preparations of cell cultures inoculated for the viral isolation (see Example 3) and fixed on glass coverslips.

Complete genomic probes corresponding to the PK15 porcine circoviruses (PCV) and to the infectious chicken anaemia virus (CAV) were used. The plasmid pPCV1, containing the replicative form of the PCV genome, cloned in the form of a single 1.7 kilobase pair (kbp) insert (Meehan B. et al. (1997) J. Gen. Virol. 78: 221-227), was used as specific viral DNA source for PCV. An analogous plasmid, pCAA1, containing the 2.3 kbp replicative form of the avian circovirus CAV was used as negative control. The respective glycerol stocks of the two plasmids were used for the production and purification of the plasmids according to the alkaline lysis technique (Sambrook J. et al., Molecular cloning: A Laboratory Manual. 2nd Edition, Cold Spring Harbor Laboratory, Cold Spring Harbor, N.Y., 1989) so that they are then used as templates for the preparation of the probes. The circovirus probes representative of the complete genomes of PCV and of CAV were produced from the purified plasmids described above (1 μg for each probe) and from hexanucleotide primers at random using a commercial nonradioactive labelling kit (“DIG DNA labelling kit”, Boehringer Mannheim, Lewes, UK) according to the supplier's recommendations. The digoxigenin-labelled probes were taken up in a volume of 50-100 μl of sterile water before being used for the in situ hybridization.

The diseased pig tissue samples, enclosed in paraffin and fixed with formaldehyde, as well as the preparations of infected cell cultures, fixed with formaldehyde, were prepared for the detection of the PCV nucleic acids according to the following technique:

Sections 5 μm thick were cut from tissue blocks enclosed in paraffin, rendered paraffin free, and then rehydrated in successive solutions of alcohol in decreasing concentrations. The tissue sections and the cell cultures fixed with formaldehyde were incubated for 15 minutes and 5 minutes respectively at 37° C. in a 0.5% proteinase K solution in 0.05 M Tris-HCl buffer containing 5 mM EDTA (pH 7.6). The slides were then placed in a 1% glycine solution in autoclaved distilled water, for 30 seconds, washed twice with 0.01 M PBS buffer (phosphate buffered saline) (pH 7.2), and finally washed for 5 minutes in sterile distilled water. They were finally dried in the open air and placed in contact with the probes.

Each tissue/probe preparation was covered with a clean and fat-free glass coverslip, and then placed in an oven at +90° C. for 10 minutes, and then placed in contact with an ice block for 1 minute, and finally incubated for 18 hours at 37° C. The preparations were then briefly immersed in a 2× sodium citrate salt (SSC) buffer (pH 7.0) in order to remove the protective glass coverslips, and then washed twice for 5 minutes in 2×SSC buffer and finally washed twice for 5 minutes in PBS buffer.

After these washes, the preparations were immersed in a solution of 0.1 M maleic acid, 0.15 M NaCl (pH 7.5) (maleic buffer) for 10 minutes, and then incubated in a 1% solution of blocking reagent (Cat # 1096176, Boehringer Mannheim UK, Lewis, East Sussex, UK) in maleic buffer for 20 minutes at 37° C.

The preparations were then incubated with a 1/250 solution of an anti-digoxigenin monoclonal antibody (Boehringer Mannheim), diluted in blocking buffer, for 1 hour at 37° C., washed in PBS and finally incubated with a biotinylated anti-mouse immunoglobulin antibody for 30 minutes at 37° C. The preparations were washed in PBS and the endogenous peroxidase activity was blocked by treatment with a 0.5% hydrogen peroxide solution in PBS for 20 minutes at room temperature. The preparations were again washed in PBS and treated with a 3-amino-9-diethylcarbazole (AEC) substrate (Cambridge Bioscience, Cambridge, UK) prepared immediately before use.

After a final wash with tap water, the preparations were counterstained with hematoxylin, “blued” under tap water, and mounted on microscope glass coverslips with a mounting fluid (GVA Mount, Cambridge Bioscience, Cambridge, UK). The experimental controls included the use of a nonpertinent negative probe (CAV) and of a positive probe (PCV) on samples obtained from diseased pigs and from nondiseased pigs.

### Example 13

### Technique for the Detection of PCV by Immunofluorescence

The initial screening of all the cell culture preparations fixed with acetone was carried out by an indirect immunofluorescence technique (IIF) using a 1/100 dilution of a pool of adult pig sera. This pool of sera comprises sera from 25 adult sows from Northern Ireland and is known to contain antibodies against a wide variety of porcine viruses, including PCV: porcine parvovirus, porcine adenovirus, and PRRS virus. The IIF technique was carried out by bringing the serum (diluted in PBS) into contact with the cell cultures for one hour at 37° C., followed by two washes in PBS. The cell cultures were then stained with a 1/80 dilution in PBS of a rabbit anti-pig immunoglobulin antibody conjugated with fluorescein isothiocyanate for one hour, and then washed with PBS and mounted in glycerol buffer prior to the microscopic observation under ultraviolet light.

### Example 14

### Results of the In Situ Hybridization on Diseased Pig Tissues

The in situ hybridization, using a PCV genomic probe, prepared from tissues collected from French, Canadian and Californian piglets having multisystemic wasting lesions and fixed with formaldehyde, showed the presence of PCV nucleic acids associated with the lesions, in several of the lesions studied. No signal was observed when the PCV genomic probe was used on tissues collected from nondiseased pigs or when the CAV probe was used on the diseased pig tissues. The presence of PCV nucleic acid was identified in the cytoplasm and the nucleus of numerous mononuclear cells infiltrating the lesions in the lungs of the Californian piglets. The presence of PCV nucleic acid was also demonstrated in the pneumocytes, the bronchial and bronchiolar epithelial cells, and in the endothelial cells of the arterioles, the veinlets and lymphatic vessels.

In diseased French pigs, the presence of PCV nucleic acid was detected in the cytoplasm of numerous follicular lymphocytes and in the intrasinusoidal mononuclear cells of the lymph nodes. The PCV nucleic acid was also detected in occasional syncytia. Depending on these detection results, samples of Californian pig lungs, French pig mesenteric lymph nodes, and Canadian pig organs were selected for the purpose of isolating new porcine circovirus strains.

### Example 15

### Results of the Cell Culture of the New Porcine Circovirus Strains and Detection by Immunofluorescence

No cytopathic effect (CPE) was observed in the cell cultures inoculated with the samples collected from French piglets (Imp.1008 strain), Californian piglets (Imp.999 strain) and Canadian piglets (Imp.1010 strain) showing clinical signs of multisystemic wasting syndrome. However, immunolabelling of the preparations obtained from the inoculated cell cultures, after fixing using acetone and with a pool of pig polyclonal sera, revealed nuclear fluorescence in numerous cells in the cultures inoculated using the lungs of Californian piglets (Imp.999 strain), using the mediastinal lymph nodes of French piglets (Imp.1008 strain), and using organs of Canadian piglets (Imp.1010 strain).

### Example 16

### Extraction of the Genomic DNA of the Porcine Circoviruses

The replicative forms of the new strains of porcine circoviruses (PCV) were prepared using infected PK15 cell cultures (see Example 3) (10 Falcons of 75 cm²) harvested after 72-76 hours of incubation and treated with glucosamine, as described for the cloning of the replicative form of CAV (Todd et al., (1991) J. Clin. Microbiol. 29: 933-939). The double-stranded DNA of these replicative forms was extracted according to a modification of the Hirt technique (Hirt B. (1967) J. Mol. Biol. 36: 365-369), as described by Molitor (Molitor et al., (1984) Virology, 137: 241-254).

### Example 17

### Restriction Map of the Replicative Form of the Genome of the Porcine Circovirus Imp.999 Strain

The DNA (1-5 μg) extracted according to the Hirt technique was treated with S1 nuclease (Amersham) according to the supplier's recommendations, and then this DNA was digested with various restriction enzymes and the products of digestion were separated by electrophoresis on a 1.5% agarose gel in the presence of ethidium bromide as described by Todd et al., (1990) J. Gen. Virol. 71: 819-823). The DNA extracted from the cultures of the Imp.999 strain possess a unique EcoRI site, 2 SacI sites and do not possess any PstI site. This restriction profile is therefore different from the restriction profile shown by the PCV PK15 strain (Meehan B. et al., (1997) 78, 221-227) which possess in contrast a PstI site and no EcoRI site.

### Example 18

### Cloning of the Genome of the Porcine Circovirus Imp.999 Strain

The restriction fragment of about 1.8 kbp generated by digestion of the double-stranded replicative form of the PCV Imp.999 strain with the restriction enzyme EcoRI was isolated after electrophoresis on a 1.5% agarose gel (see Example 3) using a Qiagen commercial kit (QIAEXII Gel Extraction Kit, Cat # 20021, QIAGEN Ltd., Crawley, West Sussex, UK). This EcoRI-EcoRI restriction fragment was then ligated with the vector pGEM-7 (Promega, Medical Supply Company, Dublin, Ireland), previously digested with the same restriction enzymes and dephosphorylated, according to standard cloning techniques (Sambrook J. et al. Molecular cloning: A Laboratory Manual, 2nd Edition, Cold Spring Harbor Laboratory, Cold Spring Harbor, N.Y., 1989). The plasmids obtained were transformed into an Escherichia coli JM109 host strain (Stratagene, La Jolla, USA) according to standard techniques. The EcoRI-EcoRI restriction fragment of the PCV Imp.999 strain was also cloned into the EcoRI site of the vector pBlueScript SK+ (Stratagene Inc. La Jolla, USA). Among the clones obtained for each host strain, at least 2 clones containing the fragments of the expected size were selected. The clones obtained were then cultured and the plasmids containing the complete genome of the Imp.999 strain were purified in a small volume (2 ml) or in a large volume (250 ml) according to standard plasmid preparation and purification techniques.

### Example 19

### Sequencing of a Genomic DNA (Double-Stranded Replicative Form) of the PCV Imp.999 Strain

The nucleotide sequence of two EcoRI Imp.999 clones (clones pGEM-7/2 and pGEM-7/8) was determined according to Sanger's dideoxynucleotide technique using the sequencing kit “AmpliTaq DNA polymerase FS” (Cat # 402079 PE Applied Biosystems, Warrington, UK) and an Applied BioSystems AB1373A automatic sequencing apparatus according to the supplier's recommendations. The initial sequencing reactions were carried out with the M13 “forward” and “reverse” universal primers. The following sequencing reactions were generated according to the “DNA walking” technique. The oligonucleotides necessary for these subsequent sequencings were synthesized by Life Technologies (Inchinnan Business Park, Paisley, UK).

The sequences generated were assembled and analysed by means of the MacDNASIS version 3.2 software (Cat # 22020101, Appligene, Durham, UK). The various open reading frames were analysed by means of the BLAST algorithm available on the “National Center for Biotechnology Information” (NCBI, Bethesda, Md., USA) server.

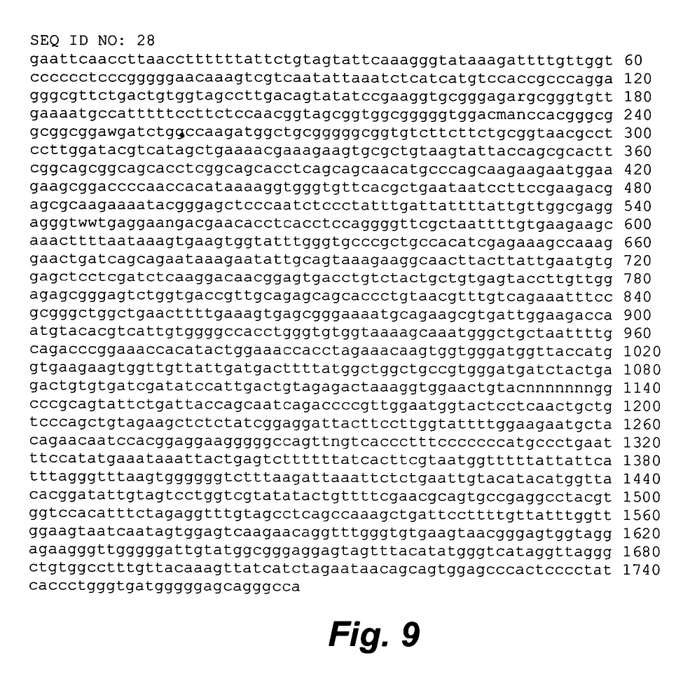

The complete sequence of the EcoRI--EcoRI fragments obtained initially from the clone pGEM-7/8 (SEQ ID NO: 28) is presented in FIG. 9. It starts arbitrarily after the G of the EcoRI site and exhibits a few uncertainties from the point of view of the nucleotides.

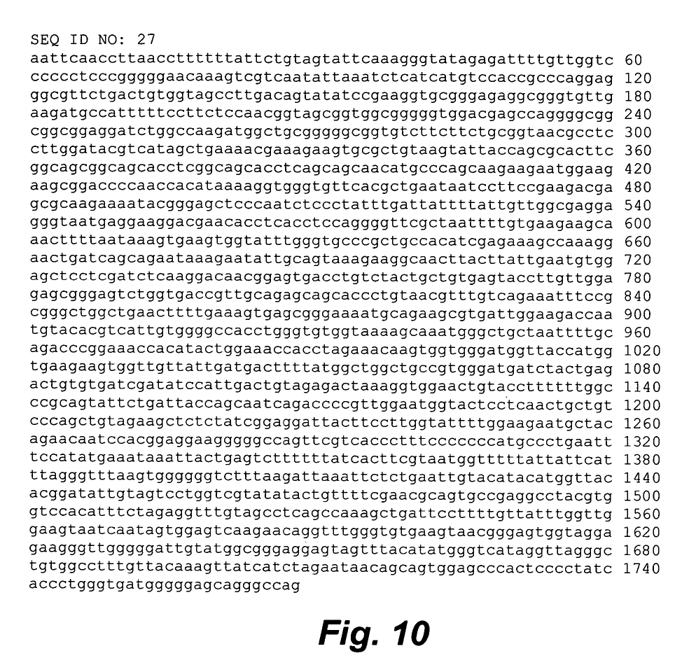

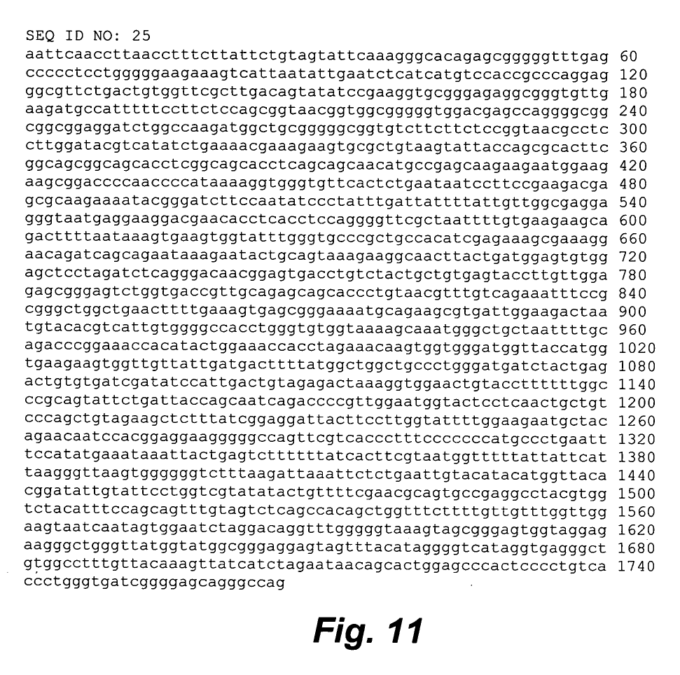

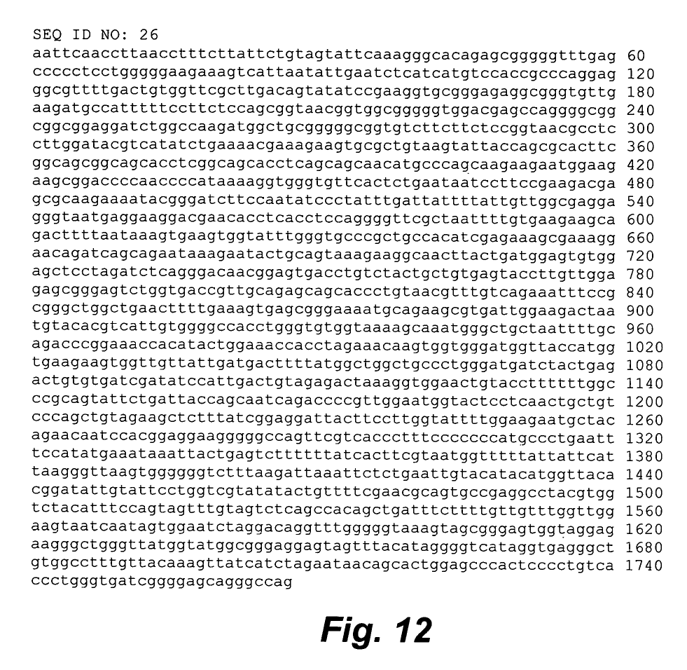

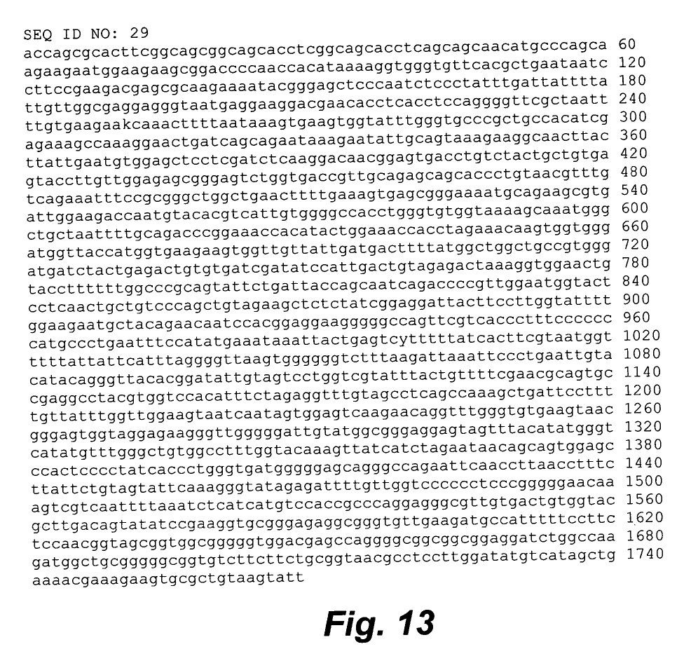

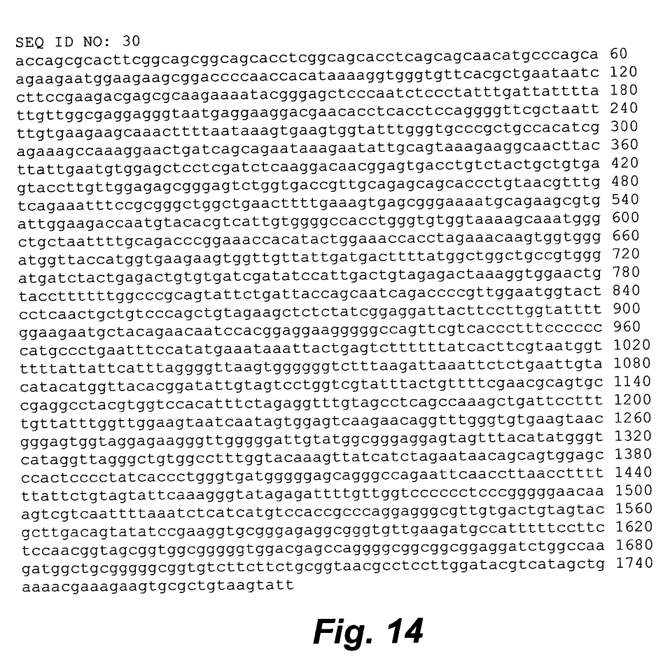

The sequencing was then optimized and the SEQ ID NO: 27 (FIG. 10) gives the total sequence of this strain, which was made to start arbitrarily at the beginning of the EcoRI site, that is to say the G as the first nucleotide. The procedure was carried out in a similar manner for obtaining the sequence of the other three isolates according to the invention (see SEQ ID NOS: 24-30.

The sizes of the genomes of the four strains are: Imp.1011-48121 (SEQ ID NO: 25), 1767 nucleotides; Imp.1011-48285 (SEQ ID NO: 26), 1767 nucleotides; Imp.999 (SEQ ID NO: 27), 1768 nucleotides; and Imp.1010 (SEQ ID NO: 24), 1768 nucleotides.

### Example 20

### Analysis of the Sequence of the PCV Imp.999 Strain

When the sequence generated from the Imp.999 strain was used to test for homology with respect to the sequences contained in the GenBank databank, the only significant homology which was detected is a homology of about 76% (at nucleic acid level) with the sequence of the PK15 strain (accession numbers Y09921 and U49186) (see FIG. 5).

At the amino acid level, the test for homology in the translation of the sequences in the 6 phases with the databanks (BLAST X algorithm on the NCBI server) made it possible to demonstrate a 94% homology with the open reading frame corresponding to the theoretical replicase of the BBTV virus similar to the circoviruses of plants (GenBank identification number 1841515) encoded by the GenBank U49186 sequence.

No other sequence contained in the databanks show significant homology with the sequence generated from the PCV Imp.999 strain.

Analysis of the sequences obtained from the Imp.999 strain cultured using lesions collected from Californian piglets having clinical signs of the multisystemic wasting syndrome shows clearly that this viral isolate is a new porcine circovirus strain.

### Example 21

### Comparative Analysis of the Sequences

The alignment of the nucleotide sequences of the 4 new PCV strains was made with the sequence of the PCV PK15 (PCVI) strain (FIG. 5). A homology matrix taking into account the four new strains and the previous PK15 strain was established (Table 5).

TABLE 5
Homology matrix of four new strains and the PK15 (PCVI) strain

| | 1* | 2 | 3 | 4 | 5 |
|---|---|---|---|---|---|
| 1 | 1.0000 | 0.9977 | 0.9615 | 0.9621 | 0.7600 |
| 2 | | 1.0000 | 0.9621 | 0.9632 | 0.7594 |
| 3 | | | 1.0000 | 0.9949 | 0.7560 |
| 4 | | | | 1.0000 | 0.7566 |
| 5 | | | | | 1.0000 |

*1: Imp.1011-48121; 2: Imp.1011-48285; 3: Imp. 999; 4: Imp.1010; 5: PK15

The homology between the two French strains Imp.1011-48121 and Imp.1011-48285 is greater than 99% (0.9977). The homology between the two North American strains Imp.999 and Imp.1010 is also greater than 99% (0.9949). The homology between the French strains and the North American strains is slightly greater than 96%. The homology between all these strains and PK15 falls at a value between 75 and 76%.

It is deduced that the strains according to the invention are representative of a new type of porcine circovirus, distinct from the type represented by the PK15 strain. This new type, isolated from pigs exhibiting the PMWS syndrome, is called type II porcine circovirus, PK15 representing type I. The strains belonging to this type II exhibit remarkable nucleotide sequence homogeneity, although they have in fact been isolated from very distant geographical regions.

### Example 22

### Analysis of the Proteins Encoded by the Genome of the PCV Strains

The nucleotide sequence of the Imp.1010 isolate was considered to be representative of the other circovirus strains associated with the multi-systemic wasting syndrome. This sequence was analysed in greater detail with the aid of the BLASTX algorithm (Altschul et al. J. Mol. Biol. 1990. 215. 403-410) and of a combination of programs from the set of MacVector 6.0 software (Oxford Molecular Group, Oxford OX44GA, UK). It was possible to detect 13 open reading frames (or ORFs) of a size greater than 20 amino acids on this sequence (circular genome). These 13 ORFs are shown in Table 6.

TABLE 6
ORFs identified in the genome sequence of PCVII strain 1010

| | Size of the ORF | | | Size of Protein | |
|---|---|---|---|---|---|
| Name | Start | End | Strand | (nucleotides) | (amino acids) |
| ORF1 | 103 | 210 | sense | 108 nt | 35 aa |
| ORF2 | 1180 | 1317 | sense | 138 nt | 45 aa |
| ORF3 | 1363 | 1524 | sense | 162 nt | 53 aa |
| ORF4 | 398 | 1342 | sense | 945 nt | 314 aa |
| ORF5 | 900 | 1079 | sense | 180 nt | 59 aa |
| ORF6 | 1254 | 1334 | sense | 81 nt | 26 aa |
| ORF7 | 1018 | 704 | antisense | 315 nt | 104 aa |
| ORF8 | 439 | 311 | antisense | 129 nt | 42 aa |
| ORF9 | 190 | 101 | antisense | 90 nt | 29 aa |
| ORF10 | 912 | 733 | antisense | 180 nt | 59 aa |
| ORF11 | 645 | 565 | antisense | 81 nt | 26 aa |
| ORF12 | 1100 | 1035 | antisense | 66 nt | 21 aa |
| ORF13 | 314 | 1381 | antisense | 702 nt | 213 aa |

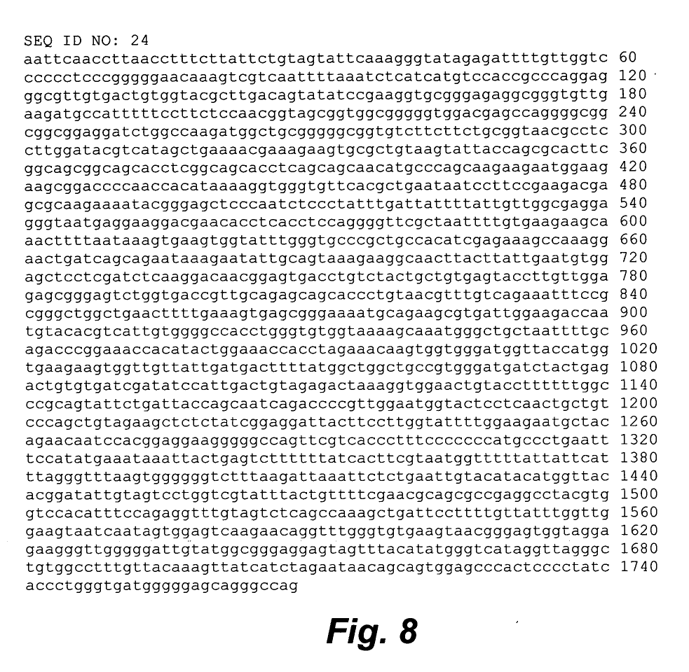

The positions of the start and end of each ORF refer to the sequence presented in FIG. 8 (SEQ ID No. 24), of the genome of strain 1010. The limits of ORFs 1 to 13 are identical for strain 999. They are also identical for strains 1011-48121 and 1011-48285, except for the ORFs 3 and 13 (ORF3: 1432-1539, sense, 108 nt, 35aa; ORF13: 314-1377, antisense, 705 nt, 234 aa).

Among these 13 ORFs identified in the strain 1010, four have a significant homology with analogous ORFs situated on the genome of the cloned virus PCV PK15 (PCVI). Each of the open reading frames present on the genome of all the circovirus isolates associated with the multisystemic wasting syndrome was analysed. These 4 ORFs are in Table 7.

TABLE 7
ORFs of PCVII strain 1010 identified in PCVI

| | | | | Size of Protein | |
|---|---|---|---|---|---|
| | Size of the ORF | | | amino | Molecular |
| Name | Start-End* | Strand | nucleotides | acids | mass |
| ORF4 | 398-1342 | sense | 945 nt | 314 aa | 37.7 kDa |
| ORF7 | 1018-704 | antisense | 315 nt | 104 aa | 11.8 kDa |
| ORF10 | 912-733 | antisense | 180 nt | 59 aa | 6.5 kDa |
| ORF13 | 314-1381 | antisense | 702 nt | 233 aa | 27.8 kDa |

*The positions of the start and end of each ORF refer to the sequence presented in FIG. 4 (SEQ ID No. 4). The size of the ORF (in nucleotides = nt) includes the stop codon.

The ORFs are defined with respect to strain Imp1010. The invention also encompasses the use of the corresponding ORFs in any other PCVII strain, and any of the PCVII strains as defined herein or in documents cited herein. Thus, from the genomic nucleotide sequence, it is routine art to determine the ORFs using a standard software, such as MacVector®. Also, alignment of genomes with that of strain 1010 and comparison with strain 1010 ORFs allows the one skilled in the art to readily determine the ORFs on the genome for another strain (e.g. those disclosed in WO-A-99 18214, say Imp 1008, Imp 1011-48121, Imp 1011-48285, Imp 999, as well as the new strains 1103 and 1121). Using software or making alignment is not undue experimentation and directly provides access to equivalent ORFs.

For example, the corresponding ORFs of strains 1103 and 1121 are as given in Table 8.

TABLE 8
ORFs of PCVII strains 1103 and 1121

| | | | | Size of the ORF | Protein Size |
|---|---|---|---|---|---|
| Name | Start | End | Strand | (nucleotides (nt)) | (amino acids (aa)) |
| ORF1 | 1524 | 1631 | Sense | 108 nt | 35 aa |
| ORF2 | 833 | 970 | Sense | 138 nt | 45 aa |
| ORF3 | 1016 | 1177 | Sense | 162 nt | 53 aa |
| ORF4 | 51 | 995 | Sense | 945 nt | 314 aa |
| ORF5 | 553 | 732 | Sense | 180 nt | 59 aa |
| ORF6 | 907 | 987 | Sense | 81 nt | 26 aa |
| ORF7 | 671 | 357 | Antisense | 315 nt | 104 aa |
| ORF8 | 92 | 1732 | Antisense | 129 nt | 42 aa |
| ORF9 | 1611 | 1522 | Antisense | 90 nt | 29 aa |
| ORF10 | 565 | 386 | Antisense | 180 nt | 59 aa |
| ORF11 | 298 | 218 | Antisense | 81 nt | 26 aa |
| ORF12 | 753 | 688 | Antisense | 66 nt | 21 aa |
| ORF13 | 1735 | 1037 | Antisense | 702 nt | 213 aa |

The positions of ORFs 1-6 shown in FIG. 1, identified in the genome of the PCVII strain 412 (SEQ ID NO: 1) and numbered according to the sequence comparisons shown in FIGS. 4A-4C, are given in Table 9, which also includes the eqivalent ORFs as designated for the strain 1010 (above).

TABLE 9
ORFs of PCVII strain 412

| | Position with in the PCV | Equivalent ORF |
|---|---|---|
| | sequence (numbering | of PCVII |
| ORF | according to FIGS. 4A-4C | strain 1010 |
| 1 | 51-992 | 4 |
| 2 | 6710360 | 7 |
| 3 | 565-389 | 10 |
| 4 | 553-729 | 5 |
| 5 | 1016-1174 | 3 |
| 6 | 1735-1037 | 4 |

The comparison between the genomic organization of the PCV Imp.1010 and PCV PK15 isolates allowed the identification of 4 ORFs preserved in the genome of the two viruses. Table 10 below presents the degrees of homology observed.

TABLE 10
Derees of homology between equivalent ORFs of PCVII 1010 and PCVI

| ORF Imp.1010/ | |
|---|---|
| ORF PVC PK15 | Percentage homology |
| ORF4/ORF1 | 86.0% |
| ORF13/ORF2 | 66.4% |
| ORF7/ORF3 | 61.5% |
| | (at the level of the overlap (104 aa)) |
| ORF10/ORF4 | 83.0% |
| | (at the level of the overlap (59 aa)) |

The greatest sequence identity was observed between ORF4 Imp.1010 and ORF1 PK15 (86% homology). This was expected since this protein is probably involved in the replication of the viral DNA and is essential for the viral replication (Meehan et al. (1997) J. Gen. Virol. 78: 221-227; Mankertz et al. (1998) J. Gen. Virol. 79: 381-384).

The corresponding ORFs of PCVII strains 1103 and 1121 are as given in Table 11.

TABLE 11
ORFs of PCVII strains 1103 and 1121

| | | | | Size of the ORF | Protein Size |
|---|---|---|---|---|---|
| Name | Start | End | Strand | (nucleotides (nt)) | (amino acids (aa)) |
| ORF1 | 1524 | 1631 | Sense | 108 nt | 35 aa |
| ORF2 | 833 | 970 | Sense | 138 nt | 45 aa |
| ORF3 | 1016 | 1177 | Sense | 162 nt | 53 aa |
| ORF4 | 51 | 995 | Sense | 945 nt | 314 aa |
| ORF5 | 553 | 732 | Sense | 180 nt | 59 aa |
| ORF6 | 907 | 987 | Sense | 81 nt | 26 aa |
| ORF7 | 671 | 357 | Antisense | 315 nt | 104 aa |
| ORF8 | 92 | 1732 | Antisense | 129 nt | 42 aa |
| ORF9 | 1611 | 1522 | Antisense | 90 nt | 29 aa |
| ORF10 | 565 | 386 | Antisense | 180 nt | 59 aa |
| ORF11 | 298 | 218 | Antisense | 81 nt | 26 aa |
| ORF12 | 753 | 688 | Antisense | 66 nt | 21 aa |
| ORF13 | 1735 | 1037 | Antisense | 702 nt | 213 aa |

The sequence identity between ORF13 Imp.1010 and ORF2 PK15 is less strong (66.4% homology), but each of these two ORFs indeed exhibits a highly conserved N-terminal basic region that is identical to the N-terminal region of the major structural protein of the CAV avian circovirus (Meehan et al. Arch. Virol. 1992. 124. 301-319). Furthermore, large differences are observed between ORF7 Imp.1010 and ORF3 PK15 and between ORF10 Imp.1010 and ORF4 PK15. In each case, there is a deletion of the C-terminal region of the ORF7 and ORF10 of the Imp.1010 isolate when they are compared with ORF3 and ORF4 of PCV PK15. The greatest sequence homology is observed at the level of the N-terminal regions of ORF7:ORF3 (61.5% homology at the level of the overlap) and of ORF10:ORF4 (83% homology at the level of the overlap).

It appears that the genomic organization of the porcine circovirus is quite complex as a consequence of the extreme compactness of its genome. The major structural protein is probably derived from splicing between several reading frames situated on the same strand of the porcine circovirus genome. It can therefore be considered that any open reading frame (ORF1 to ORF13) as described in the table above can represent all or part of an antigenic protein encoded by the type II porcine circovirus and is therefore potentially an antigen which can be used for specific diagnosis and/or for vaccination. The invention therefore relates to any protein comprising at least one of these ORFs. Advantageously, the invention relates to a protein essentially consisting of ORF4, ORF7, ORF10 or ORF13.

### Example 23

### Infectious Character of the PCV Genome Cloned from the New Strains

The plasmid pGEM-7/8 containing the complete genome (replicative form) of the Imp.999 isolate was transfected into PK15 cells according to the technique described by Meehan et al., (1992) Arch. Virol. 124: 301-319). Immunofluorescence analysis (see Example 11) carried out on the first passage after transfection on noncontaminated PK15 cells have shown that the plasmid of the clone pGEM7/8 was capable of inducing the production of infectious PCV virus. The availability of a clone containing an infectious PCV genetic material allows any useful manipulation on the viral genome in order to produce modified PCV viruses (either attenuated in pigs, or defective) which can be used for the production of attenuated or recombinant vaccines, or for the production of antigens for diagnostic kits.

### Example 24

### Production of PCV Antigens by In Vitro Culture

The culture of the noncontaminated PK15 cells and the viral multiplication were carried out according to the same methods as in Example 1. The infected cells are harvested after trypsinization after 4 days of incubation at 37° C. and enumerated. The next passage is inoculated with 4×10⁵ infected cells per ml.

At the end of the viral culture, the infected cells are harvested and lysed using ultrasound (Branson Sonifier) or with the aid of a rotor-stator type colloid mill (UltraTurrax, IKA). The suspension is then centrifuged at 3700 g for 30 minutes. The viral suspension is inactivated with 0.1% ethyleneimine for 18 hours at 37° C. or with 0.5% beta-propiolactone for 24 hours at +28° C. If the virus titer before inactivation is inadequate, the viral suspension is concentrated by ultrafiltration using a membrane with a 300 kDa cut-off (Millipore PTMK300). The inactivated viral suspension is stored at +5° C.

### Example 25

### Preparation of Vaccine in the Form of an Emulsion Based on Mineral Oil

The vaccine was prepared according to the following formula:

| | |
|---|---|
| suspension of inactivated porcine circovirus: | 250 ml |
| Montanide .RTM. ISA 70 (SEPPIC): | 750 ml |

The aqueous phase and the oily phase were sterilized separately by filtration. The emulsion was prepared by mixing and homogenizing the ingredients with the aid of a Silverson turbine emulsifier. One vaccine dose contained about 10^{7.5} TCID50. The volume of one vaccine dose was 0.5 ml for administration by the intradermal route, and 2 ml for administration by the intramuscular route.

This vaccine was used in a vaccination programme against the multisystemic wasting syndrome in combination with the Parvovax® vaccine.

### Example 26

### Preparation of Vaccine in the Form of a Metabolizable Oil-Based Emulsion

The vaccine was prepared according to the following formula:

| | |
|---|---|
| suspension of inactivated porcine circovirus: | 200 ml |
| Dehymuls HRE 7 (Henkel): | 60 ml |
| Radia 7204 (Oleofina): | 740 ml |

The aqueous phase and the oily phase was sterilized separately by filtration. The emulsion was prepared by mixing and homogenizing the ingredients with the aid of a Silverson turbine emulsifier. One vaccine dose contained about 10^{7.5} TCID50. The volume of one vaccine dose was 2 ml for administration by the intramuscular route. This vaccine is used in a vaccination programme against the multisystemic wasting syndrome in combination with the Parvovax® vaccine.

### Example 27

### The Indirect Immunofluorescence Results in Relation to the US and French PCV Virus Strains and to the PK15 Contaminant with a Hyperimmune Serum (PCV-T), a Panel of Monoclonal Antibodies F99 Prepared from PK15 and a Hyperimmune Serum Prepared from the Canadian Strain (PCV-C)

TABLE 12
Immunofluoresence

| | VIRUS | | |
|---|---|---|---|
| | PK15 (PCVI) | USA | France |
| PCV-T antiserum | *6400 | 200 | 800 |
| PCV-C antiserum | 200 | *6.400 | *6.400 |
| F99 1H4 | *10 000 | <100 | 100 |
| F99 4B10 | *10 000 | <100 | <100 |
| F99 2B7 | *10 000 | 100 | <100 |
| F99 2E12 | *10 000 | <100 | <100 |
| F99 1C9 | *10 000 | <100 | 100 |
| F99 2E1 | *10 000 | <100 | <100 |
| F99 1H4 | *10 000 | 100 | <100 |

*Reciprocal of the last dilution of the serum or of the monoclonal antibody which gives a positive reaction in indirect immofluorescence.

### Example 28

### Experimental Production of the Porcine Multisystemic Wasting Syndrome-Protocol 1

Three-day old gnotobiotic piglets obtained by caesarean and kept in an isolating unit were inoculated with virus solutions of PCV. The type II PCV viruses used were the Imp 1010 isolate and the virus obtained from lymph node homogenates obtained from diseased pigs. Five groups were formed. The piglets were all inoculated at the age of three days by the oronasal route with 1.5 ml of virus solution according to the scheme shown in Table 13:

TABLE 13
Incoculation scheme

| Group | Number | Virus | Dose |
|---|---|---|---|
| A | 6 | Lymph node homogenate | ND |
| B | 5 | Imp.1010 (low passage) | 10² TCID50 |
| C | 4 | Imp.1010 (high passage) | 10² TCID50 |
| D | 2 | Lysate of PK15 cells free of | |
| | | PCV virus | |
| E | 3 | — | — |

Results of the experimental challenge: During the 5-week observation period, the piglets did not develop clinical signs, apart from one animal in group B which showed substantial exhaustion. At autopsy, the pigs in groups A, B and C exhibited hyperplasia of the lymph nodes (size 2 to 10 times greater than that for the animals in groups D and E), in particular of the submaxillary, bronchial, mesenteric, iliac and femoral ganglia. This hyperplasia was linked to a considerable expansion of the cortical zones by infiltration by monocytes and macrophages. The piglets in groups A, B and C also exhibited hyper-plasia of the bronchial lymphoid tissue. One piglet in each of groups A, B and C had pneumonia. The piglet in group B, which exhibited substantial exhaustion, and one piglet in group A had a gastric ulcer. Moreover, all the animals in groups A, B and C had myositis in the muscular tunica of the stomach and of the intestine. Most of the animals in groups A, B and C had myocarditis, multifocal hepatitis with lymphocyte, macrophage and eosinophile infiltration, as well as cortical and medullary interstitial nephritis. One piglet in group C had a liver whose size was bigger than normal, with disseminated clear foci at its surface. No lesion was observed in the piglets in groups D and E. Circovirus was isolated from the organs of pigs in groups A, B and C.

### Example 29

### Experimental Reproduction of the Porcine Multisystemic Wasting Syndrome-Protocols 2 and 3

Conventional piglets isolated from their mother from birth were inoculated with viral solutions of type II PCV, of porcine parvovirus, or with a mixture of the two viruses. The type II PCV viruses used were the Imp.1010 and Imp.1011 isolates (strain 48121). The PPV virus used is an isolate of Canadian origin, Imp.1005. This virus has a sequence (⅓ of the sequenced genome) that is identical to that of other known porcine parvovirus strains (PPV strain NADL-2 and Kresse strain). Two experimental protocols were carried out.

Protocol 2: Three groups were formed with 3-day-old piglets. The piglets were all inoculated by the oronasal route with 1 ml of viral solution according to the following scheme shown in Table 14.

TABLE 14
Innoculation Scheme

| Group | Number | Virus | Dose | |
|---|---|---|---|---|
| A | 5 | Imp.1010 | 10⁷ | TCID50 |
| B | 5 | Imp.1010 + Imp.1005 | 5 × 10⁶ | TCID50 |
| C | 2 | — | — | |
| (control) | | | | |

Results of the experimental challenge: Group A: 2 piglets died 21 days after the inoculation and one piglet was humanely killed 24 days after the inoculation. Group B: 1 piglet died 23 days after the inoculation and one piglet was humanely killed 24 days after the inoculation. The autopsies carried out on the piglets that died following an infection showed the presence of substantial macroscopic lesions: presence of fluid in the pleural cavity, lung oedema, haemorrhages in the kidneys, whitish lesions in the form of a pin head on the kidneys, hepatic necrosis. These lesions are identical to those observed in the field cases. The autopsies carried out on the sacrificed piglets did not show macroscopic lesions. The histological examinations performed on organs removed from the piglets in groups A and B which died following an infection, as well as in the sacrificed pigs in these 2 groups, showed a typical and complete pattern of the lesions of porcine multisystemic wasting syndrome which are observed in animals in the field: hepatic necrosis, necrosis of the lymph nodes, pancreatic necrosis, focal necrosis and severe haemorrhages in the kidneys, presence of syncytia in the lungs, severe necrosis of the hepatocytes with the presence of nuclear inclusions. It should be noted that a massive quantity of PCV antigen was found in all these lesions (dead or sacrificed pigs), but that the presence of PPV antigen could not be detected in these same lesions. No lesion could be detected in the control piglets in group C. Protocol 3: Four groups were formed with 4-week-old piglets. The pigs were all inoculated by the oronasal route with 1 ml of viral solution according to the scheme in Table 15.

TABLE 14
Innoculation Scheme

| Group | Number | Virus | Dose |
|---|---|---|---|
| A (control) | 2 | — | — |
| B | 4 | Imp.1005 (PPV) | 10^{5.3} TCID50 |
| C | 4 | Imp.1011 (PCV) | 10⁵ TCID50 |
| D | 4 | Imp.1005 + Imp.1011 | 10⁵ + 5 × 10⁴ TCID50 |

Results of the experimental challenge: 1 “control” piglet and 2 piglets in each experimental group (B, C and D) were humanely killed and subjected to autopsy 2 weeks after inoculation. Significant immunohistological lesions were observed in the two piglets in group D (PCV+PPV coinfection). It should be noted that it was not possible to detect the presence of porcine parvovirus in these lesions, although a seroconversion in relation to the porcine parvovirus was observed in all the pigs in group D. No macroscopic or histological lesion could be observed in the control piglet and in the piglets in the other groups. It therefore appears that the PCV+PPV combination makes it possible to reproduce histological lesions typical of the porcine multisystemic wasting syndrome. Following these two experimental protocols, it can be observed that the inoculation of PCV alone, as a PCV+PPV mixture, leads to a more or less severe reproduction of the porcine multisystemic wasting syndrome, but only the porcine circovirus can be detected in the lesions. By contrast, an experimental infection with PPV alone (group B of protocol 3) does not allow macroscopic or histological lesions to be induced; however, in the presence of PCV, the appearance of lesions is observed in 4-week-old pigs (group D of protocol 3).

### Example 30

### Myocarditis, Abortion and Intrauterine Infection Associated with PCVII

Late term abortions and farrowings with both stillborn and mummified piglets occurred in a new 450-female pig swine facility as it was brought into production. Pseudopregnancy was also observed in several gilts. Gilts received two doses of an inactivated vaccine containing parvovirus and leptospiral immunogens prior to breeding.

A litter received for postmortem examination consisted of nine fetuses that appeared to have died at various stages of gestation. There were 2 mummified, 2 macerated, 3 autolysed and 2 fresh, stillborn piglets. Lesions were observed on gross pathological examination in one partially autolysed fetus only. In this fetus both ventricles of the heart were dilated, the liver was enlarged and firm and there was both hydrothorax and ascites. Histopathologically, there were extensive areas of myocardial degeneration or necrosis with edema and mild fibrosis, and a diffuse moderate-infiltration of lymphocytes and macrophages. There was marked generalized hepatic congestion and hepatocellular loss. The spleen and kidneys were also congested. Significant histological lesions were not detected in the other fetuses.

Immunohistochemical staining for PCVII was performed as previously described using a rabbit polyclonal antiserum and a monoclonal antibody that were raised against PCVII. on sections of formalin-fixed, routinely processed and embedded tissue (Ellis et al., 1998; Ellis et al., 1999). In the fetus with dilated cardiomyopathy there was extensive staining for PCVII antigen throughout the affected myocardium. Staining was most extensive in areas of necrosis and appeared to involve primarily myocytes. Both cytoplasmic and nuclear staining was present. In multiple fetuses there was extensive staining in the liver. In some sections it appeared to involve primarily sinusoidal endothelium and Kupfer cells, while in other fetuses, including the one with myocarditis, there was also nuclear and cytoplasmic staining of hepatocytes. Positively stained cells were scattered throughout the lung, and multifocally in the kidney. Polymerase chain reaction for PCVII was performed as previously described using frozen tissue (Ellis et al., 1999). PCR product of the expected size for PCVII was amplified from fetal tissue. PCVII was isolated from the fetus with myocarditis and a pool of tissues from other fetuses in the litter by inoculating tissue homogenates onto PCV-free PK15 cells.

Fetal tissues were also examined for other viral pathogens that have been associated with fetal injury and abortions in swine, including, porcine parvovirus (PPV), porcine reproductive and respiratory syndrome virus (PRRSV), encephalomyocarditis (EMCV), and enteroviruses. PPV antigen was not detected by fluorescent antibody testing (FAT) on frozen sections of lung, liver, and spleen from the mummified or stillborn fetuses. Homogenates of liver, lung, and spleen from the aborted fetuses were also inoculated into cultures of PCV-free PK15 cells, primary porcine fallopian tube cells and Vero cells. Cytopathic viruses were not detected after three passages. Tissues were negative for PPV using PCR. PRRSV antigen was not detected by immunohistochemical staining.

Thus, there were fetal lesions and abortion directly associated with PCVII. These results also show vertical transmission of the virus.

In a previous study, PCVI was isolated from 2 of 160 pig fetuses examined, implying that this group of viruses can be vertically transmitted; however, PCVI antigen could not be associated with any lesions in the tissue (Allan et al., 1995). The exclusion of other agents that have been associated with fetal lesions and abortion in swine, including, PPV (Bolt et al., 1997; Molitor et al., 1991), PRRSV (Lager et al., 1996), EMCV (Kim et al., 1989), and enterovirus (Molitor et al., 1991) indicate that PCVII can cause significant fetal pathology and subsequent abortion.

However, PCVI immunogens (still according to the general definition given at the beginning) may elicit an immunogenic or protective response against myocarditis and/or abortion and/or intrauterine infection as well as post-weaning multisystemic wasting syndrome and ergo PCVI immunogens can also be used in the practice of this invention (e.g., in the methods, compositions, uses, etc.)—either alone or in conjunction with PCVII immunogens (the vector can contain and express DNA encoding for both a PCVI immunogen and/or epitope and a PCVII immunogen and/or epitope) and/or alone or in conjunction an immunogen and/or epitope of other porcine pathogen (if a vector is used, the vector can contain and express DNA encoding for both a PCVI immunogen and/or epitope and an immunogen and/or epitope of another porcine pathogen, or for a PCVI immunogen and/or epitope and a PCVII immunogen and/or epitope and an immunogen and/or epitope of another porcine pathogen). Thus, one skilled in the art may alternatively or additionally use a PCVI immunogen, and/or epitope and/or vector encoding such an immunogen and/or epitope in the practice of this invention without any undue experimentation; for instance, to so do, one need only read the text herein prior to this Example and at the conclusion of (after) this Example, and substitute --PCVI-- for “PCVII” with any modification minor based on teachings herein.

The wasting syndrome associated with PCVII infection most often occurs in 5-12 week old pigs (Allan et al., 1998; Ellis et al., 1998). Experimental infection of neonatal swine indicates a relatively long prodromal period between infection and the development of clinical signs associated with PCVII (Allan et al. 1999; Ellis et al. 1999). The findings herein show that the virus is transmitted vertically or in the perinatal period. Not only may interuterine vertical transmission of PCVII result in abortion, but it is possible that sublethally in utero-infected piglets may be the animals that subsequently develop PMWS.

Furthermore, these results show that inoculation of female pigs with a composition comprising an PCVII immunogen (which composition can also include an immunogen from another porcine pathogen, e.g., porcine parvovirus), prior to breeding or serving, or prior to the perinatal period and/or during gestation can prevent myocarditis and/or abortion and/or intrauterine infection associated with porcine circovirus-2, as well as post-weaning multisystemic wasting syndrome and other pathologic sequelae associated with PCVII, by eliciting an immunological response or antibodies against PCVII.

Of course, compositions, methods, and other aspects of the invention can be used or practiced in animals other than pigs, e.g., sheep, bison, cattle, wild boar; for instance, if PCVII infects such other animals.

The presence of PCVII in neonatal piglets suggests that vertical transmission may be an important means of viral transmission. This mode of transmission may be related not only to reproductive failure, but also to the development of multisystemic disease later in life. It is of interest to determine whether previously undetected PCVII (and PCVI) has been vertically transmitted in pork producing areas where PMWS, and by extension PCVII infection, has been endemic for at least several years.

Thirty-eight submissions involving reproductive failure received in the diagnostic laboratory at the Western College of Veterinary Medicine (WCVM), University of Saskatchewan, Saskatoon, Canada, over a four-year period from a total of 30 high health herds in Canada were evaluated. Five of the farms from which the samples were obtained had diagnosed cases of PMWS. Twenty-seven of the thirty-eight submissions (71%) were classified as abortions; five of these (13%) also involved at least one mummified fetus. Of the remaining 10 cases: 5 involved stillborn piglets along, with nonviable piglets (13%); 2 with stillborn and one or more mummified feti (5%); 2 with only stillborn piglets (5%); and one with only mummified feti (2.5%). Routine diagnostics for pathogens other than circovirus revealed 4 cases (11%) in which the etiology was determined to be porcine parvovirus and 2 cases (5%) in which the etiology was determined to be of bacterial origin. Gross necropsies were performed and tissues were collected and fixed in buffered formalin (fixation time 24-72 hrs) and, in most cases, fresh tissues were also submitted for routine microbiological evaluation. None of these cases had been previously tested for PCVII.

The PCR technique used for the detection of PCVI and PCVII was performed as previously described (Tischer et al. 1974). PCVI was not detected by PCR in any submissions comprising reproductive failure from the four-year period. PCVII was detected by PCR in two different submissions that originated from the same multi-site pork production unit on two separate occasions in the spring of the last year in the four-year period. The first of these submissions comprised a litter of piglets with gross evidence of myocarditis, cardiac hypertrophy, and chronic passive congestion.

Immunohistochemical identification of PCVII in tissues was performed as previously described (Tischer et al. 1974). Immunohistochemical staining (IHC) for PCVII was positive in hearts from all six of the piglets that were submitted, while 4 of 6 were positive by PCVII PCR (see following Table 16).

TABLE 16
Detection of PCVII in the formalin fixed hearts of porcine with
myocarditis by PCR, IHC and viral isolation in cell culture.

| PCVII postive tissues | PCR | IHC | Virus Isolation |
|---|---|---|---|
| Fixed | 5/6 | 6/6 | N/A |
| Frozen | 4/4 | N/A | 2/4 |

The second submission from the same farm consisted of a litter of four piglets in which 2 were stillborn and 2 others died shortly after birth. All four piglets also had gross evidence of a severe, difuse myocarditis, cardiac hypertrophy, and chronic passive congestion. Only fresh frozen heart, and pooled lung/spleen tissues were submitted for analysis. PCVII PCR was positive in the hearts of 2 of 4 piglets and in the pooled lung and splenic tissues of 4 of 4 piglets. Isolation of PCVII from affected hearts and/or pooled lung and splenic tissue was positive in 2 of the 4 cases that were PCVII positive by PCR. Based on serology and/or PCR, other agents associated with reproductive failure in swine, including porcine reproductive and respiratory syndrome virus and porcine parvovirus were apparently circulating in the breeding herd. However, these agents could not be shown to be associated with the severe cardiac (or other) lesions in the affected piglets, but they may contribute to PMWS.

PCVII was not detected by PCR or IHC in any representative cases of reproductive failure submitted during the first three years of the four-year period (it was detected in cases of reproductive failure submitted during the last year of the four-year period). In order to rule out damage to DNA due to formalin fixation as a possible adverse factor limiting the ability to detect PCVII by PCR, PCR was performed on tissues collected from four weanling piglets with PMWS, PCVII DNA was amplified in all fixed tissues tested, including; lung, liver, kidney and bronchial lymph node, from all four individuals. Moreover, the sensitivity of the PCR PCVII was independent of the length of time that each tissue was fixed in formalin.

These results confirm and extend the previous observation (West et al. 1999) that PCVII can be vertically transmitted and can be present in large amounts within lesions from piglets infected in utero. Vertical transmission of PCVII virus and resultant fetal damage, such as myocarditis, is an additional disease manifestation of PCVII. Furthermore, the failure to detect PCVII in cases of reproductive failure prior to the last year of the four-year period from an endemic area of PCVII infection may indicate that vertical transmission was not the primary mechanism responsible for the initial dissemination of viral infection. Sexual, as well as vertical, modes of transmission can be attributed to the spread of PCVII infection in pigs.

### Example 31

### Titration of PCVII

Titration is carried out in 96-well microplates. A suspension of PK15 cells (1.5×10⁵ cells per ml) is first introduced (100 μl per well). Then dilutions of the viral culture are done and 100 μl thereof are introduced in the wells. Incubation is done at 37° C. with CO₂. After 24 h, there is carried out a treatment with glucosamine for 30 minutes at 37° C. The culture medium is then removed and fresh medium is introduced. Incubation is conducted 72 h at 37° C. Revelation of the foci is done using an anti-PCVII monoclonal antibody and a FITC labelled mouse conjugate. This method can be used to titration for preparing inactivated as well as live attenuated PCVII.

### Example 32

### Vaccination of Piglets with DNA (Plasmid) Vector

Groups of 3 or 4 piglets, caesarian-derived day 0 are placed into isolators. The piglets are vaccinated day 2 either with (A) a plasmid comprising ORF 13 or with (B) a mixture of this plasmid and another plasmid comprising ORF 4, and with a physiological solution for the control group. Each plasmid is diluted in sterile physiological solution (NaCl 0.9%) at 250 μg/μl final concentration. A 2 ml volume is injected by intramuscular route in two points of 1 ml (1 point each side of the neck). A second injection of vaccine or placebo is administered day 14. Vaccination with DNA is well tolerated by piglets and no evidence for adverse reaction to vaccination is noted. The piglets are challenged day 21 by oronasal administration of PCVII viral suspension, 1 ml in each nostril. After challenge piglets are weighed once a week. Rectal temperatures are recorded on days 17, 21, 22, 24, 27, 29, 31, 34, 37, 41, 44. Day 44 fecal swabs are collected from each piglet for PCVII shedding. The virus is detected and quantified by quantitative PCR. Day 45 necropsies are performed and tissue samples are collected for virus isolation.

Clinical symptoms: There was no significant difference for the mean body weight gains or the mean body temperatures between groups.

Necropsy lesions: The only gross finding noted in pigs at termination was bronchial lymphadenopathy. The lesions are scored according the following criteria: 0=no visible enlargement of lymph nodes; 1=mild lymph nodes enlargement, restricted to bronchial lymph nodes; 2=moderate lymph nodes enlargement, restricted to bronchial lymph nodes; 3=severe lymph nodes enlargement, extended to bronchial submandibullar prescapsular and inguinal lymph nodes, as shown in Table 17.

TABLE 17

| | Lymphadenopathy | | |
|---|---|---|---|
| | scores | | |
| Groups | mean | std^{a} | N^{b} |
| (A) | 1.2 | 1.3 | 4 |
| (B) | 2.0 | 1.7 | 3 |
| controls | 3.0 | 0.0 | 3 |

^{a}std is an abbreviation for standard deviation;
^{b}N = number of piglets in each group

A reduction of the lymph node lesions is observed in 3 out 4 piglets immunized with (A) and 1 out 3 piglets immunized with (B) mixture. This difference was not significant (p>0.05) due to the high value of the standard deviations (std). Virus load in lymph nodes tissues: Quantitative virus re-isolation was performed on tissue homogenates prepared from bronchial and mesenteric lymph nodes. The data, shown in Table 18, corresponds to the virus titers in tissue homogenates after transformation in log₁₀.

TABLE 18
Virus Titers in Tissue Homogenates

PCVII titers

| | |
|---|---|
| Bronchial LN | Mesenteric LN |

| Groups | mean | std | mean | std | N |
|---|---|---|---|---|---|
| (A) | 0.9 | 0.8 | 0.9 | 0.8 | 4 |
| (B) | 0.7 | 0.6 | 0.2 | 0.2 | 3 |
| Controls | 2.0 | 1.1 | 1.8 | 1.1 | 4 |

Bronchial lymph nodes seem to contain the most infectious virus. A reduction of the viral load is observed in bronchial and mesenteric lymph nodes from piglets immunized with either (A) or (B) mixture. This reduction is significant (p # 0.05 for the plasmids mixture). Viral excretion: Post challenge fecal swabs are assessed for schedding PCVII by PCR based on amplification of PCVII ORF 13. Each assay (see Table 19) is performed in triplicate on 2 ml of sample. Unvaccinated controls are negative for PCVII prior challenge and positive after challenge confirming the validity of the PCR assay.

TABLE 19
Log₁₀ number of PCVII DNA molecules

| Groups | mean | std | N |
|---|---|---|---|
| (A) | 3.3 | 0.3 | 4 |
| (B) | 2.9 | 0.7 | 3 |
| Controls | 3.6 | 0.6 | 4 |

Values are expressed as log₁₀ (number of molecules of PCVII DNA in 2 μl sample). The differences between groups are not significant (p>0.05).

### Example 33

### Vaccination Outcome with PCVII-Vaccine

Vaccination was done using inactivated PCVII having a titer before inactivation of 10^{6.6} TCID50 formulated in an oil-in-water emulsion. The volume for one dose of vaccine was 2 ml.

Sixteen piglets (14 days old) were allocated in two groups of 8 animals each, one as a control group the other as a vaccinated group. The 8 animals of the vaccinated group were injected by intramuscular route at days 0 and 21. Four control group piglets were injected with a physiological solution. Vaccinated and control animals were then challenged day 35 by oronasal administration of a PCVII viral suspension, 5 ml in each nostril (5.5. DICC50 per nostril).

Antibodies production: Antibodies in sera was measured by immunofluorescence as shown in Table 20.

TABLE 20
Antibodies in Sera as Measured by Immunofluorescence

| | | Day | | | | | |
|---|---|---|---|---|---|---|---|
| | | 8* | Day 2* | Day 19 | Day 28 | Day 33 | Day 43 |
| vaccinated | mean | 2.5 | 2.2 | 1.9 | 2.7 | 2.8 | 2.9 |
| | titre | | | | | | |
| | std | 0.17 | 0.25 | 0.0 | 0.37 | 0.36 | 0.28 |
| control | mean | 2.4 | 2.3 | 1.9 | 1.9 | 1.9 | 1.9 |
| | titre | | | | | | |
| | std | 0.11 | 0.20 | 0.0 | 0.0 | 0.0 | 0.0 |

std = standard;
*residual titer of maternal antibodies at days −8 and −2 days

Vaccinated piglets seroconverted after the second vaccine injection. The difference between the vaccinated and control groups was significant (ANOVA analysis).

Viral excretion in feces: Rectal swabs were collected at different times after challenge to follow viral excretion. The faecal swabs are assessed by PCR for the presence of PCVII. Unvaccinated controls are negative for PCVII prior challenge and positive after challenge confirming the validity of the PCR assay.

Values (Table 21) are expressed as the percentage of piglets excreting PCVII in feces and as mean duration of excretion expressed in days.

TABLE 21
Viral excretion in feces

| | control | vaccinated |
|---|---|---|
| % viral excretion | 100% | 38% |
| Mean duration of excretion | 9.1 days | 1.3 days |

A reduction of viral excretion was observed in the vaccinated group. The difference between vaccinated and control piglets is significant (ANOVA analysis).

Virus load in lymph nodes tissues: Mediastinal and mesenteric lymph nodes were collected. Virus load (Table 22) was determined by immunochemistry.

TABLE 22
Virus load in lymph nodes tissues

| | | vaccinated | control |
|---|---|---|---|
| (1) | Mediastinal | 75% | 100% |
| | Mesenteric | 25% | 100% |
| (2) | Mediastinal | 0.4 | 1.9 |
| | Mesenteric | 0.1 | 1.9 |

(1) percentage of piglets having mediastinal or mesenteric lymph nodes from which it was possible to detect the presence of PCVII. (2) mean of scores using the following criteria: 0, lack of fluorescence; 1, some fluorescent foci on some organ slides; 2, approximately 1 foci per shot; 3, wholly fluorescent organ.

Values of (1) and (2) are lower in vaccinated piglets than in the controls. The differences were significant (Chi2 test for (1) and Kruskall-Wallis test for (2)).

Necropsy lesions: Necropsies at days 63 and 64 were performed and the lesions were scored (Table 23).

TABLE 23
Necropsy lesions

| | |
|---|---|
| Control | 16.8 |
| Vaccinated | 8.9 |

A significant reduction (Kruskall-Wallis test) was observed in the vaccinated group. The score (as shown in Table 24) for one piglet was equal to the sum of the scores corresponding to each organ observed.

TABLE 24
Lesions Scores

| | | | | | |
|---|---|---|---|---|---|
| skin color | normal | 0 | pleura | normal | 0 |
| | white | 1 | | lesion | 1 |
| | yellow | 2 | | visible | 2 |
| corpulence | normal | 0 | peritoneum | normal | 0 |
| | thin | 1 | | lesion | 1 |
| | very thin | 2 | stomach | normal | 0 |
| | cachecctic | 3 | | lesion | 1 |
| nucous | normal | 0 | | ulcer | 2 |
| | white | 1 | small intestine | normal | 0 |
| | yellow | 2 | | lesion | 1 |
| sub cut. | normal | 0 | large intestine | normal | 0 |
| Conjonctif | brilliant | 1 | | lesion | 1 |
| | yellow | 2 | Peyers plaques | normal | 0 |
| ganglions | normal | 0 | | visible on 1 part | 1 |
| (gg) | 1 large and/or | 1 | | of the intestine | |
| | congestive | | | visible on 2 part | 2 |
| | >1 large and/or | 2 | | of the intestine | |
| | congestive | | | very important | 3 |
| | >1 very large | 3 | liver | normal | 0 |
| thoracic | normal | 0 | | lesion | 1 |
| fluid | brilliant | 1 | kidney | normal | 0 |
| | visible | 2 | | lesion | 1 |
| heart | normal | 0 | bladder | normal | 0 |
| | lesion | 1 | | lesion | 1 |
| lungs | normal | 0 | | | |
| | lesion < A | 1 | | | |
| | lesion > 4 < 6 | 2 | | | |
| | lesion > 6 | 3 | | | |

Vaccination with inactivated PCVII, therefore, protected pigs against challenge as substantiated by significant reduction of viral excretion in feces, of virus load in organs and of lesions of PMWS.

### Example 34

### Vaccination of Piglets with Canarypox Live Vector and Results

Groups of 3 or 4 piglets, caesarian-derived day 0 were placed into isolators. Day 2 the piglets are vaccinated with 10⁸ pfu of (C) a canarypox comprising ORF 13, or (D) of a canarypox comprising ORF 13 and ORF 4, or parental canarypox, in 1 ml of PBS, by intramuscular route on the side of the neck. A second injection of vaccine or placebo was administered at day 14. Vaccination with canarypox was well tolerated by piglets and no evidence for adverse reaction to vaccination is noted. The piglets were challenged day 21 by oronasal administration of a PCVII viral suspension, 1 ml in each nostril. Day 45 necropsies were performed and samples of tissues are collected for virus isolation.

Necropsy results: PMWS is characterized generally by lymphadenopathy and more rarely by hepatitis or nephritis. Gross findings in lymph nodes were scored for each piglet in the following manner: 0=no visible enlargement of lymph nodes; 1=mild lymph nodes enlargement, restricted to bronchial lymph nodes; 2=moderate lymph nodes enlargement, restricted to bronchial lymph nodes; 3=severe lymph nodes enlargement, extended to bronchial, submandibullar prescapular and inguinal lymph nodes, as shown in Table 25.

TABLE 25
Lymph Nodes Score

| Groups | Scores |
|---|---|
| (C) | 0.5 |
| | 0.0 |
| | 0.0 |
| | 1.0 |
| mean | 0.38 |
| standard deviation | 0.48 |
| (D) | 0.0 |
| | 0.5 |
| | 0.5 |
| | 1.0 |
| mean | 0.5 |
| standard deviation | 0.41 |
| Controls | 2.0 |
| | 2.5 |
| | 2.5 |
| | 2.5 |
| mean | 2.38 |
| standard deviation | 0.25 |

Bronchial lymphadenopathy for PCVII is a prominent gross finding. A significant reduction of the lymph nodes lesion in relation to control group was observed after immunization with (C) and (D) (p<0.05).

### Example 35

### Animals and Methods for Vaccination with H. cerdo-Based Vaccines

The animals used in this study are selected conventional pigs that are PRRSV-free, and M. hyopneumoniae-free.

Conventional piglets will be selected while still with their mothers. All pigs will be weighed and their weights recorded. The pigs will be assigned to 4 groups of at least 5 pigs each, with stratification by weight, sex and litter of origin by Cerberus personnel. All pigs will be examined to ensure health status. Only clinically healthy animals will be included in the trial. All pigs will be identified by a sequentially numbered ear tag in the left ear. A new ear tag with the same identification number will be installed if an animal loses its ear tag.

Pigs will be vaccinated at 1 week and 2 weeks of age while with their mothers. Three groups of pigs will be vaccinated and a group left as unvaccinated control. Vaccinated animals will receive 1 dose (2 ml per dose) via the intramuscular route, as 0.5 ml over each shoulder and hip. Unvaccinated controls will not receive any injection.

At 3 weeks of age, all pigs will be weaned and challenged. Each group will be housed in separate pens in an isolation facility. The pigs will be necropsied at approximately 28 days post challenge. The negative control pigs will be necropsied at the final necropsy date. The experimental design is summarized in Table 26.

TABLE 26
Experimental Design

| Treatment Description | Helicobacter challenge | No challenge |
|---|---|---|
| Vaccine A | 5 pigs | |
| Vaccine B | 5 pigs | |
| Vaccine C | 5 pigs | |
| Negative Controls | | 5 pigs |

Challenge Procedure and evaluation: In case of severe clinical illness, treatments that are considered necessary for the animal's welfare may be administered. Each animal's ear tag number, date(s) of illness, presumptive diagnosis, treatment regimen, and disposition of the animal will be recorded. No treatment will be provided following challenge. A moribund or injured animal will be euthanized. An unhealthy animal (clinical illness or injury) may be withdrawn from the study.

Serology and Skin tests: Blood will be collected from the anterior vena cava prior to vaccination, prior to challenge and at necropsy. Helicobacter antibody levels will be determined using an. Antibodies against other pathogens might be assayed as needed. Skin tests will be performed.

Production Parameters: Pigs will be weighed upon arrival, prior to vaccination, prior to challenge and at each necropsy to evaluate potential weight gain or loss.

Necropsy: Pigs will be necropsied at 28 DPI as adapted to Helicobacter challenge.

### Example 36

### Method of Preparation of Helicobacter

Culture: Bacterial cultures were grown from glycerol (of vegetable origin) stocks, using a 5-10% inoculum into Brucella broth supplemented with 10% Fetal Bovine Serum (FBS). Cultures were grown in vented cap flasks in a triple gas incubator (85% N₂, 10% CO₂, 5% O₂) with shaking at about 70 rpm at 37° C. At the time of inoculation, TSA+5% Sheep's Blood (SB) plates were also struck as a diagnostic test to determine sample purity. Colonial morphology was punctiform and clear. Some hemolysis was seen on plates left in the incubator for 3-4 days. Cultures took 24-36 hours to grow to OD₆₀₀>1. Grown cultures were also struck on TSA+5% SB plates, and tested for catalase and urease production (Helicobacter cerdo was positive for both).

Centrifugation: Cultures grown to OD₆₀₀>1 were aliquoted into sterile centrifuge bottles and centrifuged at 7500 rpm for 20 minutes with a J10 Beckman rotor at approximately 8600 g. The supernatant was discarded and cell pellets were washed with 1× phosphate-buffered saline (PBS). Pellets were resuspended in PBS and aliquoted into 10-ml vaccine vials, and split into three different groups (A, B, and C) for different antigen preparations.

Lyophilization: Antigen preparations A and B were lyophilized for 36 hours with no stabilizers or preservatives. Well-formed cakes were observed.

Pepsin digestion: A pepsin solution (0.1%) was prepared in 10 mM HCl and filter sterilized twice with a 0.2 micron filter. Lyophilized antigen preparations A and B were digested with pepsin (1 μg of pepsin was added for every mg of dried cell mass) for 25 hours at 37° C. and gentle rocking. Samples of 100 μl were spread onto TSA+5% SB plates (incubated at 37° C. in a triple gas microaerophilic incubator) at 18 and 25 hours; no growth was seen after 96 hours, indicating that the pepsin digestion has an inactivation effect. The pepsin solution was also tested in the same manner. No growth was seen.

PBS neutralization: Antigen preparations A and B had a pH of about 2.0 after the pepsin digestion, so they were neutralized with a 2:1 volume of PBS. After pH neutralization, pH was about 7.0.

Sonication: Antigen preparation C was sonicated on ice by 30 second pulses on high with a 30 second cooling period, 20 times, using a probe type sonicator. Lysis was confirmed by microscopic examination, although an inactivation test (determined by plating 100 μl samples onto TSA+5% SB plates incubated at 37° C. in a triple gas microaerophilic incubator for 72 hours) revealed that some bacteria were still viable and capable of forming colonies.

Formalin inactivation: Antigen preparations B and C were formalin inactivated with 0.3% formalin with rocking for 24 hours at 37° C. Samples of 100 μL were spread onto TSA+5% SB plates (incubated at 37° C. in a triple gas microaerophilic incubator) at 18 and 24 hours, no growth was seen after 96. Antigen prep. B had a final formalin concentration of 0.11%. Antigen prep. C had a final formalin concentration of 0.04%.

### Example 37

### Formulations of Antigen Preparations Prepared from Helicobacter cerdo Treated with Pepsin or Formalin

Vaccines will be formulated, as shown in Table 27, extemporaneously by dissolving the lyophilized bacteria in 10 ml of adjuvant per vial. Values reflect the concentration of each ingredient after formulation.

TABLE 27
Vaccine Formulations

| | | | |
|---|---|---|---|
| | | | Ingredient |
| | | | Quantity mg |
| | | Ingredient Quantity | per dose |
| Ingredient | Components | mg per ml | (2 ml) |

Antigen A

| | | | |
|---|---|---|---|
| PBS | NaCl | 2 | 4 |
| | KCl | 0.05 | 0.1 |
| | Na₂HPO₄ | 0.2875 | 0.575 |
| | KH₂PO₄ | 0.05 | 0.1 |
| | deionized H₂O | | |
| pepsin (solution) | | 3.5 μg | 7.0 μg |
| | HCl | 0.042 | 0.084 |
| Helicobacter cells | | 3.5 | 7.0 |

Antigen B

| | | | |
|---|---|---|---|
| PBS | NaCl | 2 | 4 |
| | KCl | 0.05 | 0.1 |
| | Na₂HPO₄ | 0.2875 | 0.575 |
| | KH₂PO₄ | 0.05 | 0.1 |
| | deionized H2O | | |
| formalin | | 1.11 (0.11% formalin) | 2.22 |
| pepsin (solution) | | 3.7 μg | 7.4 μg |
| | HCl | 0.045 | 0.089 |
| Helicobacter cells | | 3.7 | 7.3 |

Antigen C:

| | | | |
|---|---|---|---|
| PBS | NaCl | 1.2 | 2.4 |
| | KCl | 0.03 | 0.06 |
| | Na₂HPO₄ | 0.1725 | 0.345 |
| | KH₂PO₄ | 0.03 | 0.06 |
| | deionized H₂O | | |
| formalin | | 0.412 (0.04% | 0.824 |
| | | formalin) | |
| Helicobacter cells | | 3.6 | 7.3 |

LR2 Adjuvant

| Ingredient | Origin |
|---|---|
| Ethoxylated oleic alcohol 2OE (Oleth-2) | vegetable-derived |
| Macrogol oleic ther 5OE (Oleth-5) | vegetable-derived |
| Pluronic F127 (Poloxamer 407) | synthetic |
| Parafin oil | mineral-derived |
| Physiological Phosphate buffer | mineral-derived |

### Example 38

### Helicobacter Isolates

Two bacterial isolates (2662 and 1268) were recovered from porcine gastric mucosa by micro-aerophilic culture and passageon Skirrow's medium plates.

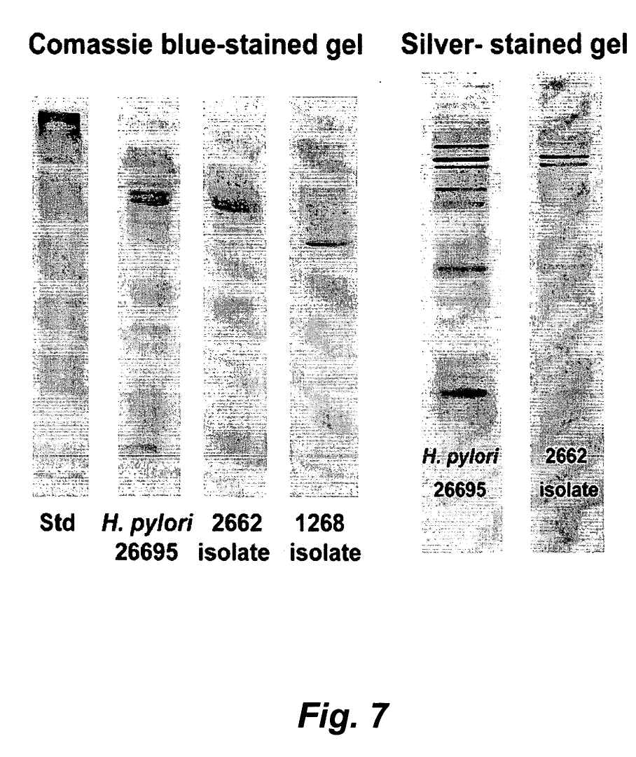

On the basis of gastric location (cardiac and antrum), morphology (Gram-negative, short, curved “gull wing-like” rods), urease activity, reactivity with a rabbit anti-Hp antibody, both isolates were assigned to the genus Helicobacter based on SDS-PAGE and Western blotting profiles, as shown in FIG. 7), isolate 2662 was found to be similar to helicobacter pylori and given the name “Helicobacter cerdo”. Isolate 1268 had a distinctive profile unlike that of 2662.

### Example 39

### Bacterial Load Measured by Urease Activity

TABLE 28
Bacterial Load Measured by Urease Activity

Urease Activity in Gastric Mucosa

| Groups | None | Weak | Moderate | Strong |
|---|---|---|---|---|
| Merial A | 4* | 0 | 2 | — |
| (protease digest) | | | | |
| Merial B | 1 | 4 | 1 | — |
| (formalin whole) | | | | |
| Merial C | 2 | 1 | 3 | — |
| (sonicate, formalin) | | | | |
| Infection Controls | — | 1 | 3 | 3 |

*Gross lesions pars esophagea

### Example 40

### Gastric Inflammatory Response

The gastric inflammatory response (Table 29) was “scored” for follicles and lymphocytic infiltrates into the gastric lamina propria on a scale of 0, none; 1, mild; 2, moderate; and 3, severe. The total inflammatory score for each pig was calculated as the sum of the histologic scores in the gastric cardia and antrum. Group mean scores were calculated from these.

TABLE 29
Gastric Inflammatory Response

| Group Number | Mean Total Score |
|---|---|
| A | 3.6 |
| B | 4.4 |
| C | 4.3 |
| D | 4.4 |
| (Control) | |

### Example 41

### Serologic Responses

TABLE 30
Serologic responses

| Groups | Day 6 | | Day 14 | | Day 24 | Day 40 |
|---|---|---|---|---|---|---|
| Merial A | 1.0 | First | 1.3 | Second | 81.0 | 86.5 |
| (protease | | Vacci- | | Vaccination | | |
| digest) | | nation | | | | |
| Merial B | 1.8 | | 0.0 | | 3.2 | 13.7 |
| (formalin | | | | | | |
| whole) | | | | | | |
| Merial C | 1.0 | | 0.5 | | 22.5 | 25.7 |
| (sonicate, | | | | | | |
| formalin | | | | | | |
| Infection | 1.0 | | 0.8 | | 1.3 | 4.5 |
| Controls | | | | | | |
| Suivaxyn | 6.9 | | 11.8 | | 99.9 | 97.3 |
| Myco | | | | | | |
| hyo + | | | | | | |
| H. cerdo | | | | | | |
| Controls | 9.4 | | 6.6 | | 9.1 | 9.0 |

Vaccine A provided the best indices of protective immunity based upon the intensity of urease activity, blinded histologic evaluation of tissue sections and the strongest serological responses. A combination vaccine of Suivaxyn Myco hyo and H. cerdo and Vaccine A gave smilar antibody responses.

### Example 42

### Animals and Methods for Vaccination with PCVII/H. cerdo-Based Combination Vaccines

The animals used in this study are selected conventional pigs that are PRRSV-free, and M. hyopneumoniae-free.

Conventional piglets will be selected while still with their mothers. All pigs will be weighed and their weights recorded. The pigs will be assigned to 4 groups of at least 5 pigs each, with stratification by weight, sex and litter of origin by Cerberus personnel. All pigs will be examined to ensure health status. Only clinically healthy animals will be included in the trial. All pigs will be identified by a sequentially numbered ear tag in the left ear. A new ear tag with the same identification number will be installed if an animal loses its ear tag.

Pigs will be vaccinated at 1 week and 2 weeks of age while with their mothers. Three groups of pigs will be vaccinated and a group left as unvaccinated control. Vaccinated animals will receive 1 dose (2 ml per dose) via the intramuscular route, as 0.5 ml over each shoulder and hip.

Administered vaccines will be either H cerdo Vaccine A, B or C alone (prepared as described in Examples 25, 26 and 37 above, or H. cerdo Vaccine A, B, or C combined with a PCVII vaccine. Unvaccinated controls will not receive any injection. Vaccination will be by using inactivated PCVII having a titer before inactivation of 10^{6.6} TCID50 formulated in an oil-in-water emulsion. The volume for one dose of vaccine will be 2 ml.

Sixteen piglets (14 days old) will be allocated in two groups of animals, one as a control group the other as a vaccinated group. The animals of the vaccinated group will be injected by intramuscular route at days 0 and 21. Four control group piglets will be injected with a physiological solution. Vaccinated and control animals will then be challenged day 35 by oronasal administration of a PCVII viral suspension, 5 ml in each nostril (5.5. DICC50 per nostril).

Antibodies production: Antibodies in sera may be measured by immunofluorescence as shown in the following table.

At 3 weeks of age, all pigs will be weaned and challenged. Each group will be housed in separate pens in an isolation facility. The pigs will be necropsied at approximately 28 days post challenge. The negative control pigs will be necropsied at the final necropsy date. The experimental design is summarized in Table 31.

TABLE 31
Experimental Design

| Treatment Description | Helicobacter challenge | No challenge |
|---|---|---|
| Vaccine A | 5 pigs | |
| Vaccine B | 5 pigs | |
| Vaccine C | 5 pigs | |
| Negative Controls | | 5 pigs |
| Vaccine A + PCVII | 5 pigs | |
| Vaccine B + PCVII | 5 pigs | |
| Vaccine C + PCVII | 5 pigs | |
| +PCVII | 5 pigs | |

Challenge Procedure and Evaluation: In case of severe clinical illness, treatments that are considered necessary for the animal's welfare may be administered. Each animal's ear tag number, date(s) of illness, presumptive diagnosis, treatment regimen, and disposition of the animal will be recorded. No treatment will be provided following challenge. A moribund or injured animal will be euthanized. An unhealthy animal (clinical illness or injury) may be withdrawn from the study.

Serology and Skin tests: Blood will be collected from the anterior vena cava prior to vaccination, prior to challenge and at necropsy. Helicobacter antibody levels will be determined using a fluorescence-based technique. Antibodies against other pathogens might be assayed as needed. Skin tests will be performed.

Production Parameters: Pigs will be weighed upon arrival, prior to vaccination, prior to challenge and at each necropsy to evaluate potential weight gain or loss.

Necropsy: Pigs will be necropsied at 28 DPI as adapted to Helicobacter challenge.

Having thus described in detail preferred embodiments of the present invention, it is to be understood that the invention defined by the appended claims is not to be limited to particular details set forth in the above description, as many apparent variations thereof are possible without departing from the spirit or scope of the present invention. Modifications and variations of the method and apparatuses described herein will be obvious to those skilled in the art, and are intended to be encompassed by the following claims.

We claim:

1. An immunogenic composition for eliciting an immunological response against a Helicobacter species and porcine circovirus comprising at least one Helicobacter antigen and at least one porcine circovirus antigen, and a veterinarily acceptable vehicle or excipient.

2. The immunogenic composition of claim 1 wherein the porcine circovirus antigen comprises at least one porcine circovirus type II antigen.

3. The immunogical composition of claim 1, wherein the Helicobacter antigen is an antigen of Helicobacter cerdo, Helicobacter heilmanii, or Helicobacter pylori.

4. The immunogenic composition of claim 2 wherein the porcine circovirus type II antigen is at least one antigen of a porcine circovirus type II deposited at the ECACC selected from group consisting of: porcine circovirus type II accession No. V97100219, porcine circovirus type II accession No. V97100218, porcine circovirus type II accession No. V97100217, porcine circovirus type II accession No. V98011608, and porcine circovirus type II accession No. V98011609.

5. The immunogenic composition of claim 2 wherein the porcine circovirus type II antigen is an attenuated virus porcine circovirus type II or an inactivated porcine circovirus type II.

6. The immunogenic composition of claim 1 further comprising a veterinarily acceptable adjuvant and, optionally, a freeze-drying stabilizer.

7. The immunogical composition of claim 3, wherein the Helicobacter antigen is an antigen of Helicobacter cerdo.

8. The immunogenic composition of claim 2 wherein the porcine circovirus type II antigen comprises an antigen encoded by a porcine circovirus type II open reading frame (ORF) selected from the group consisting of the PCVII strain 1010 ORFs 1, 2, 3, 4, 5, 6, 7, 8, 9, 10, 11, 12, and 13 or the equivalent thereof.

9. The immunogenic composition of claim 2 wherein the porcine circovirus type II antigen comprises a vector that contains and expresses in vivo an antigen encoded by a porcine circovirus type II open reading frame (ORF) selected from the group consisting of the PCVII strain 1010 ORFs 1, 2, 3, 4, 5, 6, 7, 8, 9, 10, 11, 12, and 13 or the equivalent thereof.

10. The immunogenic composition of claim 9 wherein the vector is selected from the group consisting of a DNA plasmid, a linear DNA molecule, and a recombinant virus.

11. The immunogenic composition of claim 10 wherein the recombinant virus is selected from the group consisting of pig herpes virus, porcine adenovirus, and poxvirus.

12. The immunogenic composition of claim 11 wherein the recombinant virus is selected from the group consisting of Aujesky's disease virus, vaccinia virus, avipox virus, canarypox virus, and swine pox virus.

13. The immunogenic composition of claim 1 wherein the Helicobacter antigen is selected from the group consisting of an attenuated Helicobacter strain, an inactivated Helicobacter strain, a subunit of a Helicobacter strain, and wherein the porcine circovirus antigen is selected from the group consisting of an attenuated porcine circovirus, an inactivated porcine circovirus, a subunit of porcine circovirus, and a vector that contains and expresses in vivo a nucleic acid molecule encoding the porcine circovirus antigen and is selected from the group consisting of a DNA plasmid, a linear DNA molecule, and a recombinant virus; and optionally an additional antigen of another porcine pathogen.

14. The immunogenic composition of claim 1, further comprising an additional antigen of another porcine pathogen.

15. The immunogenic composition of claim 14 wherein the additional antigen of another porcine pathogen is selected from the group consisting of: an antigen of PRRS virus, an antigen of Mycoplasma hypopneumoniae, an antigen of Actinobacillus pleuropneumoniae, an antigen of E. coli, an antigen of Atrophic Rhinitis, an antigen of Pseudorabies virus, an antigen of Hog cholera, an antigen of Swine Influenza, and combinations thereof.

16. The immunogenic composition according to claim 1 wherein the antigen of porcine circovirus comprises antigens of a plurality of porcine circoviruses.

17. A method for inducing an immunological response against Helicobacter strain and porcine circovirus comprising administering to a porcine an immunogenic composition as claimed in any one of claims 1-16.

18. A kit for preparing the immunogenic composition of claim 1 comprising (i) the at least one Helicobacter antigen and (ii) the at least one porcine circovirus antigen, wherein (i) and (ii) are packaged separately.

19. The kit of claim 18 wherein the porcine circovirus antigen comprises at least one porcine circovirus type II antigen.


SEQUENCE LISTING (32 sequences)

SEQ ID NO: 1 - DNA, 1768 bp, Porcine circovirus Type 2
accagcgcacttcggcagcggcagcacctcggcagcacctcagcagcaacatgcccagcaagaagaatggaagaagcggaccccaaccacataaaaggtgggtgttcacgctgaataatccttccgaagacgagcgcaagaaaatacgggagctcccaatctccctatttgattattttattgttggcgaggagggtaatgaggaaggacgaacacctcacctccaggggttcgctaattttgtgaagaagcaaacttttaataaagtgaagtggtatttgggtgcccgctgccacatcgagaaagccaaaggaactgatcagcagaataaagaatattgcagtaaagaaggcaacttacttattgaatgtggagctcctcgatctcaaggacaacggagtgacctgtctactgctgtgagtaccttgttggagagcgggattctggtgaccgttgcaaagcagcaccctgtaacgtttgtcaaaaatttccgcgggctggctgaacttttgaaagtgagcgggaaaatgcaaaagcgtgattggaaaaccaatgtacacttcattgtggggccacctgggtgtggtaaaagcaaatgggctgctaattttgcaaacccggaaaccacatactggaaaccacctaaaaacaagtggtgggatggttaccatggtgaaaaagtggttgttattgatgacttttatggctggctgccgtgggatgatctactgagactgtgtgatcgatatccattgactgtaaaaactaaaggtggaactgtaccttttttggcccgcagtattctgattaccagcaatcagaccccgttggaatggtactcctcaactgctgtcccagctgtagaagctctctatcggaggattacttccttggtattttggaagaatgctacaaaacaatccacggaggaagggggccagttcgtcaccctttcccccccatgccctgaatttccatatgaaataaattactgagtcttttttatcacttcgtaatggtttttattattcatttagggttcaagtggggggtctttaagattaaattctctgaattgtacatacatggttacacggatattgtagtcctggtcgtatttactgttttcgaacgcagtgccgaggcctacgtggtccacatttccagaggtttgtagcctcagccaaagctgattccttttgttatttggttggaagtaatcaatagtggagtcaagaacaggtttgggtgtgaagtaacgggagtggtaggagaagggttgggggattgtatggcgggaggagtagtttacatatgggtcataggttagggctgtggcctttgttacaaagttatcatctaaaataacagcagtggagcccactcccctatcaccctgggtgatgggggagcaaggccagaattcaaccttaacctttcttattctgtagtattcaaagggtatagagattttgttggtcccccctcccgggggaacaaagtcgtcaattttaaatctcatcatgtccaccgcccaggagggcgttgtgactgtggtacgcttgacagtatatccgaaggtgcgggagaggcgggtgttgaagatgccatttttccttctccaacggtagcggtggcgggggtggacgagccaggggcggcggcggaggatctggccaagatggctgcgggggcggtgtcttcttctgcggtaacgcctccttggatacgtcatagctgaaaacgaaagaagtgcgctgtaagtatt

SEQ ID NO: 2 - DNA, 1759 bp, Porcine circovirus Type 1
accagcgcacttcggcagcggcagcacctcggcagcgtcagtgaaaatgccaagcaagaaaagcggcccgcaaccccataagaggtgggtgttcacccttaataatccttccgaggaggagaaaaacaaaatacgggagcttccaatctccctttttgattattttgtttgcggagaggaaggtttggaagagggtagaactcctcacctccaggggtttgcgaattttgctaagaagcagacttttaacaaggtgaagtggtattttggtgcccgctgccacatcgagaaagcgaaaggaaccgaccagcagaataaagaatactgcagtaaagaaggccacatacttatcgagtgtggagctccgcggaaccaggggaagcgcagcgacctgtctactgctgtgagtacccttttggagacggggtctttggtgactgtagccgagcagttccctgtaacgtatgtgagaaatttccgcgggctggctgaacttttgaaagtgagcgggaagatgcagcagcgtgattggaagacagctgtacacgtcatagtgggcccgcccggttgtgggaagagccagtgggcccgtaattttgctgagcctagggacacctactggaagcctagtagaaataagtggtgggatggatatcatggagaagaagttgttgttttggatgatttttatggctggttaccttgggatgatctactgagactgtgtgaccggtatccattgactgtagagactaaagggggtactgttccttttttggcccgcagtattttgattaccagcaatcaggccccccaggaatggtactcctcaactgctgtcccagctgtagaagctctctatcggaggattactactttgcaattttggaagactgctggagaacaatccacggaggtacccgaaggccgatttgaagcagtggacccaccctgtgcccttttcccatataaaataaattactgagtcttttttgttatcacatcgtaatggtttttatttttatttatttagagggtcttttaggataaattctctgaattgtacataaatagtcagccttaccacataattttgggctgtggctgcattttggagcgcatagccgaggcctgtgtgctcgacattggtgtgggtatttaaatggagccacagctggtttcttttattatttgggtggaaccaatcaattgtttggtccagctcaggtttgggggtgaagtacctggagtggtaggtaaagggctgccttatggtgtggcgggaggagtagttaatataggggtcataggccaagttggtggagggggttacaaagttggcatccaagataacaacagtggacccaacacctctttgattagaggtgatggggtctctggggtaaaattcatatttagcctttctaatacggtagtattggaaaggtaggggtagggggttggtgccgcctgagggggggaggaactggccgatgttgaatttgaggtagttaacattccaagatggctgcgagtatcctccttttatggtgagtacaaattctgtagaaaggcgggaattgaagatacccgtctttcggcgccatctgtaacggtttctgaaggcggggtgtgccaaatatggtcttctccggaggatgtttccaagatggctgcgggggcgggtccttcttctgcggtaacgcctccttggccacgtcatcctataaaagtgaaagaagtgcgctgctgtagtatt

SEQ ID NO: 3 - protein, 314 aa, Porcine circovirus Type 2
MPSKKNGRSGPQPHKRWVFTLNNPSEDERKKIRELPISLFDYFIVGEEGNEEGRTPHLQGFANFVKKQTFNKVKWYLGARCHIEKAKGTDQQNKEYCSKEGNLLIECGAPRSQGQRSDLSTAVSTLLESGILVTVAEQHPVTFVKNFRGLAELLKVSGKMQKRDWKTNVHFIVGPPGCGKSKWAANFANPETTYWKPPKNKWWDGYHGEKVVVIDDFYGWLPWDDLLRLCDRYPLTVKTKGGTVPFLARSILITSNQTPLEWYSSTAVPAVEALYRRITSLVFWKNATKQSTEEGGQFVTLSPPCPEFPYEINY

SEQ ID NO: 4 - protein, 312 aa, Porcine circovirus Type 1
MPSKKSGPQPHKRWVFTLNNPSEEEKNKIRELPISLFDYFVCGEEGLEEGRTPHLQGFANFAKKQTFNKVKWYFGARCHIEKAKGTDQQNKEYCSKEGHILIECGAPRNQGKRSDLSTAVSTLLETGSLVTVAEQFPVTYVRNFRGLAELLKVSGKMQQRDWKTAVHVIVGPPGCGKSQWARNFAEPRDTYWKPSRNKWWDGYHGEEVVVLDDFYGWLPWDDLLRLCDRYPLTVETKGGTVPFLARSILITSNQAPQEWYSSTAVPAVEALYRRITTLQFWKTAGEQSTEVPEGRFEAVDPPCALFPYKINY

SEQ ID NO: 5 - protein, 261 aa, Porcine circovirus Type 2
MLLLRCCRGAAAAEVRWYYSSALLSFSAMTYPRRRYRRRRHRPRSHLGQILRRRPWLVHPRHRYRWRRKNGIFNTRLSRTFGYTVKRTTVTTPSWAVDMMRFKIDDFVPPGGGTNKISIPFEYYRIRKVKVEFWPCSPITQGDRGVGSTAVILDDNFVTKATALTYDPYVNYSSRHTIPQPFSYHSRYFTPKPVLDSTIDYFQPNNKRNQLWLRLQTSGNVDHVGLGTAFENSKYDQDYNIRVTMYVQFREFNLKDPPLEP

SEQ ID NO: 6 - protein, 231 aa, Porcine circovirus Type 1
WPRRRYRRRRTRPRSHLGNILRRRPYLAHPAFRNRYRWRRKTGIFNSRLSTEFVLTIKGGYSQPSWNVNYLKFNIGQFLPPSGGTNPLPLPFQYYRIRKAKYEFYPRDPITSNQRGVGSTVVILDANFVTPSTNLAYDPYINYSSRHTIRQPFTYHSRYFTPKPELDQTIDWFHPNNKRNQLWLHLNTHTNVEHTGLGYALQNAATAQNYVVRLTIYVQFREFILKDPLNK

SEQ ID NO: 7 - protein, 59 aa, Porcine circovirus Type 2
MTCTLVFQSRFCIFPLTFKSSASPRKFLTNVTGCCSATVTRLPLSNKVLTAVDRSLRCP

SEQ ID NO: 8 - protein, 115 aa, Porcine circovirus Type 1
MTCTAVFQSRCCIFPLTFKSSASPRKFLTYVTGNCSATVTKDPVSKRVLTAVDRSLRFPWFRGAPHSISMWPSLLQYSLFCWSVPFAFSMWQRAPKYHFTLLKVCFLAKFANPWR

SEQ ID NO: 9 - protein, 104 aa, Porcine circovirus Type 2
MVTIPPLVSRWFPVCGFRVCKISSPFAFTTPRWPHNDVYIGLPITLLHFPAHFQKFSQPAEISDKRYRVLLCNGHQTPALQQGTHSSRQVTPLSLRSRSSTFNK

SEQ ID NO: 10 - protein, 206 aa, Porcine circovirus Type 1
MISIPPLISTRLPVGVPRLSKITGPLALPTTGRAHYDVYSCLPITLLHLPAHFQKFSQPAEISHIRYRELLGYSHQRPRLQKGTHSSRQVAALPLVPRSSTLDKYVAFFTAVFFILLVGSFRFLDVAAGTKIPLHLVKSLLLSKIRKPLEVRSSTLFQTFLSANKIIKKGDWKLPYFVFLLLGRIIKGEHPPLMGLRAAFLAWHFH

SEQ ID NO: 11 - DNA, 1768 bp, Porcine circovirus Type 2
accagcgcacttcggcagcggcagcacctcggcagcacctcagcagcaacatgcccagcaagaagaatggaagaagcggaccccaaccacataaaaggtgggtgttcacgctgaataatccttccgaagacaagcgcaagaaaatacgggagctcccaatctccctatttgattattttattgttggcgaggagggtaatgaggaaggacgaacacctcacctccaggggttcgctaattttgtgaagaagcaaacttttaataaagtgaagtggtatttgggtgcccgctgccacatcgagaaagccaaaggaactgatcagcagaataaagaatattgcagtaaagaaggcaacttacttattgaatgtggagctcctcgatctcaaggacaacggagtgacctgtctactgctgtgagtaccttgttggagagcgggattctggtgaccgttgcaaagcagcaccctgtaacgtttgtcaaaaatttccgcgggctggctgaacttttgaaagtgagcgggaaaatgcaaaagcgtgattggaaaaccaatgtacacttcattgtggggccacctgggtgtggtaaaagcaaatgggctgctaattttgcaaacccggaaaccacatactggaaaccacctaaaaacaagtggtgggatggttaccatggtgaaaaagtggttgttattgatgacttttatggctggctgccgtgggatgatctactgaaactgtgtgatcgatatccattgactgtaaaaactaaaggtggaactgtaccttttttggcccgcagtattctgattaccagcaatcagaccccgttggaatggtactcctcaactgctgtcccagctgtagaagctctctatcggaggattacttccttggtattttggaagaatgctacagaacaatccacggaggaagggggccagtttgtcaccctttcccccccatgccctgaatttccatatgaaataaattactgagtcttttttatcacttcgtaatggtttttattattcatttagggtttaagtggggggtctttaagattaaattctctgaattgtacatacatggttacacggatattgtagtcctggtcgtatttactgttttcgaacgcagtgccgaggcctacgtggtccacatttccagaggtttgtagcctcagccaaagctgattccttttgttatttggttggaagtaatcaatagtggagtcaagaacaggtttgggtgtgaagtaacgggagtggtaggagaagggttgggggattgtatggcgggaggagtagtttacatatgggtcataggttagggctgtggcctttgttacaaagttatcatctaaaataacagcagtggagcccactcccctatcaccctgggtgatgggggagcagggccagaattcaaccttaacctttcttattctgtagtattcaaagggtatagagattttgttggtcccccctcccgggggaacaaagtcgtcaattttaaatctcatcatgtccaccgcccaggagggcgttgtgactgtggtacgcttgacagtatatccgaaggtgcgggagaggcgggtgttgaagatgccatttttccttctccaacggtagcggtggcgggggtggacgagccaggggcggcggcggaggatctggccaagatggctgcgggggcggtgtcttcttctgcggtaacgcctccttggatacgtcatagctgaaaacgaaagaagtgcgctgtaagtatt

SEQ ID NO: 12 - DNA, 1640 bp, Porcine circovirus Type 2
accagcgcacttcggcagcggcagcacctcggcagcacctcagcagcaacatgcccagcaagaagaatggaagaagcggaccccaaccacataaaaggtgggtgttcacgctgaataatccttccgaagacaagcgcaagaaaatacgggagctcccaatctccctatttgattattttattgttggcgaggagggtaatgaggaaggacgaacacctcacctccaggggttcgctaatttgtggagctcctcgatctcaaggacaacggagtgacctgtctactgctgtgagtaccttgttggagagcgggattctggtgaccgttgcaaagcagcaccctgtaacgtttgtcaaaaatttccgcgggctggctgaacttttgaaagtgagcgggaaaatgcaaaagcgtgattggaaaaccaatgtacacttcattgtggggccacctgggtgtggtaaaagcaaatgggctgctaattttgcaaacccggaaaccacatactggaaaccacctaaaaacaagtggtgggatggttaccatggtgaaaaagtggttgttattgatgacttttatggctggctgccgtgggatgatctactgaaactgtgtgatcgatatccattgactgtaaaaactaaaggtggaactgtaccttttttggcccgcagtattctgattaccagcaatcaaaccccgttggaatggtactcctcaactgctgtcccagctgtagaagctctctatcggaggattacttccttggtattttggaagaatgttacagaacaatccacggaggaagggggccagtttgtcaccctttcccccccatgccctgaatttccatatgaaataaattactgagtcttttttatcacttcgtaatggtttttattattcatttagggtttaagtggggggtctttaagattaaattctctgaattgtacatacatggttacacggatattgtagtcctggtcgtatttactgttttcgaacgcagtgccgaggcctacgtggtccacatttctagaggtttgtagcctcagccaaagctgattccttttgttatttggttggaagtaatcaatagtggagtcaagaacaggtttgggtgtgaagtaacgggagtggtaggagaagggttgggggattgtatggcgggaggagtagtttacatatgggtcataggttagggctgtggcctttgttacaaagttatcatctagaataacagcagtggagcccactcccctatcaccctgggtgatgggggagcagggccagaattcaaccttaacctttcttattctgtagtattcaaagggtatagagattttgttggtcccccctcccgggggaacaaagtcgtcaattttaaatctcatcatgtccaccgcccaggagggcgttgtgactgtggtaggcttgacagtatatccgaaggtgcgggagaggcgggtgttgaagatgccatttttccttctccaacggtagcggtggcgggggtggacgagccaggggcggcggcggaggatctggccaagatggctgcgggggcggtgtcttcttctgcggtaacgcctccttggatacgtcatagctgaaaacgaaagaagtgcgctgtaagtatt

SEQ ID NO: 13 - DNA, 18 bp, Porcine circovirus Type 2
actacagcagcgcacttc

SEQ ID NO: 14 - DNA, 30 bp, Porcine circovirus Type 2
aaaaaagactcagtaatttatttcatatgg

SEQ ID NO: 15 - DNA, 23 bp, Porcine circovirus Type 2
atcacttcgtaatggtttttatt

SEQ ID NO: 16 - DNA, 18 bp, Porcine circovirus Type 2
tgcggtaacgcctccttg

SEQ ID NO: 17 - DNA, 21 bp, Porcine circovirus Type 2
ctacagctgggacagcagttg

SEQ ID NO: 18 - DNA, 23 bp, Porcine circovirus Type 2
catacatggttacacggatattg

SEQ ID NO: 19 - DNA, 20 bp, Porcine circovirus Type 2
ccgcaccttcggatatactg

SEQ ID NO: 20 - protein, 59 aa, Porcine circovirus Type 2
MYTSLWGHLGVVKANGLLILQTRKPHTGNHLKTSGGMVTMVKKWLLLMTFMAGCRGMIY

SEQ ID NO: 21 - protein, 53 aa, Porcine circovirus Type 2
MVFIIHLGFKWGVFKIKFSELYIHGYTDIVVLVVFTVFERSAEAYVVHISRGL

SEQ ID NO: 22 - DNA, 20 bp, Porcine circovirus Type 2
tcccgttacttcacacccaa

SEQ ID NO: 23 - DNA, 20 bp, Porcine circovirus Type 2
cctgtctactgctgtgagta

SEQ ID NO: 24 - DNA, 1768 bp, Porcine circovirus Type 2
aattcaaccttaacctttcttattctgtagtattcaaagggtatagagattttgttggtcccccctcccgggggaacaaagtcgtcaattttaaatctcatcatgtccaccgcccaggagggcgttgtgactgtggtacgcttgacagtatatccgaaggtgcgggagaggcgggtgttgaagatgccatttttccttctccaacggtagcggtggcgggggtggacgagccaggggcggcggcggaggatctggccaagatggctgcgggggcggtgtcttcttctgcggtaacgcctccttggatacgtcatagctgaaaacgaaagaagtgcgctgtaagtattaccagcgcacttcggcagcggcagcacctcggcagcacctcagcagcaacatgcccagcaagaagaatggaagaagcggaccccaaccacataaaaggtgggtgttcacgctgaataatccttccgaagacgagcgcaagaaaatacgggagctcccaatctccctatttgattattttattgttggcgaggagggtaatgaggaaggacgaacacctcacctccaggggttcgctaattttgtgaagaagcaaacttttaataaagtgaagtggtatttgggtgcccgctgccacatcgagaaagccaaaggaactgatcagcagaataaagaatattgcagtaaagaaggcaacttacttattgaatgtggagctcctcgatctcaaggacaacggagtgacctgtctactgctgtgagtaccttgttggagagcgggagtctggtgaccgttgcagagcagcaccctgtaacgtttgtcagaaatttccgcgggctggctgaacttttgaaagtgagcgggaaaatgcagaagcgtgattggaagaccaatgtacacgtcattgtggggccacctgggtgtggtaaaagcaaatgggctgctaattttgcagacccggaaaccacatactggaaaccacctagaaacaagtggtgggatggttaccatggtgaagaagtggttgttattgatgacttttatggctggctgccgtgggatgatctactgagactgtgtgatcgatatccattgactgtagagactaaaggtggaactgtaccttttttggcccgcagtattctgattaccagcaatcagaccccgttggaatggtactcctcaactgctgtcccagctgtagaagctctctatcggaggattacttccttggtattttggaagaatgctacagaacaatccacggaggaagggggccagttcgtcaccctttcccccccatgccctgaatttccatatgaaataaattactgagtcttttttatcacttcgtaatggtttttattattcatttagggtttaagtggggggtctttaagattaaattctctgaattgtacatacatggttacacggatattgtagtcctggtcgtatttactgttttcgaacgcagcgccgaggcctacgtggtccacatttccagaggtttgtagtctcagccaaagctgattccttttgttatttggttggaagtaatcaatagtggagtcaagaacaggtttgggtgtgaagtaacgggagtggtaggagaagggttgggggattgtatggcgggaggagtagtttacatatgggtcataggttagggctgtggcctttgttacaaagttatcatctagaataacagcagtggagcccactcccctatcaccctgggtgatgggggagcagggccag

SEQ ID NO: 25 - DNA, 1767 bp, Porcine circovirus Type 2
aattcaaccttaacctttcttattctgtagtattcaaagggcacagagcgggggtttgagccccctcctgggggaagaaagtcattaatattgaatctcatcatgtccaccgcccaggagggcgttctgactgtggttcgcttgacagtatatccgaaggtgcgggagaggcgggtgttgaagatgccatttttccttctccagcggtaacggtggcgggggtggacgagccaggggcggcggcggaggatctggccaagatggctgcgggggcggtgtcttcttctccggtaacgcctccttggatacgtcatatctgaaaacgaaagaagtgcgctgtaagtattaccagcgcacttcggcagcggcagcacctcggcagcacctcagcagcaacatgccgagcaagaagaatggaagaagcggaccccaaccccataaaaggtgggtgttcactctgaataatccttccgaagacgagcgcaagaaaatacgggatcttccaatatccctatttgattattttattgttggcgaggagggtaatgaggaaggacgaacacctcacctccaggggttcgctaattttgtgaagaagcagacttttaataaagtgaagtggtatttgggtgcccgctgccacatcgagaaagcgaaaggaacagatcagcagaataaagaatactgcagtaaagaaggcaacttactgatggagtgtggagctcctagatctcagggacaacggagtgacctgtctactgctgtgagtaccttgttggagagcgggagtctggtgaccgttgcagagcagcaccctgtaacgtttgtcagaaatttccgcgggctggctgaacttttgaaagtgagcgggaaaatgcagaagcgtgattggaagactaatgtacacgtcattgtggggccacctgggtgtggtaaaagcaaatgggctgctaattttgcagacccggaaaccacatactggaaaccacctagaaacaagtggtgggatggttaccatggtgaagaagtggttgttattgatgacttttatggctggctgccctgggatgatctactgagactgtgtgatcgatatccattgactgtagagactaaaggtggaactgtaccttttttggcccgcagtattctgattaccagcaatcagaccccgttggaatggtactcctcaactgctgtcccagctgtagaagctctttatcggaggattacttccttggtattttggaagaatgctacagaacaatccacggaggaagggggccagttcgtcaccctttcccccccatgccctgaatttccatatgaaataaattactgagtcttttttatcacttcgtaatggtttttattattcattaagggttaagtggggggtctttaagattaaattctctgaattgtacatacatggttacacggatattgtattcctggtcgtatatactgttttcgaacgcagtgccgaggcctacgtggtctacatttccagcagtttgtagtctcagccacagctggtttcttttgttgtttggttggaagtaatcaatagtggaatctaggacaggtttgggggtaaagtagcgggagtggtaggagaagggctgggttatggtatggcgggaggagtagtttacataggggtcataggtgagggctgtggcctttgttacaaagttatcatctagaataacagcactggagcccactcccctgtcaccctgggtgatcggggagcagggccag

SEQ ID NO: 26 - DNA, 1767 bp, Porcine circovirus Type 2
aattcaaccttaacctttcttattctgtagtattcaaagggcacagagcgggggtttgagccccctcctgggggaagaaagtcattaatattgaatctcatcatgtccaccgcccaggagggcgttttgactgtggttcgcttgacagtatatccgaaggtgcgggagaggcgggtgttgaagatgccatttttccttctccagcggtaacggtggcgggggtggacgagccaggggcggcggcggaggatctggccaagatggctgcgggggcggtgtcttcttctccggtaacgcctccttggatacgtcatatctgaaaacgaaagaagtgcgctgtaagtattaccagcgcacttcggcagcggcagcacctcggcagcacctcagcagcaacatgcccagcaagaagaatggaagaagcggaccccaaccccataaaaggtgggtgttcactctgaataatccttccgaagacgagcgcaagaaaatacgggatcttccaatatccctatttgattattttattgttggcgaggagggtaatgaggaaggacgaacacctcacctccaggggttcgctaattttgtgaagaagcagacttttaataaagtgaagtggtatttgggtgcccgctgccacatcgagaaagcgaaaggaacagatcagcagaataaagaatactgcagtaaagaaggcaacttactgatggagtgtggagctcctagatctcagggacaacggagtgacctgtctactgctgtgagtaccttgttggagagcgggagtctggtgaccgttgcagagcagcaccctgtaacgtttgtcagaaatttccgcgggctggctgaacttttgaaagtgagcgggaaaatgcagaagcgtgattggaagactaatgtacacgtcattgtggggccacctgggtgtggtaaaagcaaatgggctgctaattttgcagacccggaaaccacatactggaaaccacctagaaacaagtggtgggatggttaccatggtgaagaagtggttgttattgatgacttttatggctggctgccctgggatgatctactgagactgtgtgatcgatatccattgactgtagagactaaaggtggaactgtaccttttttggcccgcagtattctgattaccagcaatcagaccccgttggaatggtactcctcaactgctgtcccagctgtagaagctctttatcggaggattacttccttggtattttggaagaatgctacagaacaatccacggaggaagggggccagttcgtcaccctttcccccccatgccctgaatttccatatgaaataaattactgagtcttttttatcacttcgtaatggtttttattattcattaagggttaagtggggggtctttaagattaaattctctgaattgtacatacatggttacacggatattgtattcctggtcgtatatactgttttcgaacgcagtgccgaggcctacgtggtctacatttccagtagtttgtagtctcagccacagctgatttcttttgttgtttggttggaagtaatcaatagtggaatctaggacaggtttgggggtaaagtagcgggagtggtaggagaagggctgggttatggtatggcgggaggagtagtttacataggggtcataggtgagggctgtggcctttgttacaaagttatcatctagaataacagcactggagcccactcccctgtcaccctgggtgatcggggagcagggccag

SEQ ID NO: 27 - DNA, 1768 bp, Porcine circovirus Type 2
aattcaaccttaaccttttttattctgtagtattcaaagggtatagagattttgttggtcccccctcccgggggaacaaagtcgtcaatattaaatctcatcatgtccaccgcccaggagggcgttctgactgtggtagccttgacagtatatccgaaggtgcgggagaggcgggtgttgaagatgccatttttccttctccaacggtagcggtggcgggggtggacgagccaggggcggcggcggaggatctggccaagatggctgcgggggcggtgtcttcttctgcggtaacgcctccttggatacgtcatagctgaaaacgaaagaagtgcgctgtaagtattaccagcgcacttcggcagcggcagcacctcggcagcacctcagcagcaacatgcccagcaagaagaatggaagaagcggaccccaaccacataaaaggtgggtgttcacgctgaataatccttccgaagacgagcgcaagaaaatacgggagctcccaatctccctatttgattattttattgttggcgaggagggtaatgaggaaggacgaacacctcacctccaggggttcgctaattttgtgaagaagcaaacttttaataaagtgaagtggtatttgggtgcccgctgccacatcgagaaagccaaaggaactgatcagcagaataaagaatattgcagtaaagaaggcaacttacttattgaatgtggagctcctcgatctcaaggacaacggagtgacctgtctactgctgtgagtaccttgttggagagcgggagtctggtgaccgttgcagagcagcaccctgtaacgtttgtcagaaatttccgcgggctggctgaacttttgaaagtgagcgggaaaatgcagaagcgtgattggaagaccaatgtacacgtcattgtggggccacctgggtgtggtaaaagcaaatgggctgctaattttgcagacccggaaaccacatactggaaaccacctagaaacaagtggtgggatggttaccatggtgaagaagtggttgttattgatgacttttatggctggctgccgtgggatgatctactgagactgtgtgatcgatatccattgactgtagagactaaaggtggaactgtaccttttttggcccgcagtattctgattaccagcaatcagaccccgttggaatggtactcctcaactgctgtcccagctgtagaagctctctatcggaggattacttccttggtattttggaagaatgctacagaacaatccacggaggaagggggccagttcgtcaccctttcccccccatgccctgaatttccatatgaaataaattactgagtcttttttatcacttcgtaatggtttttattattcatttagggtttaagtggggggtctttaagattaaattctctgaattgtacatacatggttacacggatattgtagtcctggtcgtatatactgttttcgaacgcagtgccgaggcctacgtggtccacatttctagaggtttgtagcctcagccaaagctgattccttttgttatttggttggaagtaatcaatagtggagtcaagaacaggtttgggtgtgaagtaacgggagtggtaggagaagggttgggggattgtatggcgggaggagtagtttacatatgggtcataggttagggctgtggcctttgttacaaagttatcatctagaataacagcagtggagcccactcccctatcaccctgggtgatgggggagcagggccag

SEQ ID NO: 28 - DNA, 1768 bp, Porcine circovirus Type 2
  modified_base (231): a, c, g, t, unknown or other
gaattcaaccttaaccttttttattctgtagtattcaaagggtataaagattttgttggtcccccctcccgggggaacaaagtcgtcaatattaaatctcatcatgtccaccgcccaggagggcgttctgactgtggtagccttgacagtatatccgaaggtgcgggagargcgggtgttgaaaatgccatttttccttctccaacggtagcggtggcgggggtggacmanccacgggcggcggcggawgatctggccaagatggctgcgggggcggtgtcttcttctgcggtaacgcctccttggatacgtcatagctgaaaacgaaagaagtgcgctgtaagtattaccagcgcacttcggcagcggcagcacctcggcagcacctcagcagcaacatgcccagcaagaagaatggaagaagcggaccccaaccacataaaaggtgggtgttcacgctgaataatccttccgaagacgagcgcaagaaaatacgggagctcccaatctccctatttgattattttattgttggcgaggagggtwwtgaggaangacgaacacctcacctccaggggttcgctaattttgtgaagaagcaaacttttaataaagtgaagtggtatttgggtgcccgctgccacatcgagaaagccaaaggaactgatcagcagaataaagaatattgcagtaaagaaggcaacttacttattgaatgtggagctcctcgatctcaaggacaacggagtgacctgtctactgctgtgagtaccttgttggagagcgggagtctggtgaccgttgcagagcagcaccctgtaacgtttgtcagaaatttccgcgggctggctgaacttttgaaagtgagcgggaaaatgcagaagcgtgattggaagaccaatgtacacgtcattgtggggccacctgggtgtggtaaaagcaaatgggctgctaattttgcagacccggaaaccacatactggaaaccacctagaaacaagtggtgggatggttaccatggtgaagaagtggttgttattgatgacttttatggctggctgccgtgggatgatctactgagactgtgtgatcgatatccattgactgtagagactaaaggtggaactgtacnnnnnnnggcccgcagtattctgattaccagcaatcagaccccgttggaatggtactcctcaactgctgtcccagctgtagaagctctctatcggaggattacttccttggtattttggaagaatgctacagaacaatccacggaggaagggggccagttngtcaccctttcccccccatgccctgaatttccatatgaaataaattactgagtcttttttatcacttcgtaatggtttttattattcatttagggtttaagtggggggtctttaagattaaattctctgaattgtacatacatggttacacggatattgtagtcctggtcgtatatactgttttcgaacgcagtgccgaggcctacgtggtccacatttctagaggtttgtagcctcagccaaagctgattccttttgttatttggttggaagtaatcaatagtggagtcaagaacaggtttgggtgtgaagtaacgggagtggtaggagaagggttgggggattgtatggcgggaggagtagtttacatatgggtcataggttagggctgtggcctttgttacaaagttatcatctagaataacagcagtggagcccactcccctatcaccctgggtgatgggggagcagggcca

SEQ ID NO: 29 - DNA, 1768 bp, Porcine circovirus Type 2
accagcgcacttcggcagcggcagcacctcggcagcacctcagcagcaacatgcccagcaagaagaatggaagaagcggaccccaaccacataaaaggtgggtgttcacgctgaataatccttccgaagacgagcgcaagaaaatacgggagctcccaatctccctatttgattattttattgttggcgaggagggtaatgaggaaggacgaacacctcacctccaggggttcgctaattttgtgaagaakcaaacttttaataaagtgaagtggtatttgggtgcccgctgccacatcgagaaagccaaaggaactgatcagcagaataaagaatattgcagtaaagaaggcaacttacttattgaatgtggagctcctcgatctcaaggacaacggagtgacctgtctactgctgtgagtaccttgttggagagcgggagtctggtgaccgttgcagagcagcaccctgtaacgtttgtcagaaatttccgcgggctggctgaacttttgaaagtgagcgggaaaatgcagaagcgtgattggaagaccaatgtacacgtcattgtggggccacctgggtgtggtaaaagcaaatgggctgctaattttgcagacccggaaaccacatactggaaaccacctagaaacaagtggtgggatggttaccatggtgaagaagtggttgttattgatgacttttatggctggctgccgtgggatgatctactgagactgtgtgatcgatatccattgactgtagagactaaaggtggaactgtaccttttttggcccgcagtattctgattaccagcaatcagaccccgttggaatggtactcctcaactgctgtcccagctgtagaagctctctatcggaggattacttccttggtattttggaagaatgctacagaacaatccacggaggaagggggccagttcgtcaccctttcccccccatgccctgaatttccatatgaaataaattactgagtcytttttatcacttcgtaatggtttttattattcatttaggggttaagtggggggtctttaagattaaattccctgaattgtacatacagggttacacggatattgtagtcctggtcgtatttactgttttcgaacgcagtgccgaggcctacgtggtccacatttctagaggtttgtagcctcagccaaagctgattccttttgttatttggttggaagtaatcaatagtggagtcaagaacaggtttgggtgtgaagtaacgggagtggtaggagaagggttgggggattgtatggcgggaggagtagtttacatatgggtcatatgtttgggctgtggcctttggtacaaagttatcatctagaataacagcagtggagcccactcccctatcaccctgggtgatgggggagcagggccagaattcaaccttaacctttcttattctgtagtattcaaagggtatagagattttgttggtcccccctcccgggggaacaaagtcgtcaattttaaatctcatcatgtccaccgcccaggagggcgttgtgactgtggtacgcttgacagtatatccgaaggtgcgggagaggcgggtgttgaagatgccatttttccttctccaacggtagcggtggcgggggtggacgagccaggggcggcggcggaggatctggccaagatggctgcgggggcggtgtcttcttctgcggtaacgcctccttggatatgtcatagctgaaaacgaaagaagtgcgctgtaagtatt

SEQ ID NO: 30 - DNA, 1768 bp, Porcine circovirus Type 2
accagcgcacttcggcagcggcagcacctcggcagcacctcagcagcaacatgcccagcaagaagaatggaagaagcggaccccaaccacataaaaggtgggtgttcacgctgaataatccttccgaagacgagcgcaagaaaatacgggagctcccaatctccctatttgattattttattgttggcgaggagggtaatgaggaaggacgaacacctcacctccaggggttcgctaattttgtgaagaagcaaacttttaataaagtgaagtggtatttgggtgcccgctgccacatcgagaaagccaaaggaactgatcagcagaataaagaatattgcagtaaagaaggcaacttacttattgaatgtggagctcctcgatctcaaggacaacggagtgacctgtctactgctgtgagtaccttgttggagagcgggagtctggtgaccgttgcagagcagcaccctgtaacgtttgtcagaaatttccgcgggctggctgaacttttgaaagtgagcgggaaaatgcagaagcgtgattggaagaccaatgtacacgtcattgtggggccacctgggtgtggtaaaagcaaatgggctgctaattttgcagacccggaaaccacatactggaaaccacctagaaacaagtggtgggatggttaccatggtgaagaagtggttgttattgatgacttttatggctggctgccgtgggatgatctactgagactgtgtgatcgatatccattgactgtagagactaaaggtggaactgtaccttttttggcccgcagtattctgattaccagcaatcagaccccgttggaatggtactcctcaactgctgtcccagctgtagaagctctctatcggaggattacttccttggtattttggaagaatgctacagaacaatccacggaggaagggggccagttcgtcaccctttcccccccatgccctgaatttccatatgaaataaattactgagtcttttttatcacttcgtaatggtttttattattcatttaggggttaagtggggggtctttaagattaaattctctgaattgtacatacatggttacacggatattgtagtcctggtcgtatttactgttttcgaacgcagtgccgaggcctacgtggtccacatttctagaggtttgtagcctcagccaaagctgattccttttgttatttggttggaagtaatcaatagtggagtcaagaacaggtttgggtgtgaagtaacgggagtggtaggagaagggttgggggattgtatggcgggaggagtagtttacatatgggtcataggttagggctgtggcctttggtacaaagttatcatctagaataacagcagtggagcccactcccctatcaccctgggtgatgggggagcagggccagaattcaaccttaaccttttttattctgtagtattcaaagggtatagagattttgttggtcccccctcccgggggaacaaagtcgtcaattttaaatctcatcatgtccaccgcccaggagggcgttgtgactgtagtacgcttgacagtatatccgaaggtgcgggagaggcgggtgttgaagatgccatttttccttctccaacggtagcggtggcgggggtggacgagccaggggcggcggcggaggatctggccaagatggctgcgggggcggtgtcttcttctgcggtaacgcctccttggatacgtcatagctgaaaacgaaagaagtgcgctgtaagtatt

SEQ ID NO: 31 - protein, 59 aa, Porcine circovirus Type 2
MKCTLVFQSRFCIFPLTFKSSASPRKFLTNVTGCCFATVTRIPLSNKVLTAVDRSLRCP

SEQ ID NO: 32 - protein, 104 aa, Porcine circovirus Type 2
MVTIPPLVFRWFPVCGFRVCKISSPFAFTTPRWPHNEVYIGFPITLLHFPAHFQKFSQPAEIFDKRYRVLLCNGHQNPALQQGTHSSRQVTPLSLRSRSSTFNK